(19) United States
(12) Patent Application Publication
(10) Pub. No.: US 2024/0238417 A1
(43) Pub. Date: Jul. 18, 2024

(54) METHODS OF PRODUCING TUMOR VACCINES AND USES THEREOF

(71) Applicant: BEYOND AIR, INC., Garden City, NY (US)

(72) Inventors: Amir AVNIEL, Rehovot (IL); Hila CONFINO, Rehovot (IL); Matan GOLDSHTEIN, Rehovot (IL); Steven A. LISI, Garden City, NY (US)

(21) Appl. No.: 18/563,241

(22) PCT Filed: May 25, 2022
(86) PCT No.: PCT/IB2022/054911

§ 371 Date: Nov. 21, 2023

Related U.S. Application Data
(60) Provisional application No. 63/193,163, filed on May 26, 2021.
(60) Provisional application No. 63/193,169, filed on May 26, 2021.
(60) Provisional application No. 63/193,181, filed on May 26, 2021.

(51) Int. Cl. A61K 39/00; A61P 35/00; C12N 5/09

(52) U.S. Cl. CPC A61K 39/461; A61P 35/00; C12N 5/0693; C12N 2500/02; C12N 2500/30

(57) ABSTRACT

Methods of producing tumor vaccine and uses thereof are provided. Accordingly there is provided a method of producing a tumor vaccine, the method comprising ex-vivo exposing a tumor sample to gaseous nitric oxide (gNO); suspending said tumor sample in a medium or buffer subsequent to said exposing, so as obtain tumor cells in suspension; and titrating a pH of said suspension to 6-8. Also provided is provided a method of producing a tumor vaccine, the method comprising ex-vivo exposing a tumor sample to gaseous nitric oxide (gNO); and culturing said tumor sample in a medium comprising antibiotic at a concentration of at least 2 fold higher than the gold standard concentration for culturing primary cells of the same type as said tumor sample. Also provided are vaccines obtainable by the method and uses thereof.

### RELATED APPLICATION/S

This application claims the benefit of priority of U.S. Patent Application No. 63/193,169 filed on May 26, 2021; U.S. Patent Application No. 63/193,163 filed on May 26, 2021; and U.S. Patent Application No. 63/193,181 filed on May 26, 2021, the contents of which are incorporated herein by reference in their entirety.

### FIELD AND BACKGROUND OF THE INVENTION

The present invention, in some embodiments thereof, relates to methods of producing tumor vaccine and uses thereof.

The high mortality rate associated with cancer and its resistance to conventional treatments such as radiation and chemotherapy has led to the investigation of a variety of anti-cancer immunotherapies, such as tumor vaccines. Many types of tumor vaccine have been developed using autologous or allogeneic cancer cells [see e.g. Srivatsan S, et al. Hum Vaccin Immunother. 2014; 10(1):52-63]. Autologous cancer vaccination strategies use material derived from apatient's tumor to create personalized treatments. These treatments vary over a broad spectrum from immunization with patient-specific tumor cell lysates or purified cancer antigens (including those combined with antigen-presenting cells such as dendritic cells), to the induction of anti-tumor immunity with inactivated (e.g. by radiation or fixatives) patient-derived cancer cells. Allogeneic vaccines are largely similar to autologous vaccines with the exception that material is sourced from another member of the same species. Commonly used allogeneic materials include use of established laboratory-grown cancer cell lines known to express tumor associated antigens (TAAs) of a specific tumor type. This allows these therapeutics to be mass-produced, stored, and modified prior to use. Furthermore, cell-based therapies can be modified to increase immunogenicity by transfection with immune stimulatory molecules. For example, the GVAX vaccine platform involves the use of irradiated allogeneic tumor cell lines, modified to secrete the cytokine granulocyte-macrophage colony-stimulating factor (GM-CSF) to augment the immunogenicity of the vaccine.

Nitric oxide (NO) is a short-lived, endogenously produced gas that acts as a signaling molecule in the body (Thomas, D. D. Redox Biol 5, 225-233, 2015). Increasing evidence highlights its wide spectrum of action in different pathologic conditions, including cancer (Huerta, S. Futur. Sci. OA 1, FS044, 2015) and involvement in immune cell signaling against pathogens (Schairer et al. Virulence 3, 271-279, 2012

Preclinical studies testing the effect of exogenously administered nitric oxide (NO) demonstrated its anti-cancer properties and suggested that NO may serve as a potent tumoricidal agent either as a single agent or in combination with other antineoplastic compounds [see e.g. WO 2008/095311; WO 2008/095312; WO 2013/132500; WO 2014/0088490; WO 2013/132503; U.S. Pat. No. 8,168,232; Vannini et al. Redox Biol 6, 334-343, 2015; Seabra, A. B and Durin, N. Eur J Pharmacol 826,158-168, 2018; Ning et al. Biochem Biophys Res Commun 447(3), 537-542, 2014; Huerta, Future Sci. OA (2015) 1(1), FS044; Confino H et al. Gaseous Nitric Oxide at High Concentrations is a Powerful Anti Tumor Agent both in vitro and in vivo. AACR 2020; Confino H et al. Nitric oxide tumor ablation stimulates an anti-tumor immune response in mice, AACR Tumor Immunology and Immunotherapy 2020; and Confino H et al. Nitric Oxide Lung Cancer Active Vaccination, NACLC 2020]. Further, NO has been proven to activate innate and adaptive responses of the immune system against tumors, depending primarily on its concentration (see e.g. Vannini et al. Redox Biol 6, 334-343, 2015; and Confino H etal. Nitric Oxide Lung Cancer Active Vaccination, NACLC 2020).

### SUMMARY OF THE INVENTION

According to an aspect of some embodiments of the present invention there is provided a method of producing a tumor vaccine, the method comprising:
(a) ex-vivo exposing a tumor sample to gaseous nitric oxide (gNO);
(b) suspending the tumor sample in a medium or buffer subsequent to the exposing, so as obtain tumor cells in suspension; and
(c) titrating a pH of the suspension to 6-8, thereby producing the tumor vaccine.

According to some embodiments of the invention, the method does not comprise culturing the tumor sample or cells following step (a) step (b) and/or step (c).

According to an aspect of some embodiments of the present invention there is provided a method of producing a tumor vaccine, the method comprising culturing a tumor sample in a medium comprising antibiotic at a concentration of at least 2 fold higher than the gold standard concentration for culturing primary cells of the same type as the tumor sample, thereby producing the tumor vaccine.

According to some embodiments of the invention, the method comprising ex-vivo exposing the tumor sample to gaseous nitric oxide (gNO).

According to some embodiments of the invention, exposing to the gNO is effected prior to the culturing.

According to some embodiments of the invention, exposing to the gNO is effected subsequent to the culturing.

According to an aspect of some embodiments of the present invention there is provided a method of producing a tumor vaccine, the method comprising:
(a) ex-vivo exposing a tumor sample to gaseous nitric oxide (gNO); and
(b) culturing the tumor sample in a medium comprising antibiotic at a concentration of at least 2 fold higher than the gold standard concentration for culturing primary cells of the same type as the tumor sample,

thereby producing the tumor vaccine.

According to some embodiments of the invention, the method comprising subjecting the tumor sample or the tumor vaccine to a preservation protocol.

According to some embodiments of the invention, the method comprising subjecting the tumor sample, the tumor cells or the tumor vaccine to a preservation protocol.

According to an aspect of some embodiments of the present invention there is provided a method of producing a tumor vaccine, the method comprising:
(a) ex-vivo exposing a tumor sample to gaseous nitric oxide (gNO); and
(b) subjecting the tumor sample to a preservation protocol, thereby producing the tumor vaccine.

According to some embodiments of the invention, the preservation protocol comprises freezing.

According to some embodiments of the invention, step (a) is effected prior to step (b).

According to some embodiments of the invention, step (a) is effected subsequent to step (b).

According to an aspect of some embodiments of the present invention there is provided a method of preventing and/or treating a tumor in a subject in need thereof, the method comprising administering to the subject a therapeutically effective amount of a tumor vaccine, wherein the tumor vaccine is obtainable by ex-vivo exposing a tumor sample to gaseous nitric oxide (gNO), and wherein the administering is effected at least twice, thereby preventing and/or treating the tumor in the subject.

According to an aspect of some embodiments of the present invention there is provided a method of inducing an immunological response to a tumor in a subject in need thereof, the method comprising administering to the subject a therapeutically effective amount of a tumor vaccine, wherein the tumor vaccine is obtainable by ex-vivo exposing a tumor sample to gaseous nitric oxide (gNO), and wherein the administering is effected at least twice, thereby inducing the immunological response to the tumor in the subject.

According to some embodiments of the invention, the method comprising ex-vivo exposing the tumor sample to the gNO.

According to some embodiments of the invention, the antibiotic comprises penicillin-streptomycin.

According to some embodiments of the invention, the medium comprises RPML According to some embodiments of the invention, the culturing is effected for at least 10 hours.

According to some embodiments of the invention, the culturing is effected for less than 24 hours.

According to some embodiments of the invention, the exposing to gNO is at a dose of 1000-1,000,000 ppm.

According to some embodiments of the invention, the exposing to gNO is at a dose of 25,000-250,000 ppm.

According to some embodiments of the invention, the exposing to gNO is at a dose of about 200,000 ppm.

According to some embodiments of the invention, the exposing to gNO is for a time period of from 1 second to 100 minutes.

According to some embodiments of the invention, the exposing to gNO is for a time period of 1-15 minutes.

According to some embodiments of the invention, the exposing to gNO is for a time period of 4-6 minutes.

According to some embodiments of the invention, the exposing to gNO is for a time period of 1-3 minutes.

According to some embodiments of the invention, the exposing to gNO is at a flow volume of from 0.1 liter per minute (LPM) to 10 LPM.

According to some embodiments of the invention, the exposing to gNO is at a flow volume of from 0.1 liter per minute (LPM) to 1 LPM.

According to some embodiments of the invention, the exposing to gNO is at a flow volume of 0.5-1.5 liter per minute (LPM).

According to some embodiments of the invention, the exposing to gNO is at a flow volume of 0.2-0.3 liter per minute (LPM).

According to some embodiments of the invention, the exposing to gNO is effected in the absence of medium or buffer.

According to some embodiments of the invention, the method comprising titrating pH of the tumor sample to pH 6-8 following the exposing to gNO.

According to some embodiments of the invention, the pH is 6.8-7.2.

According to some embodiments of the invention, the titrating the pH is effected by TRIS buffer, THAM buffer or NaOH base.

According to some embodiments of the invention, the method further comprising processing the tumor sample to obtain a tissue fragment, single cells, clumps and/or an extracted biomaterial.

According to some embodiments of the invention, the processing is effected subsequent to the exposing.

According to some embodiments of the invention, the processing is effected subsequent to the culturing.

According to some embodiments of the invention, the processing is effecting subsequent to the subjecting.

According to some embodiments of the invention, the tumor sample is a tissue sample.

According to some embodiments of the invention, the tumor tissue sample is 1-1000 mm³ According to some embodiments of the invention, a volume of the tumor tissue sample is 1-200 mm³.

According to some embodiments of the invention, the tumor sample is a dissociated cells sample.

According to some embodiments of the invention, the tumor sample is a single tumor cells or clumps sample.

According to some embodiments of the invention, a volume of the fragment is 1-150 mm³ According to some embodiments of the invention, the method further comprising contacting antigen presenting immune cells with the tumor sample or the tissue fragment, single cells, clumps and/or extracted biomaterial processed therefrom to thereby obtain immune cells presenting an antigenic biomaterial of the tumor sample.

According to some embodiments of the invention, the method comprising obtaining the tumor sample from a subject.

According to some embodiments of the invention, the tumor sample is obtained from multiple subjects.

According to some embodiments of the invention, the tumor sample comprises aprimary tumor.

According to some embodiments of the invention, the tumor sample comprises a secondary tumor.

According to some embodiments of the invention, the tumor sample comprises a combination of a primary and a secondary tumor.

According to some embodiments of the invention, the method further comprising determining viability and/or proliferation of the tumor vaccine.

According to some embodiments of the invention, tumor cells of the vaccine are non-proliferative and/or non-viable.

According to some embodiments of the invention, the method further comprising administering a therapeutically effective amount of the tumor vaccine to a subject in need thereof.

According to an aspect of some embodiments of the present invention there is provided a tumor vaccine obtainable by the method.

According to an aspect of some embodiments of the present invention there is provided a method of preventing and/or treating a tumor in a subject in need thereof, the method comprising administering to the subject a therapeutically effective amount of the tumor vaccine, thereby preventing and/or treating the tumor in the subject.

According to an aspect of some embodiments of the present invention there is provided a method of inducing an immunological response to a tumor in a subject in need thereof, the method comprising administering to the subject a therapeutically effective amount of the tumor vaccine, thereby inducing the immunological response to the tumor in the subject.

According to some embodiments of the invention, the therapeutically effective amount comprises 10-100 mg of the tumor sample or fragment processed therefrom According to some embodiments of the invention, the therapeutically effective amount comprises 0.1×10⁶-900×10⁶ cells.

According to some embodiments of the invention, the tumor sample and the tumor are of the same type.

According to some embodiments of the invention, the tumor sample is autologous to the subject.

According to some embodiments of the invention, the tumor sample is allogeneic to the subject.

According to some embodiments of the invention, the antigen presenting cells are autologous to the subject.

According to some embodiments of the invention, the subject in need thereof has not been diagnosed with a tumor.

According to some embodiments of the invention, the subject in need thereof has been diagnosed with a primary tumor.

According to some embodiments of the invention, the subject in need thereof has been diagnosed with a secondary tumor.

According to some embodiments of the invention, the secondary tumor is a recurrent tumor.

According to some embodiments of the invention, the secondary tumor is a metastasizing tumor.

According to some embodiments of the invention, the administering is effected once.

According to some embodiments of the invention, the administering is effected at least twice.

According to some embodiments of the invention, a time interval between the at least two administrations is at least one week.

According to some embodiments of the invention, a time interval between the at least two administrations is at least six months.

According to some embodiments of the invention, the administering is into the arm According to some embodiments of the invention, the administering is intra-tumorally.

According to some embodiments of the invention, the method further comprising administering to the subject an anti-cancer therapy.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof.

Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

FIG. 1 is a bar graph showing the effect of 50,000 ppm NO or air on CT-26 cells in-vitro.

FIG. 2 is a bar graph showing the effect of 150-50,000 ppm NO or air on 4T1 cells in-vitro.

FIG. 3 is a bar graph showing the effect of 4,000-50,000 ppm NO or air on A549 cells in-vitro.

FIG. 4 is a bar graph showing the effect of 4,000-50,000 ppm NO or air on PC-3 cells in-vitro.

FIG. 5 is a bar graph showing the effect of 10,000-25,000 ppm NO or air on B16.F10 cells in-vitro.

FIG. 6 is a bar graph showing the effect of 10,000-25,000 ppm NO or air on Panc02.03 cells in-vitro.

FIG. 7 is a bar graph showing the effect of 10,000-25,000 ppm NO or air on LLC1 cells in-vitro.

FIGS. 8A-C present the effect of 10,000-20,000 ppm NO or air on LLC1 cell death type in-vitro as measured in Annexin V—Propidium Iodide apoptosis-necrosis assay.

FIG. 9 presents comparative plots showing the effect of air or of gNO at 10,000 ppm or 50,000 ppm on CT26 tumor volume, in vivo.

FIGS. 10A-F are photographs depicting the effect of gNO at 10,000 ppm on a CT-26 tumor bearing mouse at 2 days before treatment, 1 minute after treatment, 9 days post treatment and 14 days post treatment, respectively (FIGS. 10A-D), and of the system used for delivering gNO by placing the tumor inside a container filled with gNO (FIG. 10E); and a tumor growth curve post-treatment with 10,000 ppm NO (FIG. 10F).

FIGS. 11A-C present tumor growth curves depicting the effect of gNO at 10,000-50,000 ppm on tumor volume in CT-26 tumor bearing mice, compared to air-treated CT-26 tumor bearing mouse as control (FIG. 11A), and photographs showing an exemplary system as used for intratumoral gNO delivery according to some embodiments of the present invention (FIG. 111B) and an exemplary system as used for gNO delivery via capping of a tumor according to some embodiments of the present invention (FIG. 11C).

FIGS. 12A-B present photographs showing the effect of air or 50,000 ppm gNO on CT-26 tumor cells ex-vivo, outside (outer layer of) the tumor tissue (FIG. 12A) and inside (inner layer of) the tumor tissue (FIG. 12B).

FIG. 13 are photographs depicting the effect of gNO at 25,000 ppm for 2 cycles of 15-minute treatment on two CT-26 tumor bearing mice before treatment (right photographs) and 44 days post treatment (left photographs).

FIG. 14 are photographs depicting the effect of gNO at 200,000 ppm for 2 seconds on a CT-26 tumor bearing mice before treatment (upper photographs) and 9 days post treatment (lower photograph).

FIGS. 15A-B present a schematic depiction of a challenge assay in which tumors of LLC1 tumor-bearing mice were treated with 50,000 ppm NO for 10 minutes, and 14 days post this gNO treatment, mice were re-inoculated with LLC1 cells (FIG. 15A), and a bar graph showing the percentage of challenge tumor take 9 days postLLC1 cancer cell re-inoculation (FIG. 15B). Naïve mice, inoculated with LLC1 for the first time, served as the control group.

FIGS. 16A-E present a schematic depiction of a challenge assay in which tumors of 4T1 tumor-bearing mice were treated with 50,000 ppm gNO or nitrogen gas for 10 minutes, and 14 days post this gNO treatment, mice were re-inoculated with 4T1 cells (FIG. 16A), comparative plots showing the average primary tumor volume after gaseous nitrogen or gNO treatment (FIG. 16B), comparative plots showing the average challenge tumor volume 10 days post re-inoculation of cancer cells (FIG. 16C), a bar graph showing the percentage of challenge tumor take 10 days post 4T1 cancer cells re-inoculation with Naïve mice, inoculated with 4T1 for the first time being the control group (FIG. 16D), and a bar graph showing the number of mice that developed a challenge tumor 10 days post cancer cell re-inoculation in each treatment or control group.

FIGS. 17A-B present a schematic depiction of a challenge assay in which tumors of CT26 tumor-bearing mice were treated with 25,000 ppm gNO for two 15 minute-cycles, 50,000 ppm gNO for 10 minutes or 200,000 ppm gNO for 2-35 seconds, and up to 14 days post this gNO treatment, mice were re-inoculated with CT26 cells (FIG. 17A), and a bar graph showing the percentage of CT26 challenge tumor take after re-inoculation (FIG. 17B). Naïve mice, inoculated with CT26 for the first time, served as the control group.

FIGS. 18A-C present a schematic depiction of a challenge assay in which splenocytes extracted from CT26 gNO-immunized mice where inoculated to naïve mice depict (FIG. 18A), a bar graph showing the percentage of CT26 tumor cells take when mixed with splenocytes extracted from CT26 immunized mice prior to inoculation to naïve mice (FIG. 18B) and a bar graph showing the number of mice that developed a tumor after inoculation of CT26 cells together with splenocytes from an immunized mouse (FIG. 18C).

FIG. 19 is a bar graph showing the percentage of CT26 cells viability after incubation with splenocytes extracted from a CT26 gNO-immunized mouse.

FIG. 20 is a bar graph showing the effect of combined 20,000 ppm or 50,000 ppm gNO and surgery on CT26 tumor recurrence.

FIGS. 21A-D present a schematic depiction of a challenge assay in which tumors of CT26 tumor-bearing mice were treated with 20,000 ppm or 50,000 ppm gNO for 5 minutes and 14 days post this treatment, all tumors were excised and mice were re-inoculated with CT26 cells (FIG. 21A), a bar graph showing the percentage of challenge tumor take in each group compared to Naïve mice (FIG. 21B), comparative plots showing the tumor volume up to 21 days post re-inoculation (Challenge) (FIG. 21C), and a bar graph showing survival of mice in each group (FIG. 21D).

FIG. 22 illustrates an exemplary system for exposing the patient's tumor to gNO in a container, according to some embodiments of the present invention.

FIG. 23 illustrates an exemplary intra-tumoral delivery system of gNO by a needle, according to some embodiments of the present invention.

FIG. 24 illustrates an exemplary system for spraying nitric oxide, according to some embodiments of the present invention.

FIG. 25 illustrates an exemplary nitric oxide delivery to the tumor by capping of an outgrowth, according to some embodiments of the present invention.

FIG. 26 illustrates an exemplary laparoscope-guided gNO administration and evacuation from the tumor site, according to some embodiments of the present invention.

FIG. 27 illustrates an exemplary double intra-tumoral channel system for administering and removal of gNO, according to some embodiments of the present invention.

FIG. 28 illustrates an exemplary needle-capping gNO exposure system providing both intra- and out-tumor gas, according to some embodiments of the present invention.

FIG. 29 illustrates an exemplary perforated scalpel for tumor excision with gNO delivery, according to some embodiments of the present invention.

FIG. 30 illustrates an exemplary full-body chemical hood with gNO and NOx vacuuming, according to some embodiments of the present invention.

FIG. 31 illustrates an exemplary chemical vapor for local gas extraction which is located above the tumor, according to some embodiments of the present invention.

FIG. 32 illustrates an exemplary one-way gas delivering valve, according to some embodiments of the present invention.

FIG. 33 illustrates an exemplary gaseous NO delivery and scavenging patch, according to some embodiments of the present invention.

FIG. 34 illustrates an exemplary gNO perforated spray needle, according to some embodiments of the present invention.

FIGS. 35A-C illustrate an exemplary tumor excision procedure accompanied by gNO release before (FIG. 35A) during (FIG. 35B) and after (FIG. 35C) surgery, according to some embodiments of the present invention.

FIG. 36 illustrate an exemplary imaging guided gNO based treatment, according to some embodiments of the present invention.

FIGS. 37A-B present a schematic depiction of a challenge assay in which tumors of CT26 tumor-bearing mice were treated with 20,000 ppm gNO, air or nitrogen for two 15-min cycles and 14 days post this treatment, mice were re-inoculated with CT26 cells (FIG. 37A), a bar graph showing the percentage of tumor-free mice, without visible primary and challenge tumors in the control group, the air or nitrogen-treated group and gNO-treated group (FIG. 37B).

FIG. 38 presents a schematic illustration of an exemplary process of preparing an ex-vivo NO-based personalized cancer vaccine.

FIG. 39 is a schematic illustration of an exemplary system for delivery of gas to a tissue of a subject, according to some embodiments of the present invention.

FIG. 40 is a flowchart diagram illustrating a method suitable for delivery of a gas to a tissue, according to some embodiments of the present invention.

FIG. 41 is a schematic illustration of the study protocol described in Example 25 hereinbelow.

FIG. 42 is a graph demonstrating resistance of mice to tumors following implantation of ex-vivo treated tumor sample. CT26 tumor were excised from mice, the skin was removed from the tumors and the tumors were incubated overnight at 37° c. and 5% CO₂ in RPMI-based cell culture media supplemented with 2% Penicillin-Streptomycin and 10% fetal bovine serum. Following incubation, treated CT26 tumors were re-implanted to the left flank of each mouse. Twenty-one days post re-implanting, mice were re-challenged using 500,000 CT26. Shown the percentage of challenge tumor take 30 days post CT26 cancer cell re-inoculation. Naïve mice, inoculated with CT26 cells for the first time, served as the control group.

FIG. 43 is a schematic illustration of the study protocol described in Example 26 hereinbelow.

### DESCRIPTION OF SPECIFIC EMBODIMENTS OF THE INVENTION

The present invention, in some embodiments thereof, relates to methods of producing tumor vaccine and uses thereof.

Embodiments of the present invention relate to methods of producing a tumor vaccine.

Hence, according to an aspect of the present invention, there is provided a method of producing a tumor vaccine, the method comprising:
(a) ex-vivo exposing a tumor sample to gaseous nitric oxide (gNO);
(b) suspending said tumor sample in a medium or buffer subsequent to said exposing, so as obtain tumor cells in suspension; and
(c) titrating a pH of said suspension to 6-8, thereby producing the tumor vaccine.

According to an additional or an alternative aspect of the present invention, there is provided a method of producing a tumor vaccine, the method comprising culturing a tumor sample in a medium comprising antibiotic at a concentration of at least 2 fold higher than the gold standard concentration for culturing primary cells of the same type as said tumor sample, thereby producing the tumor vaccine.

According to an additional or an alternative aspect of the present invention, there is provided a method of producing a tumor vaccine, the method comprising:
(a) ex-vivo exposing a tumor sample to gaseous nitric oxide (gNO); and
(b) culturing said tumor sample in a medium comprising antibiotic at a concentration of at least 2 fold higher than the gold standard concentration for culturing primary cells of the same type as said tumor sample, thereby producing the tumor vaccine.

According to an additional or an alternative aspect of the present invention, there is provided a method of producing a tumor vaccine, the method comprising:
(a) ex-vivo exposing a tumor sample to gaseous nitric oxide (gNO); and
(b) subjecting said tumor sample to a preservation protocol, thereby producing the tumor vaccine.

Specific embodiments of the present invention further suggest use of the obtained tumor vaccine for inducing an immunological response to a tumor in a subject in need thereof or preventing and/or treating a tumor in a subject in need, as further described in details hereinbelow.

Medium comprising antibiotic The tumor sample of some embodiments of the invention is cultured prior to, during or following the method.

According to specific embodiments, the method comprising culturing the tumor sample.

According to specific embodiment, the culturing is effected prior to the exposing to gNO.

According to specific embodiment, the culturing is effected subsequent to the exposing to gNO.

According to specific embodiments, the culturing is effected subsequent to the titrating.

The tumor sample of some embodiments of the invention is cultured in a medium comprising antibiotic at a concentration of at least 2 fold higher than the gold standard concentration for culturing primary cells of the same type as the tumor sample.

The culture medium used by some embodiments can be a water-based medium which includes a combination of substances such as salts, nutrients and minerals and may further be supplemented with vitamins, antibiotics, anti-fungal agents (e.g. amphotericin B), amino acids [e.g. L-glutamine or NEAA (non-essential amino acids)], nucleic acids and/or proteins such as cytokines, growth factors and hormones.

Non-limiting Examples of media that can be used with specific embodiments of the invention include RPMI (can be obtained from e.g. Gibco-Invitrogen Corporation products), DMEM (can be obtained from e.g. Biological Industries), EMEM (can be obtained from e.g. Sigma-Aldrich), MEM (can be obtained from e.g. Sigma-Aldrich).

According to specific embodiments, the medium comprises RPMI.

Preferably, all ingredients included in the culture medium of the present invention are substantially pure, with a tissue culture grade.

The skilled artisan would know to select the culture medium for each type of tumor sample contemplated.

According to specific embodiments of the invention, the culture medium comprises serum e.g. fetal calf serum (FCS, can be obtained e.g. from Gibco-Invitrogen Corporation products).

According to specific embodiments, the culture medium is devoid of serum.

According to some embodiments of the invention, the culture medium is devoid of any animal contaminants, i.e., animal cells, fluid or pathogens (e.g., viruses infecting animal cells), i.e., being xeno-free.

Non-limiting Examples of antibiotics that can be used with specific embodiments of the invention include penicillin-streptomycin, penicillin-streptomycin-amphotericin, penicillin-streptomycin-nystatin, penicillin, streptomycin Sulfate, Gentamycin Sulfate, Penicillin-Streptomycin-Neomycin, Neomycin Sulfate, Kanamycin Sulfate, Amphotericin, Kanamycin Sulfate, Penicillin G Sodium, Nystatatin, gentamicin.

According to specific embodiments, the antibiotic comprises penicillin-streptomycin.

The skilled artisan knows how to handle antibiotics in terms of storage, stability, buffer, temperature at the like.

Gold standard relates to a regulatory agency (e.g., FDA) approved dose for culturing cells used for cell therapy.

Non-limiting Examples of gold standard concentrations of antibiotics in culture media include 1% penicillin-streptomycin (i.e. 100 U/ml penicillin and 100 μg/ml streptomycin), 1% Nystatin, 0.1-1% Amphotericin B Solution.

According to specific embodiments, the tumor sample is cultured in a medium comprising at least 2% penicillin-streptomycin (i.e. 200 U/ml penicillin and 200 μg/ml streptomycin).

According to specific embodiments, the tumor sample is cultured in a medium comprising 2% penicillin-streptomycin (i.e. 200 U/ml penicillin and 200 μg/ml streptomycin).

According to specific embodiments, the culturing is effected for at least 2, at least 3, at least 4, at least 5, at least 6, at least 7, at least 8, at least 9, at least 10, at least 12 hours.

According to specific embodiments, the culturing is effected for at least 10 hours. According to specific embodiments, the culturing is effected for less than 72, less the 48, less than 24, less than 20 hours.

According to specific embodiments, the culturing is effected for less than 24 hours.

According to specific embodiments, the culturing is effected for 6-24 hours, 6-20 hours, 8-20 hours, 8-16 hours, 10-16 hours or 10-12 hours.

According to specific embodiments, the culturing is effected overnight.

According to some of any of the embodiments described herein, in cases where the tumor sample is covered by skin, peritoneum, crust or any other thick layer, this layer can be removed prior to or during culturing of the tumor.

According to other specific embodiments, the method does not comprise culturing the tumor sample following the ex-vivo exposing to gNO, following the suspending and/or following the titration.

According to other specific embodiments, the method does not comprise culturing the tumor sample following the ex-vivo exposure to gNO.

According to other specific embodiments, the method does not comprise culturing the tumor cells following suspension of the tumor sample.

According to other specific embodiments, the method does not comprise culturing the tumor cells following the titration.

According to specific embodiments, the tumor sample or any cell preparation derived therefrom is not subjected to culturing prior to, during and/or following the method.

Exposure to gaseous nitric oxide (gNO): Specific embodiments of the present invention provides methods of producing a tumor vaccine which employ ex-vivo exposure to gNO, as described in further detail hereinunder.

According to specific embodiments, the exposing to gNO is effected prior to or subsequent to the culturing.

According to specific embodiments, the exposing to gNO is effected with high dose gNO.

High dose gNO is defined as the delivery of gNO in a preferably inert gas such as nitrogen at a concentration of between about 1,000 and 1,000,000 ppm, as is described in further detail hereinafter.

The methods of delivering gNO may include administration of gNO in a continuous or pulsed manner.

In some embodiments, exposing to gNO is effected intratumorally, by applying gNO directly into the tumor sample. In some embodiments, exposing to gNO is effected by applying gNO to the surface of the tumor sample, for example, by contacting the tumor's surface with gNO. In some embodiments, exposing to gNO is effected by bringing gNO in close vicinity to the tumor sample, for example, directly or up to 2 cm, or up to 1 cm, from at least one and preferably all of the tumor surfaces.

In some of any of the embodiments described herein, exposing to gNO is effected intratumorally, such that gNO is injected or otherwise delivered into the tumor sample.

In some of any of the embodiments described herein, exposing to gNO is effected by spraying or otherwise applying gNO onto at least one of the tumor's surface. At least some of the gNO then enters into the tumor sample via, e.g., diffusion.

In some of any of the embodiments described herein, exposing to gNO is effected by exposing the tumor sample to gNO in a container, such that gNO is contacted with the tumor and enters into the tumor via e.g., diffusion. The container can be open or closed and can be sized to conform to the contours of the tumor sample.

Thus, according to specific embodiments, exposure to the gNO can be performed, for example, by placing the tumor sample in a closed container or hood, and introducing gNO to the closed container or hood, or directly into or on the tumor sample, for example, by spraying or injecting, using any of the systems, devices, configurations and modes of administration as described herein in any of the respective embodiments and any combination thereof, when applied or adjusted for application ex vivo.

According to specific embodiments, the tumor sample is are plated on a culture plate placed inside the container or hood.

These and other embodiments relating to methodologies and systems for exposing to gNO are described in further detail hereinunder.

According to some of any of the embodiments described herein exposing to gNO is with a high dose of gNO, as described herein. These embodiments are also referred to herein as “high dose exposure of gNO”, “high dose gNO treatment”, and grammatical diversions of the foregoing, or simply as “high dose gNO”.

In some of any of the embodiments described herein, the high dose of gNO is reflected by its relatively high concentration in the total amount of gas provided to the tumor sample, and is presented by ppm (part per million) units.

In some of any of the embodiments described herein, the high dose of gNO is presented as its fraction, in ppm units, in the gas carrier. The gas carrier can be air, and preferably an inert gas such as nitrogen or argon, preferably nitrogen.

In some of any of the embodiments described herein, the high dose of gNO is reflected by the mass of gNO that is provided to the tumor sample, per a volumetric unit of the tumor sample. According to some of any of the embodiments described herein, the gNO is provided, as described herein in any of the respective embodiments, at a concentration of from about 1,000 ppm to about 1,000,000 ppm (0.1% to 100%), including any intermediate values and subranges therebetween, for example, from about 1,000 ppm to about 200,000 ppm, or from about 1,000 ppm to about 100,000 ppm, preferably from about 10,000 ppm to about 500,000 ppm, or from about 10,000 ppm to about 200,000 ppm, or from about 10,000 ppm to about 100,000 ppm, or from about 20,000 ppm to about 100,000 ppm, or from 25,000-250,000 ppm, or from about 25,000 ppm to about 100,000 ppm, or from about 25,000 ppm to about 75,000 ppm, or from about 10,000 ppm to about 50,000 ppm, or from about 50,000 ppm to about 100,000 ppm, including any intermediate values and subranges between any of the foregoing, or is about 50,000 ppm, or about 200,000 ppm.

In some of any of the embodiments described herein in the context of high dose of gNO, the high dose can be obtained by exposing to gNO at a dose of at least 10,000 ppm, or at least 20,000 ppm, or at least 50,000 ppm, and optionally up to about 1,000,000 ppm.

In exemplary embodiments, exposing to gNO is effected as described herein in any of the respective embodiments, at a concentration of about 200,000 ppm.

In exemplary embodiments, exposing to gNO is effected as described herein in any of the respective embodiments, at a concentration of about 50,000 ppm.

In exemplary embodiments, the exposing to gNO is effected, as described herein in any of the respective embodiments, at a concentration of about 25,000 ppm. In exemplary embodiments, the exposing to gNO is effected, as described herein in any of the respective embodiments, at a concentration of about 20,000 ppm. In exemplary embodiments, the exposing to gNO is effected, as described herein in any of the respective embodiments, at a concentration of about 10,000 ppm. In exemplary embodiments, the exposing to gNO is effected, as described herein in any of the respective embodiments, at a concentration of about 100,000 ppm. In exemplary embodiments, the exposing to gNO is effected, as described herein in any of the respective embodiments, at a concentration of from about 100,000 ppm to about 200,000 ppm, or from about 200,000 ppm to about 500,000 ppm, or from about 500,000 ppm to about 1,000,000 ppm or from about 50,000 ppm or about 100,000 ppm or about 200,000 ppm or about 500,000 ppm or about 1,000,000 ppm.

According to some of any of the embodiments described herein, the exposing to gNO is effected, as described herein in any of the respective embodiments, at a volumetric flow rate of from about 0.00001 LPM to about 10 LPM, preferably from about 0.0001 LPM and about 1 LPM, or from about 0.001 LPM and 0.5 LPM, including any intermediate values and subranges therebetween. For example, the volumetric flow rate can be from about 0.001 LPM to about 0.01 LPM, or from about 0.01 LPM to about 0.1 LPM, or from about 0.1 LPM to about 0.25 LPM, or from about 0.25 LPM to about 0.5 LPM, or from about 0.5 LPM to about 1 LPM, or from about 0.2 LPM to about 0.3 LPM, or from about 1 LPM to about 2 LPM, or from about 2 LPM to about 3 LPM, or from about 3 LPM to about 4 LPM, or from about 4 LPM to about 5 LPM, or from about 5 LPM to about 6 LPM, or from about 7 LPM to about 8 LPM, or from about 8 LPM to about 9 LPM, or from about 9 LPM to about 10 LPM, including any intermediate values and subranges therebetween, or it can be, for example, about 0.0001 LPM, or about 0.001 LPM, or about 0.01 LPM, or about 0.1 LPM, or about 1 LPM or about 10 LPM.

According to specific embodiments, the exposing to gNO is effected at a volumetric flow rate of from about 0.1 LPM to about 10 LPM.

According to specific embodiments, the exposing to gNO is effected at a volumetric flow rate of from about 0.1 LPM to about 1 LPM.

According to specific embodiments, the exposing to gNO is effected at a volumetric flow rate of 0.5-1.5 LPM.

According to specific embodiments, the exposing to gNO is effected at a volumetric flow rate of 0.2-0.3 LPM.

According to some of any of the embodiments described herein, the exposing to gNO is effected for a time period that ranges from about 0.1 seconds to about 10 hours, per administration, including any intermediate values and subranges therebetween. For example, the time period can be from about 0.1 second to about 1 hour, or from about 1 second to about 100 minutes, or from about 1 second to about 10 minutes, or from about 1 minute to about 10 minutes, or from about 10 seconds to about 10 minutes, or from about 0.1 second to about 10 minutes, or from about 30 seconds to about 3 minutes, or from about 1 minute to 15 minutes, or from about 1 minute to about 30 minutes, or from about 4 minutes to about 6 minutes, or from about 1 minute to about 3 minutes or from about 10 minutes to about 60 minutes, or from about 60 minutes to about 180 minutes, or from about 180 minutes to about 600 minutes, including any intermediate values and subranges between any of foregoing, or it can be about 30 seconds, about 2 minutes, about 5 minutes, about 10 minutes, about 30 minutes, or about 60 minutes.

In some of any of the embodiments related to methods and uses as described herein exposing to gNO is effected, as described herein in any of the respective embodiments, at a dose of from about 1,000 ppm to about 1,000,000 ppm for a time period of from about 1 second to about 60 minutes at a volumetric flow (flow volume) of from about 0.00001 liter per minute (LPM) to about 1 LPM, including any intermediate values and subranges between any of the foregoing. In some of any of the embodiments related to methods and uses as described herein exposing to gNO is effected, as described herein in any of the respective embodiments, at a dose of at least 10,000 ppm, and optionally up to about 1,000,000, or up to about 500,000 ppm, or up to about 200,000 ppm, or up to about 100,000 ppm, for a time period of from about 1 second to about 60 minutes at a volumetric flow (flow volume) of from about 0.0001 liter per minute (LPM) to about 1 LPM, including any intermediate values and subranges between any of the foregoing.

In some of any of the embodiments described herein exposing to gNO is effected at a dose of at least 10,000 ppm, or at least 20,000 ppm, or at least 50,000 ppm, and optionally up to about 1,000,000 ppm, for a time period of at least 1 second, or at least 10 seconds, or at least 30 seconds, or at least 1 minute, and optionally up to about 60 minutes, at a volumetric flow rate (flow volume) of at least 0.0001 liter per minute (LPM), or at least 0.001 LPM, or at least 0.01 LPM, and optionally up to about 1 LPM, including any intermediate values and subranges between any of the foregoing.

Alternatively or in addition, in any of the methods and uses described herein exposing to gNO is effected in ranges from about 0.1 mg to about 300 mg, per cm³ tumor sample, per exposure.

According to specific embodiments, the parameters of the gNO concentration (ppm), volumetric flow rate (LPM) and time to achieve a desired mass of gNO, and vice versa, the gNO mass achieved by exposing to gNO at a concentration, volumetric flow and time, can be calculated using the known ideal gas equation, PV=nRT, wherein P is the pressure, V is the volume, n is the number of moles, R is the gas constant and T is the temperature.

More specifically, these relations can be calculated or converted one to the other using the following equations:

$$\begin{matrix}1 ) X = y \times 1 {0}^{- 6} \\ 2 ) V = \dot{V} \times t \\ 3 ) n = \frac{V \times 1 {0}^{- 3} \times 101325}{8.314 \times 298} \\ 4 ) m = n \times X \times 30.01 \times 1 {0}^{3} \\ 5 ) m = ( \frac{( \dot{V} \times t ) \times 1 {0}^{- 3} \times 101325}{8.314 \times 298} ) \times ( y \times 1 {0}^{- 6} ) \times 30.01 \times 1 {0}^{3}\end{matrix}$$

y is the concentration in ppm units;

X is the concentration in molar fraction units, and equation 1 presents the relation between molar fraction and ppm (y); V is the volume, V is the volumetric flow in LPM and t is the time in minutes, and equation 2 presents the relation between Volume, volumetric flow rate and time; Equation 3 is the Ideal gas equation, and the 10⁻³ factor is added for transformation from Liters to m³.

Equation 4 presents the relation between the mass (m) the mole number (n), the molar fraction X and Nitric Oxide molar mass (30.01 grams/mol); and Equation 5 is a combination of equations 1-4 into a single equation, reflecting the relation between the mass of gNO and the molar fraction, volumetric flow and time. The 10³ is for obtaining the mass m on a milligram (mg) scale.

Thus, for example, at a volumetric flow of 0.1 LPM, time of 1 minute and concentration of 50,000 ppm, using equation 5 above, about 6.1-6.2 mg gNO is administered.

According to some of any of the embodiments described herein, the exposing to gNO is effected, as described herein in any of the respective embodiments, at a concentration of from about 1,000 ppm to about 1,000,000 ppm (0.1% to 100%), preferably from about 10,000 ppm to is about 500,000 ppm, or from about 10,000 ppm to about 100,000 ppm, or at about 50,000 ppm; at a volumetric flow rate of from about 0.0001 LPM to about 10 LPM, preferably from about 0.001 LPM and 1 LPM; and during a time period that ranges from about 1 second to about 30 minutes, per exposure.

According to some of any of the embodiments described herein, the exposing to gNO is effected, as described herein in any of the respective embodiments, at a concentration (dose) of from about 20,000 ppm to about 100,000 ppm or from about 20,000 ppm to about 50,000 ppm; for a time period that ranges from about 30 seconds to about 10 minutes; at a volumetric flow (flow volume) of from about 0.001 LPM to about 0.5 LPM, per exposure.

According to some of any of the embodiments described herein, the exposing to gNO is effected, as described herein in any of the respective embodiments, in an amount of no more 1 mg gNO per 100 mm³ tumor sample volume, per exposure.

According to some of any of the embodiments described herein, the exposing to gNO is effected, as described herein in any of the respective embodiments, in an amount of about 250 mg per cm³ tumor sample, per exposure.

According to some of any of the embodiments described herein, exposing to gNO is effected, as described herein in any of the respective embodiments, in an amount of from about 0.1 to about 10 mg per a tumor sample of 20 mm³ or less, including any intermediate values and subranges therebetween.

According to some of any of the embodiments described herein, the exposing to gNO is effected, as described herein in any of the respective embodiments, in an amount of from about 0.1 to about 300 mg including any intermediate values and subranges therebetween, per 1 cm³ tumor sample, per exposure. For example, the exposing to gNO is effected in an amount of from about 0.1 mg to about 250 mg, or from 0.1 mg to about 100 mg, or from 1 mg to about 50 mg, or from about 1 mg to about 100 mg, or from about 1 mg to about 300 mg, of from about 50 mg to about 100 mg, or from about 50 mg to about 300 mg, or from about 100 mg to about 150 mg, or from about 100 mg to about 300 mg, or from about 10 mg to about 100 mg, or of from about 10 mg to about 250 mg, or from about 0.1 mg to about 10 mg, or from about 10 mg to 200 mg, including any intermediate values and subranges of any of the foregoing, per 1 cm³ tumor sample, per exposure.

According to some of any of the embodiments described herein, the exposing to gNO is effected, as described herein in any of the respective embodiments, to a tumor sample having a volume of up to 20 mm³, from about 0.001 mg to about 10 mg, or from about 0.01 mg to about 20 mg, or from about 0.01 mg to about 2 mg, or from about 0.1 mg to about 1.0 mg, or from about 0.2 mg to about 0.8 mg, including any intermediate values and subranges between any of the foregoing, per exposure.

For any of the embodiments described herein for ex-vivo exposing to gNO, the exposing can be either continuous or pulsed, such that for each exposure, the indicated dose of gNO is administered either continuously or in a pulsed manner. When the high dose is referred to in ppm units, each pulse is at the indicated high dose concentration, as described herein in any of the respective embodiments. When the high dose is referred to as the total mass per administration, the indicated dose is divided into pulses.

According to some of any of the embodiments described herein, gNO is pulsed from about 2 to about 50 times, or from about 2 to about 30 times, or from about 2 to about 20 times, or from about 2 to about 15 times, or from about 5 to about 15 times, including any intermediate values and subranges therebetween, or about 10 times, per exposure.

According to some of any of the embodiments described herein, each pulse is between about 1,000 ppm and about 1,000,000 ppm, or between 4000 ppm and about 1,000,000 ppm gNO, or between 10,000 ppm and about 1,000,000 ppm gNO, at a volumetric flow (flow volume) of from about 0.00001 LPM to about 0.5 LPM, wherein each pulse is, independently, between about 0.1 second and about 10 minutes per pulse with a break of from about 0.1 second to about 10 minutes between pulses.

According to some of any of the embodiments described herein, each pulse of gNO is, independently, from about 10 seconds per pulse to about 45 seconds per pulse, including any intermediate values and subranges therebetween.

According to some of any of the embodiments described herein, each pulse of gNO is about 30 seconds per pulse.

According to some of any of the embodiments described herein, gNO is not administered between pulses and the time between each two pulses is, independently, from about 1 second to about 300 seconds, or from about 1 second to about 200 seconds, or from about 1 second to about 100 seconds, or from about 1 second to about 50 seconds, or from about 10 seconds to about 50 seconds, including any intermediate values and subranges therebetween, or is about 20 seconds.

According to some of any of these embodiments, the ratio between the time of gNO pulsed administration and the resting time between pulses ranges from 1:2 to 1:5. For example, for each pulse of gNO administration during 5 seconds, a following resting time is independently from 10 to 50 seconds. Preferably, the gNO is pulsed such that about 33% of the time gNO is delivered and 66% of the time is resting or waiting time between pulses.

According to some of any of the embodiments described herein, the exposing to gNO is effected, as described herein in any of the respective embodiments, at two or more administration sites in or on the tumor sample (depending in the administration mode). In some of these embodiments, the distance between the two administration sites is, independently, from about 2.5 mm to about 1 cm, or from about 0.25 cm to about 0.5 cm, including any intermediate values and subranges therebetween.

When gNO is administered to two or more sites of the tumor sample, each administration is at the ppm dose or mass amount indicated herein in any of the respective embodiments, or, the total mass (amount) administered to all tumor sites is as indicated herein in any of the respective embodiments.

According to some of any of the embodiments described herein, the method further comprises scavenging excess gNO from the one or more administration sites, as described in further detail hereinunder. In exemplary embodiments, the scavenging comprises applying a reduced pressure (vacuum) around the administration site(s).

According to some of any of the embodiments described herein exposure to gNO is performed one or more times per a treatment session.

In some embodiments, it is performed once during a treatment session. In some embodiments, it is performed twice, trice or more times during a treatment session.

According to some of any of the embodiments described herein, in cases where the tumor sample is covered by skin, peritoneum, crust or any other thick layer, this layer can be removed prior to or during exposure of the tumor sample to the gNO.

In any of the embodiments described herein, the gNO may be provided by an external source, for example, a reservoir of gNO or a chemical generator of gNO. In some embodiments, gNO is provided by a reservoir of gNO, preferably of a small volume of, for example, a single administration dosage (that is, the dose of gNO used per a single administration, as described herein in any of the respective embodiments). Such reservoirs are described in further detail hereinunder.

The gNO is preferably of medical purity, that is, preferably at least about 95%, more preferably at least about 99%, and even more preferably at least about 99.5% pure gNO. The gNO is preferably provided as a mixture of gNO and other gases, such as air, nitrogen, oxygen, and so forth, preferably an inert gas such as, for example, nitrogen, and its ppm concentration is within the gas it is mixed with.

According to specific embodiments, the tumor sample is exposed to a cytotoxic dose of gNO.

According to specific embodiments, exposing to gNO is effected in the absence of medium or buffer. Hence, according to specific embodiments, the method comprising removing from the tumor sample a medium or a buffer prior to exposing to gNO. According to specific embodiments, a medium or buffer is added to the tumor sample following the exposure to gNO.

According to specific embodiments, the method comprises dissociating single cells or clumps from the tumor sample prior to addition of the medium or buffer.

According to specific embodiments, the method comprises dissociating single cells or clumps from the tumor sample following addition of the medium or buffer.

Methods of dissociating cells are well known in the art and include, but not limited to, mechanical or chemical dissociation and/or enzymatic digestion, as further described herein below.

Non-limiting examples of buffers that can be used with specific embodiments of the invention include Dulbecco's Phosphate Buffered Saline (PBS), Hanks' Balanced Salt Solution (HBSS).

According to specific embodiments, the pH of the tumor sample or suspension is titrated following the exposure to gNO. Thus, for example, the pH of the tumor sample or suspension is titrated according to specific embodiments to pH 6-8, following the exposure to gNO.

According to specific the pH of the tumor sample or suspension is titrated to pH of about 7 following the exposure to gNO.

According to specific the pH of the tumor sample or suspension is titrated to pH of 6.8-7.2 following the exposure to gNO.

Methods of titrating pH are well known in the art. Non-limiting examples of titration buffers include tris(hydroxymethyl)aminomethane, also known as THAM or TRIS, Lithium hydroxide LiOH, Sodium hydroxide NaOH, Potassium hydroxide KOH, Rubidium hydroxide RbOH, Cesium hydroxide CsOH, Magnesium hydroxide Mg(OH) 2, Calcium hydroxide Ca(OH) 2, Strontium hydroxide Sr(OH) 2, Barium hydroxide Ba(OH) 2, Tetramethylammonium hydroxide N(CH3) 4OH.

According to specific embodiments, the pH is titrated by TRIS buffer, THAM buffer or NaOH base.

Systems and modes of administration of gNO: Embodiments of the present invention further relate to a system, which is also referred to herein interchangeably as “device”, which is configured for exposing to gNO to a tumor sample as described herein in any of the respective embodiments. Such a system is also referred to herein as a delivery system.

Generally, but not obligatory, such a system can include a pressure regulator, a flow meter, optionally an exposure box or container, one or more delivery lines, which are optionally terminated by or connected to a delivery device or configuration through which the gNO is administered and further optionally, a NO and/or NOx (as defined hereinunder) detector. Purging the gNO delivery system with an inert gas, such as nitrogen, may be desired.

The volume and/or flow rate of the administered (delivered) gas can be regulated by a digital flow controller, designed to deliver low volumes or flow rates of gas, of less than 0.1 LPM per cm³ of tissue, in accordance with any of the respective embodiments as described herein. Purging of the gNO delivery system, including purging the pressure regulator, flow meter and delivery lines can be performed before and/or after exposure to gNO. The gas purge can preferably last at least 1 minute or until the NO and NOx (as described below) detectors read no signal. In exemplary embodiments, nitrogen is used as the purging gas at a flow rate of at least 0.5 LPM.

The delivery device as described herein is meant to describe a component or configuration of the delivery system through which the gas exits the delivery system and contacts the tumor sample.

According to some of any of the embodiments described herein, exposing to gNO to a tumor sample as described herein in any of the respective embodiments can be accomplished by delivery device means such as one or more needles, including, for example, perforated spray needles, non-perforated and non-spray needles, umbrella needles, or other needles. The needles can optionally be nano-sized, micron-sized or macro-sized needles (having a diameter of 1 mm or higher). Other delivery devices are described hereinunder. Embodiments in which needles are used are typically used when the gNO is administered intra-tumorally, e.g., by intratumoral injection.

In some of any of the embodiments described herein in the context of a delivery system, the opening from which the gNO is delivered can be adjusted to the size of the tumor sample. In some embodiments, the opening does not exceed the size of the tumor.

The methods of exposing to gNO to a tumor sample can include contacting at least a portion of the tumor sample with the gaseous nitric oxide, and subsequently removing gaseous nitric oxide and NOx gas molecules from the treated site during or after the administration step. Vacuuming the gas can be done in a pulsed or continuous manner, preferably synchronized with the gNO mode of administration.

A delivery system can include a differently-sized chemical hood designed to evacuate excessive gNO or NOx during treatment, in a pulsed or continuous manner, preferably synchronized with the gNO mode of administration.

The delivery system can include an evacuation cylinder, which is connected directly to a regulator of the gNO tank. To purge the regulator safely, the evacuation cylinder can be filled with a gas accumulated in the regulator.

Another embodiment of controlling high dose gNO includes the use of a one-way valve where disconnecting the regulator or flow meter from the cylinder locks the valve, thereby preventing gas release from a gas tank.

According to some of any of the embodiments described herein, the methods, uses and delivery systems as described herein utilize a gNO cylinder, optionally equipped with a gas regulator, and one or more valves, and further optionally, the cylinder further comprises a delivery device for executing the local administration. The delivery device is in fluid communication with the cylinder, preferably via the valves and gas regulator (e.g., flow controller). Alternatively, the cylinder comprises means to connect the delivery device to the cylinder, to obtain fluid communication therebetween. The delivery device can be, for example, a scope with an annular-shape or a needle or a device configured for spraying the tumor, or else, as described in further detail hereinunder.

According to some of any of the embodiments described herein, the gNO cylinder is a miniature or at least portable cylinder.

According to some embodiments, the cylinder is of a volume of less than 1 liter, or less than 0.8 liter, or less than 0.75 liter, or less than 0.5 liter, or less than 0.3 liter.

The cylinder is preferably under low pressure, such as less than about 40 bar (about 600 psi). Thus, the cylinder can deliver about 10 liters of high concentration gNO. A regulator can optionally limit output pressure to about 50 psi, for example, and can be connected to an emergency on/off valve. The gas flow, in a case of, for example, a delivery system configured for injection, can be less than about 0.1 LPM or about 0.05 LPM. The use of such low flow rates during exposure to gNO can assist in limiting the exposure to the tumor or cancerous cells.

According to some of any of the embodiments described herein, the methods and uses involve scavenging gNO and optionally other gases, and the scavenging can be performed by applying vacuum so as to remove gNO and other gases following the exposure. A delivery system as described herein is configured, according to some embodiments, as being capable of scavenging gNO.

A delivery system according to some of the present embodiments can additionally or alternatively comprise one or more, preferably two, vacuum devices for scavenging gaseous nitric oxide and other gases that may form during the exposing to gNO. The vacuum devices can be placed or held above, or distal to, the tumor, or to the delivering device, during administration, for example, about 15 cm away. The vacuum devices can vacuum all the gases from the area at a rate of at least about 50 liter per minute. The vacuuming of the gas can be done in a pulsed or continuous manner, preferably synchronized with gNO administration. Purging the gNO delivery system, for example, with nitrogen, before and/or after can also be performed. Purging can also be performed intermittently during the procedure.

According to some embodiments, a chemical hood is placed above the tumor sample and used to apply vacuum and scavenge gNO. The hood can be placed about 15 cm above the tumor. The hood can vacuum gas at a flow rate of at least about 50 LPM.

The vacuum system can exchange the gas at a flow rate of at least 50 liter per minute.

In each of the embodiments that relate to a vacuum application, a NO and NOx filtering pump can be placed in the discharge line. A soda lime filter, such as a Sofnolime filter, or a similar filter that can absorb NOx molecule, can be used. In each instance, vacuuming can be done in a pulsed or continuous manner, preferably synchronized with gNO administration or not.

According to some of any of the embodiments described herein, the delivery system is configured for delivering gNO to one or more administration sites by a positive pressure gradient, and scavenging gNO from the one or more administration sites by a negative pressure gradient.

According to some of any of the embodiments described herein, the delivery system comprises a gas supply passage in fluid communication with gas supply openings. The delivery system can further comprise an exhaust passage in fluid communication with an exhaust opening. The delivery system can comprise one or more gas supply openings and/or one or more exhaust openings, and/or one or more gas supply passages and/or one or more exhaust passages.

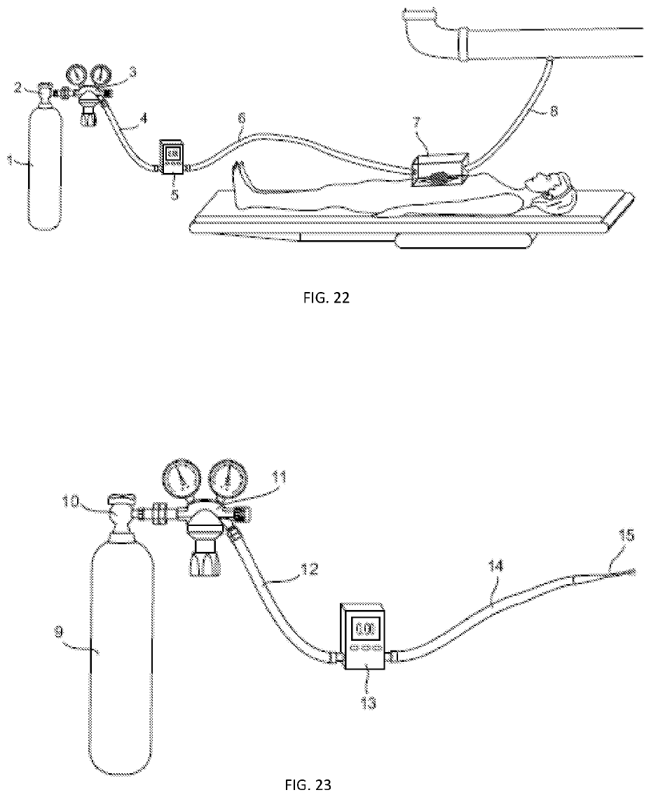

An exemplary system for exposure to gNO and scavenging of gaseous nitric oxide comprises a container or box, as illustrated, for example in FIG. 22 and described in further detail hereinunder, that can be filled with high dose (e.g., concentration) gNO at a volume of at least 0.5 Liter Per Minute (LPM) supplied from a tank (a gas reservoir). The container can have 2 or more holes, or ports. A first hole or port can be an input hole, sized to allow insertion of at least a portion of the tumor or of a bodily organ containing the tumor. An output hole can be connected to a discharge conduit or pipe that can remove or evacuate excessive NOx gases out from the box to the outside air, avoiding or minimizing the risk of contaminating the room and overexposing staff and the treated subjects (patients). Applying a vacuum can be in a pulsed or continuous manner, preferably synchronized with gNO administration.

An exemplary delivery system can comprise a small bore inner cannula that delivers an adequate dose of gNO to the target site, for example, a target site of from about 1 mm² to about 2 cm2 in size. The delivery system can further comprise an outer lumen through which a vacuum may be applied to scavenge excess gNO away from tumor cells.

In an exemplary method, a tumor sample is inserted into a hole that substantially matches the sample diameters. An additional output pore in the box enables lowering the pressure. The excessive gas is cleared up from the box through this output pore as described above. For example, the gas can flow into the box containing the tumor for 2 seconds followed by a suction of the gas for 2 seconds.

An exemplary delivery system comprises an outer lumen or cannula, trocar, tube, etc. An inner lumen or cannula, tube, etc. can be disposed coaxially inside of the outer lumen. The inner lumen is disposed approximately centrally in the outer lumen, although other configurations are also contemplated.

In some embodiments of such an exemplary delivery system, a space, preferably an exhaust space, is between the outer lumen and inner lumen. The exhaust space can be annular or may take any other configuration and/or geometry. A tip can be attached to the inner lumen at the distal end, and is in fluid communication with the inner lumen. In some embodiments, the tip comprises a wire mesh or screen that accesses the space inside of the inner lumen. The delivery device may be advanced to an administration site in the retracted configuration. In some embodiments, the tip is rounded and seals the outer lumen when it is in the retracted position.

When the tip of the system is brought adjacent to an administration site, the system is adjusted to its extended configuration in order to affect the administration of the gNO. In the extended configuration, an exhaust path is opened between the distal end of the outer lumen and the tip. gNO is delivered through the inner lumen. The gNO exits the inner lumen at the tip that is in fluid communication with the inner lumen. A wire mesh or screen at the distal end of the tip may assist in diffusing the gNO gas as it exists the device. The exhausted gNO gas returns to the device at the exhaust path. In embodiments, a vacuum is applied to the exhaust space between the outer lumen and the inner lumen in order to attract the exhausted gNO. The exhausted gNO is then brought through the exhaust space to exit and be disposed of appropriately. In this way, the device is capable of scavenging gNO from an administration site.

In an alternative configuration, the flow of gNO could be reversed such that gNO is delivered through the space between the outer lumen and inner lumen and removed from the administration site through the inner lumen. In this alternative configuration, a vacuum can be applied to the inner lumen and a positive pressure of gNO is applied to the space defined by the inner and outer lumens. In another alternative, the tip is permanently secured to the outer lumen as well as the inner lumen, such that the tip does not have retracted and extended configurations. In this alternative, permanent passages are provided in the outer lumen for gNO to be expelled from the device or sucked into the device by vacuum. For example, the permanent passages could be small holes or slits radially disposed around the outer lumen, preferably near the distal end of the outer lumen so as to be near the tip of the device.

According to some of any of the embodiments described herein, the delivery system comprises a double-needle system in which a suction needle is located adjacent or proximal to the gas delivery needle (e.g., a distance of between 3 mm and 1 cm). The suction needle can decrease or maintain intra-tumoral pressure. Further, two or more needles can be spaced at least about 2 mm apart. gNO can be delivered at a flow rate of at least about 0.01 LPM as described herein. The needles can be designed to have holes along the length of the needle. In example, the diameter of the holes is about 1 mm and disposed every 2 mm. The needles can be disposed within a lumen, placed outside of the tumor mass, while the shaft of the needle can be placed inside the tissue. The length of the needles can be selected to be at least half the tumor's longest dimension. Vacuuming gas through one or more suction needles or holes can be done in a pulsed or continuous manner, preferably synchronized with gNO administration. For example, the gNO can flow into the tumor for 2 seconds followed by applying a vacuum or suction for 2 seconds. One or more, such as a plurality, of needles or an array of needles, such as nano-sized or micron-sized needles can be used. In some embodiments, the gas is injected into the tumor by means of an array of needles with a spacing of about 0.5 cm. The ratio of suction needles and delivery needles can be 1:10 to 10:1, preferably 1:1. The suction needles can be designed to remove less than about 1 liter per minute per cm gas or fluid.

According to some of the any of the embodiments described herein, gNO is applied to a tumor sample through one or more intra-tumoral channels. For example, a channel 2 mm in width respective to every 4 mm of tissue can be formed through which gas can be delivered, for example at a flow rate higher than 0.01 LPM, directly to the tumor mass by a needle that is placed at the center of each channel, while the gas is cleared up from a scavenging channel. The flow rate in the scavenging channel can be lower than the delivery channel, such as at least 0.001 LPM less than the gNO delivery flow rate. The vacuuming can be accomplished through suction needles or hoses and can be pulsed or continuous, preferably synchronized with gNO administration. For example, the gNO can flow into the tumor for 2 seconds followed by a suction of the gas for 2 seconds.

According to some of the any of the embodiments described herein, gNO is sprayed onto a tumor sample.

According to some embodiments of a delivery system, the delivery device is or comprises a capping configuration, and a gas tubing is connected to the capping configuration, as described in further detail hereinunder. For example, the capping configuration can have two or more layers as described herein. The gNO can be connected to a first layer and the NOx suction can be connected to a second layer.

The following describes exemplary configurations of a delivery system and of methods employing same that can be used with some embodiments. Additional exemplary configurations are described in the Examples section that follows.

Referring in this regard to the figures, FIG. 22 is a schematic illustration of a delivery system in which gaseous NO is stored in a cylinder 1 with a valve 2 such as, but not limited to, a one-way valve, on top of it, which is connected to a pressure regulator 3.

Cylinder 1 is optionally and preferably disposable. This is particularly advantageous when the gas is toxic, as in the case of gNO, so that the disposable cylinder can be connected to the delivery system immediately before treatment, and disposed immediately after treatment, thus reducing the time at which the toxic substance is in the treating or operating room. In various exemplary embodiments of the invention the volume of cylinder 1 is sufficiently small so that the amount of gas in cylinder 1 is no more than the typical gas dose to be delivered to the tissue. This is particularly advantageous when the gas is toxic, as in the case of gNO, because in the event of undesired leakage of the gas into the treating room, the total amount of gas that can be leaked is small, compared to the size of the room, thus reducing the risk of inhaling a hazardous concentration of the gas by the subject or medical personnel.

A digital flow controller 5 is connected to the pressure regulator 3 by a designated gas tubing 4. gNO is delivered to an exposure box 7 by a gas tubing 6 that is connected to the box 7 at its distal end and to flow controller 5 at its proximal end. The tumor is placed inside box 7 before gNO delivery. gNO is then delivered to box 7 while excessive gas is evacuated by suctioning through a designated gas tubing 8 to an evacuation system that is preferably connected to the medical center pipe to allow releasing of the gas outside (not shown).

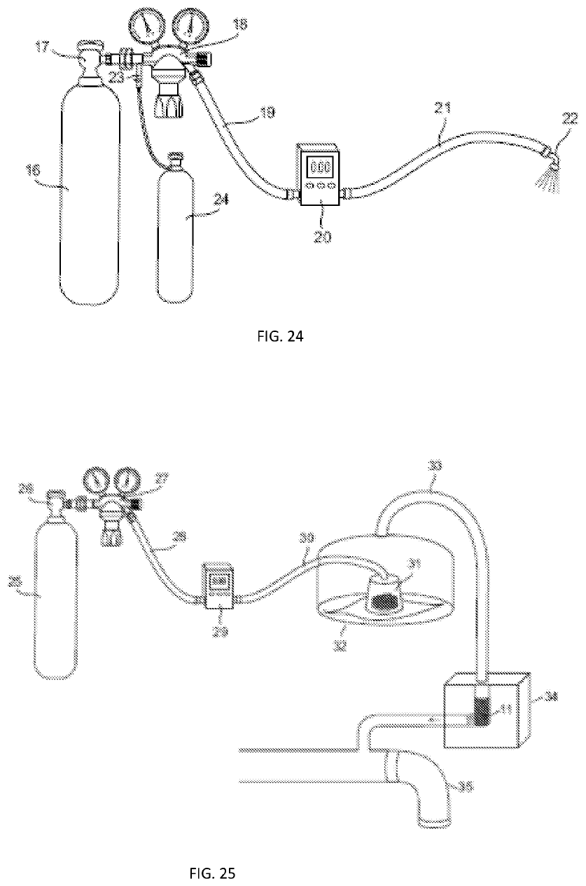

The suctioning of the gas can be done in a pulsed or continuous manner, preferably synchronized with gNO administration. Purging of the gNO delivery system; including the pressure regulator 3, the digital flow controller 5, the exposure box 7 and the tubing lines 4, 6, and 8 can be performed before and/or after treatment. Nitrogen can be used as an exemplary purge gas for purging the system. The purge gas can be introduced from a separate cylinder, as further detailed hereinbelow in the description accompanying FIG. 24.

FIG. 23 presents a schematic illustration of a delivery system in which gNO is delivered into the tumor by a needle 15. Components 9-14 are same as 1-6 as shown in FIG. 22, respectively.

FIG. 24 presents a schematic illustration of a delivery system in which gNO is delivered onto a tumor's surface by spraying. Components 16-21 are the same as components 1-6, respectively, in FIG. 22. The delivery system illustrated in FIG. 24 comprises a delivery device 22 that configured for spraying the gNO to a tumor's surface. The delivery system can comprise a second cylinder 24 designated for purging the accumulated gas in the pressure regulator 18, and optionally and preferably one or more other components of the system, such as, but not limited to, the designated gas tubing(s) 19 and 21, and/or the flow controller 20. The release of the accumulated gas is done automatically, electronically or manually by a valve 23.

Second cylinder 24 may purge the accumulated gas by introducing into the designated gas tubing(s) of the system (e.g., tubing 19 and optionally tubing 21) a purge gas, optionally and preferably an inert purge gas, such as, but not limited to, nitrogen. The purge operation may begin by operating valve 23 to release the purge gas from the second cylinder 24 into the designated gas tubing(s) and prevent release of gNO from first cylinder 16 into the designated gas tubing(s). The purge gas washes the designated gas tubing(s), the pressure regulator 18, and the flow controller 20, from gNO remnants and other substances (e.g., oxygen) that can react with the gNO.

Preferably, the purging is for a predetermined time period, e.g., at least 1 minute. In some embodiments of the present invention the purging includes multiple pressurize and depressurize cycles, followed by continuous flow of the purge gas. The Inventors found that such a protocol speeds up the purge and ensures that gas remnants and other substances are more effectively washed out, even from dead ended gas pathways and corners. In some embodiments of the present invention the depressurizing parts of the cycles includes application of vacuum to the gas flow lines. This can be done, for example, by temporarily connecting one of the ports of the valve 23 to a vacuum source (not shown). Alternatively, the valve 23 can include an additional port to which the vacuum source is connected, and the depressurizing parts of the cycles can include switching the valve to a state in which fluid communication is established between the additional port and the tubing 19.

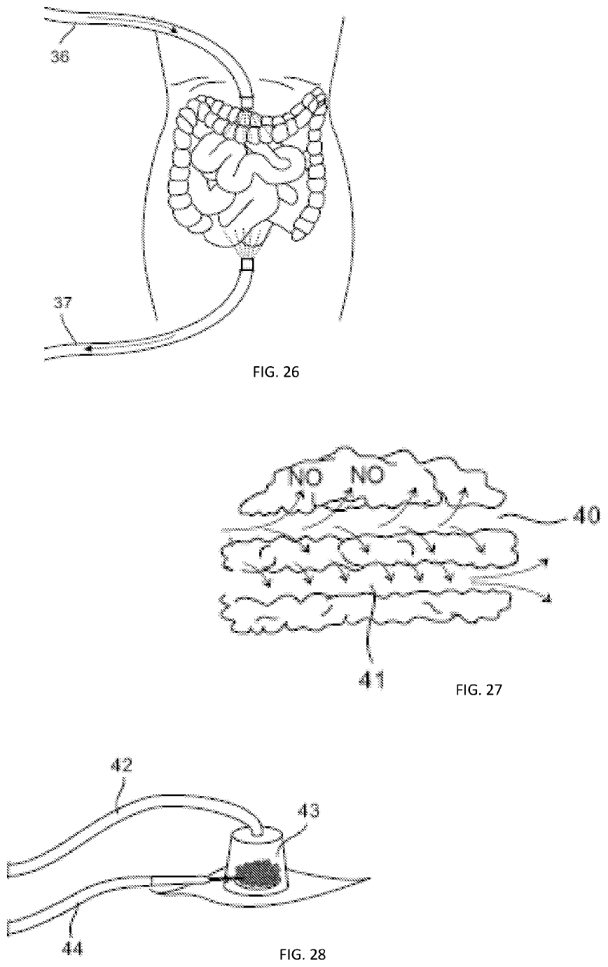

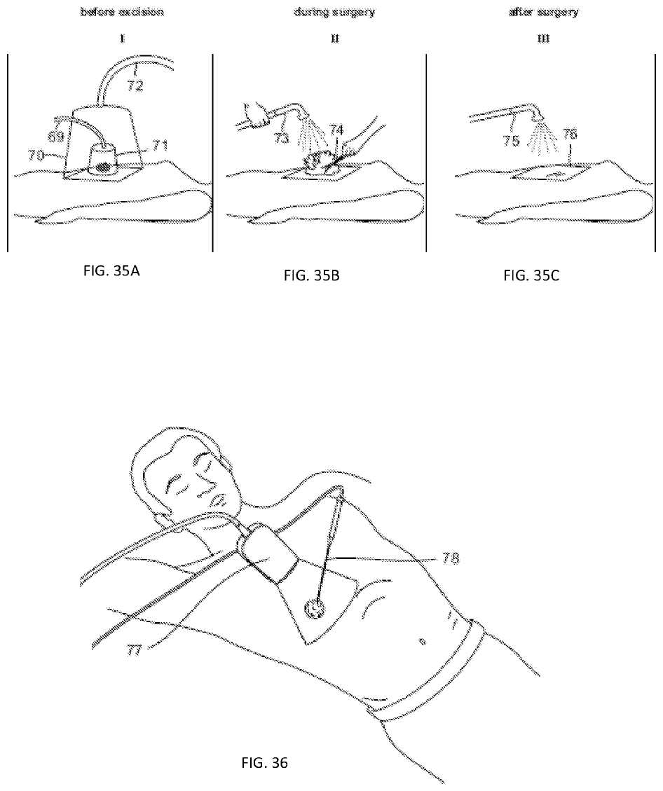

While FIG. 24 illustrates cylinder 24 as smaller in size than cylinder 16, this need not necessarily be the case, since, for some applications, it may be desired to make a cylinder substantially smaller in size than cylinder 24, particularly when cylinder 16 is sufficient small so that the amount of gas in the cylinder is not more than the typical gas dose to be delivered to the tissue, as further detailed hereinabove. Further, while the second cylinder 24 is only illustrated in FIG. 24, it is to be understood, that preferred embodiments of the present invention, such or similar second cylinder 24 can be incorporated in any of the delivery systems described herein, such as, but not limited to, the systems and configurations of the embodiments illustrated in FIGS. 22, 23, 25, 26, 27, 28, 29, 30, 31, 32, 33, 34, 35A-C, 36, and 38.

FIG. 25 presents a schematic illustration of a delivery system in which gNO is delivered by means of a cap 31. Components 25-30 are same as components 1-6 shown in FIG. 22. When such a system is operated, gNO is delivered directly to the tumor's outer layer by cap 31 that covers the tumor and is placed inside a sealed cap 32 that is connected to a gas tubing 33 through which excessive gas is evacuated by suctioning and is optionally and preferably also filtered by a filter 34 (e.g., a tube filled with soda lime, available under the tradename Sofnolime), filtering out gNO and optionally also NO, from the gas mixture coming out of the tumor cap 31 to release clean air.

Typically, the clean air is released into the pipes 35 of the medical center.

FIG. 27 presents a schematic illustration of an exemplary configuration in which a gas delivery channel 40 is placed inside the tumor. To control the intra-tumoral pressure, excessive gas is evacuated through an intra-tumoral gas-scavenging channel 41 that can be connected to a NOx filtering pump such as described herein.

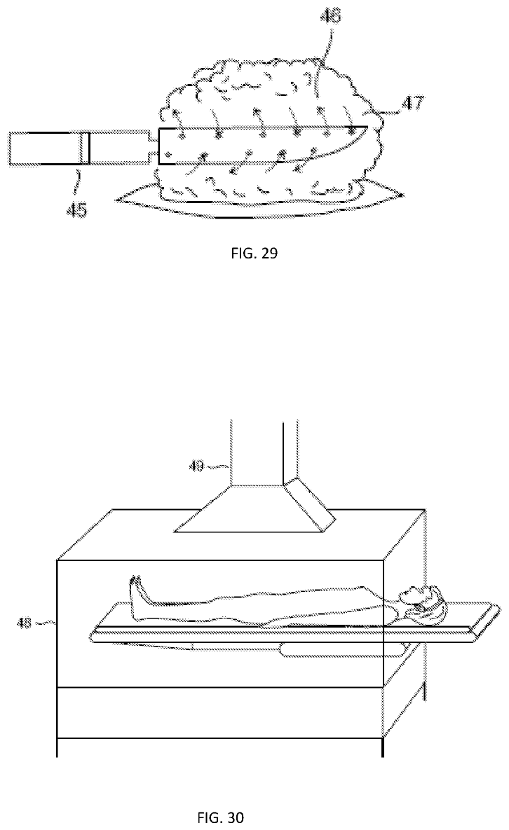

FIG. 28 presents a schematic illustration of a configuration that combines two of the aforementioned delivery methodologies, which can increase (e.g., maximize) the number of gNO exposed cancer cells during treatment. gNO is delivered from a cylinder (such as shown in FIG. 22, 23 or 24) to a gas tubing 42 that is connected to a cap 43 that covers the entire tumor mass; while the tumor cells are at the same time treated by means of a needle 44 as a delivery device. FIG. 29 presents a schematic illustration of a configuration in which gNO treatment is combined with a surgical procedure, in which a tumor is excised while using a gNO-releasing scalpel. The scalpel components include a holder 45, gNO releasing pores 46 that are in fluid communication with a gNO reservoir (not shown), and gas evacuation pores 47 that are connected to NO and NOx filtering pump as described herein (not shown). The vacuuming of the gas can be done in a pulsed or continuous manner.

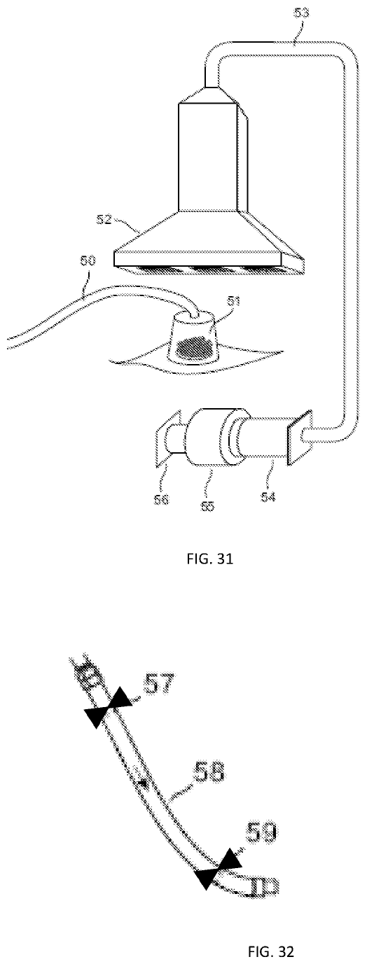

FIG. 31 presents a schematic illustration of a configuration in which gNO is delivered to the tumor via a tubing 50 connected to a capping 51 which covers tumor cells. To enable evacuation of NO and NOx from the tumor surrounding, a chemical hood 52 is placed directly above capping 51, and is connected to a gas tubing 53, which in turn is connected to a pump (not shown) via an NO and NOx filtering system 54-56.

FIG. 32 presents a schematic illustration of a one-way valve 57 that is placed nearby the main valve of the gNO cylinder (not shown) and is in fluid communication with a gas tubing 58, which in turn is in fluid communication with an additional one-way valve 59 located at the end of tubing 58 and nearby the delivery device (not shown).

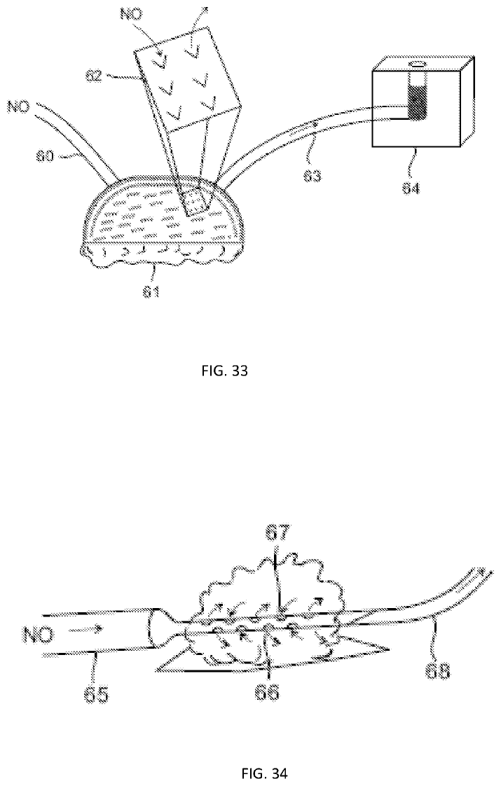

FIG. 33 presents a schematic illustration of a configuration in which gNO is supplied to a gas tubing 60 that is connected to patch 61 having a plurality of needles 62 (shown in an enlarged form) on its surface. Needles 62 include gNO input needles and gas output needles for a rapid clear up of excessive gas. The output needles are connected to a suction tubing 63 that is connected to a pump (not shown) via an NO and NOx filter 64.

FIG. 34 presents a schematic illustration of an exemplary configuration in which gNO is delivered through a device 65 that includes a perforated spray intra-tumoral needle with input pores 67 and output pores 66. NO and NOx clear-up is performed via a gas tubing 68 which is connected to the lumen of device 65 and to a pump via an NO and NOx filter (not shown).

Uses: According to specific embodiments, there is provided a vaccine obtainable according to the methods.

Such a vaccine can be used for example for inducing an immunological response to a tumor in a subject or preventing and/or treating a tumor in a subject in need.

Thus, the methods of some embodiments of the present invention further comprise administering a therapeutically effective amount of the tumor vaccine to a subject in need thereof.

Further, according to an aspect of the present invention, there is provided a method of preventing and/or treating a tumor in a subject in need thereof, the method comprising administering a therapeutically effective amount of the tumor vaccine, thereby preventing and/or treating the tumor in the subject.

According to an additional or an alternative aspect of the present invention, there is provided the tumor vaccine for use in preventing and/or treating a tumor in a subject in need thereof.

According to an additional or an alternative aspect of the present invention, there is provided a method of inducing an immunological response to a tumor in a subject in need thereof, the method comprising administering a therapeutically effective amount of the tumor vaccine, thereby inducing the immunological response to the tumor in the subject.

According to an additional or an alternative aspect of the present invention, there is provided a method of preventing and/or treating a tumor in a subject in need thereof, the method comprising administering to the subject a therapeutically effective amount of a tumor vaccine, wherein said tumor vaccine is obtainable by ex-vivo exposing a tumor sample to gaseous nitric oxide (gNO), and wherein said administering is effected at least twice, thereby preventing and/or treating the tumor in the subject.

According to an additional or an alternative aspect of the present invention, there is provided a tumor vaccine for use in preventing and/or treating a tumor in a subject in need thereof, wherein said tumor vaccine is obtainable by ex-vivo exposing a tumor sample to gaseous nitric oxide (gNO), and wherein the tumor vaccine is administered to the subject at least twice.

According to an additional or an alternative aspect of the present invention, there is provided a method of inducing an immunological response to a tumor in a subject in need thereof, the method comprising administering to the subject a therapeutically effective amount of a tumor vaccine, wherein said tumor vaccine is obtainable by ex-vivo exposing a tumor sample to gaseous nitric oxide (gNO), and wherein said administering is effected at least twice, thereby inducing the immunological response to the tumor in the subject.

As used herein throughout, the term “tumor” describes a plurality of cells or a tissue composed of the plurality of cells that are characterized by abnormal cell growth and which serve no physiological function.

By “abnormal cell growth” it is meant uncontrolled, progressive proliferation of the cells, which is no longer under normal bodily control. The growth of a tumor tissue typically exceeds, and is uncoordinated with, that of the normal cells or tissues around it.

“Abnormal cell growth” also describes cell growth that is independent of normal regulatory mechanisms (e.g., loss of contact inhibition), including, for example, abnormal growth of: (1) cancerous (or cancer) cells that proliferate by expressing a mutated tyrosine kinase or over-expression of a receptor tyrosine kinase; (2) benign and malignant cells of other proliferative diseases in which aberrant tyrosine kinase activation occurs; (3) any tumors that proliferate by receptor tyrosine kinases; (4) any tumors that proliferate by aberrant serine/threonine kinase activation; and (5) benign and malignant cells of other proliferative diseases in which aberrant serine/threonine kinase activation occurs.

The term “tumor” is also referred to herein and in the art as “neoplastic tissue” encompasses benign, pro-malignant and malignant tumors.

The phrase “cell growth”, as used herein, for example in the context of “tumor cell growth”, unless otherwise indicated, is used as commonly used in oncology, where the term is principally associated with growth in cell numbers, which occurs by means of cell reproduction (i.e., proliferation) when the rate of the latter is greater than the rate of cell death (e.g., by apoptosis or necrosis), to produce an increase in the size of a population of cells, although a small component of that growth may in certain circumstances be due also to an increase in cell size or cytoplasmic volume of individual cells.

An agent that inhibits cell growth can thus do so by either inhibiting proliferation or stimulating cell death, or both, such that the equilibrium between these two opposing processes is altered.

The term “tissue” describes an ensemble of cells, not necessarily identical, but from the same origin, that together carry out a specific function.

According to specific embodiments, the term tissue does not include dissociated cells.

According to specific embodiment, the tissue comprises a plurality of cell types.

According to additional embodiments, the tissue comprises vasculature.

The phrase “inhibiting cell growth” describes, as indicated above, altering the equilibrium between cells proliferation and cell death such that a rate of cell death is increased and is higher than the proliferation rate, resulting in a reduced or nullified number of viable cells. Thus, this phrase encompasses reducing or inhibiting proliferation of cells, killing cells, and/or reducing a volume of a tissue formed of the cells (a tumor tissue).

The phrase “tumor growth”, as used herein, unless otherwise indicated, is principally associated with an increased mass or volume of the tumor, primarily as a result of tumor cell growth.

A tumor as described herein can be a primary tumor or a secondary tumor.

The term “malignant tumor” describes a tumor that is not self-limited in its growth, is capable of invading into adjacent tissues, and may be capable of spreading to distant tissues (metastasizing). The term “benign tumor” describes a tumor which is not malignant (i.e. does not grow in an unlimited, aggressive manner, does not invade surrounding tissues, and does not metastasize).

The term “primary tumor” describes a tumor that is at the original site where it first arose.

The term “secondary tumor” describes a tumor that has spread from its original (primary) site of growth to another site, close to or distant from the primary site, and is also referred to herein and in the art as metastasis, or as metastasizing tumor. The term “secondary tumor” as used herein also describes recurrent tumor, which can be at the original site as the primary tumor and/or at another site, as a metastasizing tumor.

According to some of any of the embodiments described herein, the tumor is a malignant tumor, for example, a malignant cancerous tumor, and the tumor cells are cancer or cancerous cells.

According to these embodiments, the methods and uses as described herein in any of the respective embodiments are for treating and/or controlling cancer or a cancerous tumor is a subject in need thereof.

The methods and uses as described herein are for treating a subject having a primary cancer tumor, a metastasizing cancer and/or a recurrent cancer, as described herein.

The term “cancer” encompasses malignant and benign tumors as well as disease conditions evolving from primary or secondary tumors, as described herein.

Examples of benign tumors include, without limitation, lipomas, chondromas, adenomas, pilomatricomas, teratomas, and hamartomas.

Cancers treatable according to embodiments of the invention include, but are not limited to, carcinomas, sarcomas, blastomas, and germ cell tumors. Carcinomas include, without limitation, adenocarcinomas (e.g., small cell lung cancer, kidney, uterus, prostate, bladder, ovary and/or colon adenocarcinoma) and epithelial carcinomas.

Examples of cancers treatable according to embodiments of the invention include, without limitation, adenocarcinoma, adrenal tumors (e.g., hereditary adrenocortical carcinoma), biliary tract tumors, bladder cancer, bone cancer, brain cancer, breast cancer (e.g., ductal breast cancer, invasive intraductal breast cancer, sporadic breast cancer, susceptibility to breast cancer, type 4 breast cancer, breast cancer-1, breast cancer-3, and/or breast-ovarian cancer), bronchogenic large cell carcinoma, cervical cancer (e.g., cervical carcinoma), carcinosarcoma, choriocarcinoma, cystadenocarcinoma, dermatofibrosarcoma protuberans, ductal carcinoma, Ehrlich-Lettre ascites, embryonal rhabdomyosarcoma, endocrine neoplasia, endometrial cancer (e.g., endometrial carcinoma), ependimoblastoma, epidermoid carcinoma, epithelial adult tumor, epithelioma, erythroleukemia (e.g., Friend and/or lymphoblast), extraskeletal myxoid chondrosarcoma, fibrosarcoma, gallbladder carcinoma, ganglioblastoma, gastrointestinal tract tumors (e.g., colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6, hereditary nonpolyposis type 7, small and/or large bowel carcinoma, esophageal carcinoma, tylosis with esophageal cancer, stomach carcinoma, pancreatic carcinoma, and/or pancreatic endocrine tumors), germ cell tumor (male germ cell tumor, and/or testicular and/or ovarian dysgerminoma), giant cell tumor, glial tumor, glioma, glioblastoma (e.g., glioblastoma multiforme, astrocytoma), head & neck cancer, heterohybridoma, heteromyeloma, histiocytoma, hybridoma (e.g., B-cell), hypernephroma, insulinoma, islet tumor, keratoma, large cell carcinoma, leiomyoblastoma, leiomyosarcoma, leukemia (e.g., acute lymphatic leukemia, acute lymphoblastic leukemia, acute lymphoblastic pre-B cell leukemia, acute lymphoblastic T cell leukemia, acute megakaryoblastic leukemia, monocytic leukemia, acute myelogenous leukemia, acute myeloid leukemia, acute myeloid leukemia with eosinophilia, B-cell leukemia, basophilic leukemia, chronic myeloid leukemia, chronic B-cell leukemia, eosinophilic leukemia, Friend leukemia, granulocytic or myelocytic leukemia, hairy cellleukemia, lymphocytic leukemia, mast cell leukemia, megakaryoblastic leukemia, monocytic leukemia, monocytic-macrophage leukemia, myeloblastic leukemia, myeloid leukemia, myelomonocytic leukemia, plasma cell leukemia, pre-B cell leukemia, promyelocytic leukemia, subacute leukemia, T-cell leukemia, lymphoid neoplasm, predisposition to myeloid malignancy, and/or acute nonlymphocytic leukemia), Li-Fraumeni syndrome, liposarcoma, liver cancer (e.g., hepatoblastoma, hepatocellular carcinoma, hepatocellular cancer, and/or hepatoma), lung cancer (e.g., Lewis lung carcinoma, small cell carcinoma and/or non-small cell carcinoma) lymphoma (e.g., Hodgkin's disease, non-Hodgkin's lymphoma, B-cell lymphoma, diffuse large B-cell lymphoma (DLBCL), Burkitt lymphoma, cutaneous T-cell lymphoma, histiocytic lymphoma, lymphoblastic lymphoma, T-cell lymphoma, and/or thymic lymphoma), lymphosarcoma, lynch cancer family syndrome II, mammary tumor, mastocytoma, medulloblastoma, medullary carcinoma, melanoma, mesothelioma, metastatic tumor, monocyte tumor, mucoepidermoid carcinoma, multiple glomus tumors, multiple meningioma, myelodysplastic syndrome, myeloma (e.g., multiple myeloma), nasopharyngeal cancer, nephroblastoma, nervous tissue glial tumor, nervous tissue neuronal tumor, neurinoma, neuroblastoma, neurogenic tumor, non-melanoma skin cancer, oat cell carcinoma, oligodendroglioma, osteochondroma, osteomyeloma, ovarian cancer (e.g., epithelial ovarian cancer, ovarian carcinoma, serous ovarian cancer, and/or ovarian sex cord tumors), papillary carcinoma, papilloma, paraganglioma (e.g., familial nonchromaffin), pheochromocytoma, pituitary tumor (invasive), placental site trophoblastic tumor, plasmacytoma, prostate cancer (e.g., prostate adenocarcinoma), renal cancer (e.g., Wilms' tumor type 2 or type 1), retinoblastoma, rhabdoid tumors (e.g., rhabdoid predisposition syndrome), rhabdomyosarcoma, sacrococcygeal tumor, sarcoma (e.g., Ewing's sarcoma, histiocytic cell sarcoma, Jensen sarcoma, myxosarcoma, osteosarcoma, reticulum cell sarcoma, soft tissue sarcoma and/or synovial sarcoma), schwannoma, small cell carcinoma, spindle cell carcinoma, spinocellular carcinoma, squamous cell carcinoma (e.g., in head and neck), subcutaneous tumor, teratocarcinoma (e.g., pluripotent), teratoma (e.g., immature teratoma of ovary), testicular cancer (e.g. testicular germ cell tumor), transitional cell carcinoma, Turcot syndrome with glioblastoma, thymoma, thyroid cancer (e.g., follicular, medullary and/or papillary thyroid cancer), trichoepithelioma, trophoblastic tumor, undifferentiated carcinoma, uterine cancer, uterine cervix carcinoma.

Methods and uses of the present embodiments can be used to treat one or more solid tumors.

As used herein, the term “solid tumor” refers to those conditions, such as cancer, that form an abnormal tumor mass, such as sarcomas, carcinomas, and lymphomas. For example, solid tumors can include, but are not limited to, ovarian tumors, prostate tumors, skin tumors, lung tumors, breast tumors, liver tumors, brain tumors, CNS tumors, kidney tumors, colon tumors, bladder tumors, intestinal tumors, melanomas, gliomas, ependymomas, oligodendrogliomas, oligoastrocytomas, astrocytomas, glioblastomas, and medulloblastomas. Suitable examples of solid tumor diseases include, but are not limited to, non-small cell lung cancer (NSCLC), neuroendocrine tumors, thyomas, fibrous tumors, metastatic colorectal cancer (mCRC), and the like. In certain embodiments, the solid tumor disease is an adenocarcinoma, squamous cell carcinoma, large cell carcinoma, and the like.

According to some embodiments, the cancer is or comprises a solid tumor, and can be, for example, adenocarcinoma, adrenal tumors (e.g., hereditary adrenocortical carcinoma), biliary tract tumors, bladder cancer, bone cancer, brain cancer, breast cancer (e.g., ductal breast cancer, invasive intraductal breast cancer, sporadic breast cancer, susceptibility to breast cancer, type 4 breast cancer, breast cancer-1, breast cancer-3, and/or breast-ovarian cancer), bronchogenic large cell carcinoma, cervical cancer (e.g., cervical carcinoma), carcinosarcoma, choriocarcinoma, cystadenocarcinoma, dermatofibrosarcoma protuberans, ductal carcinoma, Ehrlich-Lettre ascites, embryonal rhabdomyosarcoma, endocrine neoplasia, endometrial cancer (e.g., endometrial carcinoma), ependimoblastoma, epidermoid carcinoma, epithelial adult tumor, epithelioma, extraskeletal myxoid chondrosarcoma, fibrosarcoma, gallbladder carcinoma, ganglioblastoma, gastrointestinal tract tumors (e.g., colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6, hereditary nonpolyposis type 7, small and/or large bowel carcinoma, esophageal carcinoma, tylosis with esophageal cancer, stomach carcinoma, pancreatic carcinoma, and/or pancreatic endocrine tumors), germ cell tumor (male germ cell tumor, and/or testicular and/or ovarian dysgerminoma), giant cell tumor, glial tumor, glioma, glioblastoma (e.g., glioblastoma multiforme, astrocytoma), head & neck cancer, heterohybridoma, heteromyeloma, histiocytoma, hybridoma (e.g., B-cell), hypernephroma, insulinoma, islet tumor, keratoma, large cell carcinoma, leiomyoblastoma, liposarcoma, liver cancer (e.g., hepatoblastoma, hepatocellular carcinoma, hepatocellular cancer, and/or hepatoma), lung cancer (e.g., Lewis lung carcinoma, small cell carcinoma and/or non-small cell carcinoma) lymphoma (e.g., Hodgkin's disease, non-Hodgkin's lymphoma, B-cell lymphoma, diffuse large B-cell lymphoma (DLBCL), Burkitt lymphoma, cutaneous T-cell lymphoma, histiocytic lymphoma, lymphoblastic lymphoma, T-cell lymphoma, and/or thymic lymphoma), lymphosarcoma, lynch cancer family syndrome II, mammary tumor, mastocytoma, medulloblastoma, medullary carcinoma, melanoma, mesothelioma, metastatic tumor, monocyte tumor, mucoepidermoid carcinoma, multiple glomus tumors, multiple meningioma, myelodysplastic syndrome, myeloma (e.g., multiple myeloma), nasopharyngeal cancer, nephroblastoma, nervous tissue glial tumor, nervous tissue neuronal tumor, neurinoma, neuroblastoma, neurogenic tumor, non-melanoma skin cancer, oat cell carcinoma, oligodendroglioma, osteochondroma, osteomyeloma, ovarian cancer (e.g., epithelial ovarian cancer, ovarian carcinoma, serous ovarian cancer, and/or ovarian sex cord tumors), papillary carcinoma, papilloma, paraganglioma (e.g., familial nonchromaffin), pheochromocytoma, pituitary tumor (invasive), placental site trophoblastic tumor, plasmacytoma, prostate cancer (e.g., prostate adenocarcinoma), renal cancer (e.g., Wilms' tumor type 2 or type 1), retinoblastoma, rhabdoid tumors (e.g., rhabdoid predisposition syndrome), rhabdomyosarcoma, sacrococcygeal tumor, sarcoma (e.g., Ewing's sarcoma, histiocytic cell sarcoma, Jensen sarcoma, myxosarcoma, osteosarcoma, reticulum cell sarcoma, soft tissue sarcoma and/or synovial sarcoma), schwannoma, small cell carcinoma, spindle cell carcinoma, spinocellular carcinoma, squamous cell carcinoma (e.g., in head and neck), subcutaneous tumor, teratocarcinoma (e.g., pluripotent), teratoma (e.g., immature teratoma of ovary), testicular cancer (e.g. testicular germ cell tumor), transitional cell carcinoma, Turcot syndrome with glioblastoma, thymoma, thyroid cancer (e.g., follicular, medullary and/or papillary thyroid cancer), trichoepithelioma, trophoblastic tumor, undifferentiated carcinoma, uterine cancer, uterine cervix carcinoma.

One of skill in the art will appreciate that the methods and uses provided herein for inhibiting abnormal growth of tumor cells or tissue, and are for treating, controlling, or preventing cancer may be generally applicable to all known or to-be-discovered cancerous cell phenotypes and cancerous growths.

The present embodiments relate to any size and shape of tumors, including large, spread and amorphic cancerous outgrowths.

The methods and uses of some embodiments provided herein may be especially useful for the treatment, control, and/or prevention of tumors (e.g., cancerous tumors) at localized sites, including inoperable tumors, tumors where localized treatment would be beneficial, and solid tumors.

According to some of any of the embodiments described herein, the methods and uses of the present embodiments are for inhibiting growth of cells of a primary tumor.

According to some of any of the embodiments described herein, the methods and uses of the present embodiments are for inhibiting growth of cells of a secondary tumor.

In some embodiments, a method or a use as described herein is for boosting the immune system of a subject against cancer.

The methods and uses as described in these embodiments can be for inhibiting growth and/or killing cells or tissue of a primary tumor in the subject, e.g., by stimulating an anti-tumor immunological response in the subject against its own tumor.

Alternatively or in addition, the methods and uses as described in these embodiments can be used for preventing and/or inhibiting growth of a secondary (recurrent and/or metastasizing) tumor in the subject, e.g., by stimulating an anti-tumor immunological response in the subject against its own tumor.

Alternatively, or in addition, the methods as uses as described in these embodiments can be used for vaccinating the subject against the tumor.

Vaccine preparation: Specific embodiments of the claimed invention disclose an ex-vivo method of producing a tumor vaccine from a tumor sample.

The produced vaccine can be administered to the subject with or without an adjuvant.

According to embodiments of this aspect of the embodiments of the present invention, there is provided a method for induction of a universal cancer-specific vaccination for a cancer patient. In some embodiments, one or more tumor samples treated ex-vivo as described herein in any of the respective embodiments and any combination thereof, and libraries of treated tumor cells or a processed preparation thereof are built and categorized according to the primary cancer type. The one or more tumor samples can be from the same patient. The one or more tumor samples can be from the two or more patients.

Thus, according to specific embodiments, a bank of tumor samples or processed preparation thereof from multiple donors can be established.

According to other specific embodiments, there is provided a method of producing a patient-specific tumor vaccine, such that the tumor sample is obtained from the same subject.

According to specific embodiments, the tumor sample comprises a primary tumor.

According to specific embodiments, the tumor sample comprises a secondary tumor. According to specific embodiments, the tumor sample comprises a combination of a primary and a secondary tumor.

According to specific embodiments, the tumor sample is autologous to the subject.

According to specific embodiments, the tumor sample is allogeneic to the subject.

According to specific embodiments, the tumor sample is of the same type of the tumor to be treated and/or prevented.

According to specific embodiments, the tumor sample is a tissue sample.

According to specific embodiments, a volume of the tumor tissue sample is 1-1000 mm³.

According to specific embodiments, a volume of the tumor tissue sample is 1-500 mm³, 1-300 mm³ or 1-200 mm³.

According to other specific embodiments, the tumor sample is a single tumor cells or clumps sample.

According to specific embodiments, the tumor cells in suspension are single tumor cells or clumps.

According to specific embodiments, the clumps do not exceed 100 cells per clump.

According to specific embodiments, the tumor sample is a dissociated cells sample.

The term “dissociated cells” refers to single cells and or cell clumps not exceeding 100 cells per clump which result from mechanical or chemical dissociation and/or enzymatic digestion of a tissue.

According to specific embodiments, the method can comprise obtaining the tumor sample from a subject. Thus, according to some of any of the embodiments described herein for these aspects, prior to the ex-vivo treatments disclosed herein, a sample of the tumor, which can be a whole tumor or a portion thereof, is obtained. This can be done, for example, by surgery, biopsy, or any other method known in the art for tumor excision, resection, or sampling. Following, according to specific embodiments, single cells or clumps are obtained from the tumor sample by methods well-known in the art, as further described hereinabove and below. According to specific embodiments, tumor cells are dissociated from a tissue sample.

Following the treatment (e.g. culturing, exposing, subjecting to a preservation protocol, suspending and/or titrating), the tumor sample can further be processed to obtain a tissue fragment, single cells, clumps and/or an extracted biomaterial, collectively referred to herein as “preparation thereof”.

According to specific embodiments, the processing is effected following the exposing.

According to specific embodiments, the processing is effected following the culturing.

According to specific embodiments, the processing is effected following the subjecting to a preservation protocol.

According to specific embodiments, the processing is effected following the pH titration.

According to specific embodiments, the preparation thereof comprises a fragment of the tumor sample.

According to specific embodiments, the fragment is 1-300 mm³, 1-200 mm³, 1-150 mm³ or 1-100 mm³.

According to specific embodiments the preparation thereof comprises dissociated cells.

According to specific embodiments, the preparation thereof comprises single cells or clumps.

According to specific embodiments, the preparation thereof comprises an extracted biomaterial which can be e.g. a proteinaceous biomaterial and/or genetic biomaterial. An exemplary processing procedure is described in the Examples section that follows. According to specific embodiments, the extracted biomaterial is not viable.

In some of these embodiments, the tumor sample or preparation thereof is administered to the subject as a tumor vaccine per se.

According to specific embodiments, the tumor cells of the vaccine are non-proliferative and/or non-viable.

Thus, according to specific embodiments, the method further comprising determining viability and/or proliferation of the tumor vaccine.

Methods of determining viability and proliferation are well known in the art and non-limiting examples are further described in the Examples section which follows.

According to specific embodiments, the tumor sample or the preparation thereof is administered to the subject by means of antigen presenting immune cells.

Hence, according to specific embodiments, the method comprising contacting antigen presenting immune cells with said tumor sample or preparation thereof to thereby obtain immune cells presenting an antigenic biomaterial of said tumor sample.

Antigen presenting immune cells can be obtained from the subject, for example, from a blood organ of a subject by methods well known in the art, e.g., by drawing a blood sample from the subject, or from a sample of a lymphatic blood organ.

According to some of any of the embodiments described herein, the blood organ sample is processed to isolate therefrom immune cells (e.g., dendritic cells). This can be done by methods known in the art. An exemplary method is described in the Examples section that follows.

According to some of any of the embodiments, subsequent to isolating the immune cells from the sample drawn from the subject, the method further comprises proliferating the immune cells. Proliferating the immune cells can be performed by culturing the immune cells is a culturing medium under conditions that allow or promote proliferation, using techniques known in the art.

The immune cells are then contacted (e.g., incubated) with the treated tumor sample or preparation thereof under suitable conditions to obtain antigen presenting immune cells containing the antigenic biomaterial obtained from the tumor sample.

According to specific embodiments, the antigen presenting cells are autologous to the subject.

According to specific embodiments, the antigen presenting cells are non-autologous and can be allogeneic or non-allogeneic or modified, as described herein, to thereby provide antigen presenting immune cells that present allogeneic antigenic biomaterial. In some embodiments, the antigen presenting immune cells are engineered (modified) to make them suitable for immunotherapy purposes, namely, non-allogenic, as described hereinabove. The immune cells can be engineered to be non-allogenic (non-alloreactive) by any suitable method known in the art.

In exemplary embodiments, prior to administering the antigen presenting immune cells from one patient to another patient, the immune cells are modified to avoid or overcome an immune response to the administered cells by the receiving patient. In some embodiments, the antigen presenting immune cells are modified to knockout or reduce human leukocyte antigen (HLA), for example HLA-A, HLA-B, HLA-C, or HLA receptor expression. The modified antigen presenting immune cells derived from donors can evade an immune response and provide a foundation whereby cells from a single donor can be administered to multiple recipients. Such modified antigen presenting immune cells are also contemplated according to the present embodiments.

These immune cells can then be used as immune response stimulator, immunization, vaccination, or simple as medicament, when administered to a patient, which can be the patient from which the immune cells were derived, or any other patient, preferably upon being processed to prevent rejection, as described herein.

The thus obtained immune cells are then and administered to the subject, using methods known in the art.

An exemplary procedure for executing a method as described herein is provided in the Examples section that follows.

According to another aspect of some embodiments of the present invention, there are provided antigen presenting immune cells comprising the antigenic biomaterial described herein.

The immune cells can be as described herein in any of the respective embodiments.

According to another aspect of some embodiments of the present invention, there is provided a method of stimulating an anti-tumor immune response in a subject in need thereof, which comprises administering to the subject the antigen presenting immune cells containing the antigenic biomaterial as described herein.

According to another aspect of some embodiments of the present invention, there is provided a use of the antigen presenting immune cells containing the antigenic biomaterial as described herein for stimulating an anti-tumor immune response in a subject in need thereof.

According to embodiments of these methods and uses, the antigen presenting immune cells are obtained by incubating immune cells (e.g., antigen-presenting immune cells) with the antigenic biomaterial described herein. The immune cells can be derived from the subject to be treated or from another subject, including the subject from which the tumor sample was obtained.

According to specific embodiments, the tumor sample, the preparation thereof or the vaccine can be subjected to a preservation protocol during the method of producing the vaccine, e.g. following isolation of the tumor sample, prior to culturing, following culturing, following exposure to gNO, following titration, following processing of the tumor sample, following preparation of the antigen presenting cells presenting the tumor antigenic materials etc.

The preservation ability is important for clinical and research applications. Preservation of cells and preparation thereof permits their transportation between locations, as well as completion of safety and quality control testing. Preservation also permits the development of a ‘manufacturing paradigm’ for therapies, thereby maximizing the number of products that can be produced.

According to specific embodiments, the preservation protocol comprises freezing e.g. by cryopreservation. Cryopreservation agents which can be used include, but are not limited to, dimethyl sulfoxide (DMSO) (Lovelock and Bishop, 1959, Nature 183: 1394-1395; Ashwood-Smith, 1961, Nature 190: 1204-1205), glycerol, polyvinylpyrrolidone (Rinfret, 1960, nn. N.Y. Acad. Sci. 85: 576), polyethylene glycol (Sloviter and Ravdin, 1962, Nature 196: 548), albumin, dextran, sucrose, ethylene glycol, i-erythritol, D-ribitol, D-mannitol (Rowe et al., 1962, Fed. Proc. 21: 157), D-sorbitol, i-inositol, D-lactose, choline chloride (Bender et al., 1960, J. Appl. Physiol. 11520), amino acids (Phan The Tran and Bender, 1960, Exp. Cell Res. 20: 651), methanol, acetamide, glycerol monoacetate (Lovelock, 1954, Biochem. J. 56: 265), and inorganic salts (Phan The Tran and Bender, 1960, Proc. Soc. Exp. Biol. Med. 104: 388; Phan The Tran and Bender, 1961, in Radiobiology, Proceedings of the Third Australian Conference on Radiobiology, Ilbery, P. L. T., ed., Butterworth, London, p. 59).

A controlled slow cooling rate is desirable in some instances. Different cryoprotective agents (Rapatz et al., 1968, Cryobiology 5(1): 18-25) and different cell types have different optimal cooling rates (see, e.g., Rowe and Rinfret, 1962, Blood 20: 636; Rowe, 1966, Cryobiology 3(1): 12-18; Lewis et al., 1967, Transfusion 7(1):17-32; and Mazur, 1970, Science 168939-949 for effects of cooling velocity on survival of marrow-stem cells and on their transplantation potential).

The heat of fusion phase where water turns to ice should be minimal. The cooling procedure can be carried out by use of, e.g., a programmable freezing device or a methanol bath procedure.

Programmable freezing apparatuses allow determination of optimal cooling rates and facilitate standard reproducible cooling. Programmable controlled-rate freezers such as Cryomed or Planar permit tuning of the freezing regimen to the desired cooling rate curve.

After thorough freezing, cells can be rapidly transferred to a long-term cryogenic storage vessel. According to specific embodiments, samples can be cryogenically stored in liquid nitrogen (−196° C.) or its vapor (−165° C.). Such storage is greatly facilitated by the availability of highly efficient liquid nitrogen refrigerators, which can be containers with an extremely low vacuum and internal super insulation, such that heat leakage and nitrogen losses are kept to an absolute minimum.

Other methods of cryopreservation, or modifications thereof, are available and envisioned for use (e.g., cold metal-mirror techniques; Livesey and Linner, 1987, Nature 327: 255; Linner et al., 1986, J. Histochem. Cytochem. 34(9): 1123-1135; see also U.S. Pat. No. 4,199,022 by Senken et al., U.S. Pat. No. 3,753,357 by Schwartz, U.S. Pat. No. 4,559,298 by Fahy.

Frozen cells are preferably thawed quickly (e.g., in a water bath maintained at 37°−41° C.) and chilled immediately upon thawing.

It may be desirable to treat the cells in order to prevent cellular clumping upon thawing. To prevent clumping, various procedures can be used, including but not limited to the addition before and/or after freezing of Dnase (Spitzer et al., 1980, Cancer 45: 3075-3085), low molecular weight dextran and citrate, hydroxyethyl starch (Stiff et al., 1983, Cryobiology 20: 17-24), etc.

The cryoprotective agent, if toxic in humans, should be removed prior to therapeutic use. One way in which to remove the cryoprotective agent is by dilution to an insignificant concentration.

Combination therapy: According to specific embodiments, the tumor vaccines and methods described herein can be combined with an additional therapy for treating the disease associated with the tumor (e.g., cancer).

According to specific embodiments, the methods described herein can be effected in combination with an anti-cancer therapy.

Hence, according to an aspect of the present invention, there is provided an article of manufacture comprising as active ingredients the tumor vaccine and the anti-cancer therapy.

According to specific embodiments, the tumor vaccine and the anti-cancer therapy are provided in a co-formulation.

According to other specific embodiments, the tumor vaccine and the anti-cancer therapy are provided in separate formulations.

Suitable anti-cancer therapy includes, for example, chemotherapy, radiotherapy, phototherapy and/or photodynamic therapy, surgery, nutritional therapy, ablative therapy, combined radiotherapy and chemotherapy, brachiotherapy, proton beam therapy, immunotherapy, cellular therapy and photon beam radiosurgical therapy, and any combination of the foregoing.

Chemotherapeutic drugs (e.g., anti-cancer drugs) that may optionally be co-administered to the subject prior to, concomitant with and/or subsequent to the tumor vaccine as described herein in any of the respective embodiments include, but are not limited to acivicin, aclarubicin, acodazole, acronine, adozelesin, aldesleukin, altretamine, ambomycin, ametantrone, aminoglutethimide, amsacrine, anastrozole, anthramycin, asparaginase, asperlin, azacitidine, azetepa, azotomycin, batimastat, benzodepa, bicalutamide, bisantrene, bisnafide, bizelesin, bleomycin, brequinar, bropirimine, busulfan, cactinomycin, calusterone, caracemide, carbetimer, carboplatin, carmustine, carubicin, carzelesin, cedefingol, chlorambucil, cirolemycin, cisplatin, cladribine, crisnatol, cyclophosphamide, cytarabine, dacarbazine, dactinomycin, daunorubicin, decitabine, dexormaplatin, dezaguanine, diaziquone, docetaxel, doxorubicin, droloxifene, dromostanolone, duazomycin, edatrexate, eflornithine, elsamitrucin, enloplatin, enpromate, epipropidine, epirubicin, erbulozole, esorubicin, estramustine, etanidazole, etoposide, etoprine, fadrozole, fazarabine, fenretinide, floxuridine, fludarabine, fluorouracil, flurocitabine, fosquidone, fostriecin, gemcitabine, hydroxyurea, idarubicin, ifosfamide, ilmofosine, interferon alfa-2a, interferon alfa-2b, interferon alfa-n1, interferon alfa-n3, interferon beta-Ia, interferon gamma-Ib, iproplatin, irinotecan, lanreotide, letrozole, leuprolide, liarozole, lometrexol, lomustine, losoxantrone, masoprocol, maytansine, mechlorethamine, megestrol, melengestrol, melphalan, menogaril, mercaptopurine, methotrexate, metoprine, meturedepa, mitindomide, mitocarcin, mitocromin, mitogillin, mitomalcin, mitomycin, mitosper, mitotane, mitoxantrone, mycophenolic acid, nocodazole, nogalamycin, ormaplatin, oxisuran, paclitaxel, pegaspargase, peliomycin, pentamustine, peplomycin, perfosfamide, pipobroman, piposulfan, piroxantrone, plicamycin, plomestane, porfimer, porfiromycin, prednimustine, procarbazine, puromycin, pyrazofurin, riboprine, rogletimide, safingol, semustine, simtrazene, sparfosate, sparsomycin, spirogermanium, spiromustine, spiroplatin, streptonigrin, streptozocin, sulofenur, talisomycin, tecogalan, tegafur, teloxantrone, temoporfin, teniposide, teroxirone, testolactone, thiamiprine, thioguanine, thiotepa, tiazofurin, tirapazamine, topotecan, toremifene, trestolone, triciribine, trimetrexate, triptorelin, tubulozole, uracil mustard, uredepa, vapreotide, verteporfin, vinblastine, vincristine, vindesine, vinepidine, vinglycinate, vinleurosine, vinorelbine, vinrosidine, vinzolidine, vorozole, zeniplatin, zinostatin, zorubicin, and any pharmaceutically acceptable salts thereof.

In some embodiments, the anti-cancer therapy comprises immunotherapy, including, for example, checkpoint inhibitors, CAR-T cell therapy, and/or vaccine adjuvants (e.g., interferon or saponin), and immune-adjuvants, such as aluminum salts, organic adjuvants, and genomic material-based adjuvants, such as CpG. Anti-tumor immunity can be further augmented by inhibition of immune suppressor cells.

According to some embodiments, the anti-cancer therapy comprises administration of an anti-cancer immune modulator agent.

As used herein, the term “anti-cancer immune modulator agent” refers to an agent capable of eliciting an immune response (e.g. T cell, NK cell) against a cancerous cell.

Exemplary such agents include a cancer antigen, a cancer vaccine, an anti-cancer antibody, a cytokine capable of inducing activation and/or proliferation of a T cell and an immune-check point regulator.

Alternatively or additionally, such modulators may be immune stimulators such as immune-check point regulators which are of specific value in the treatment of cancer.

As used herein the term “immune-check point regulator” refers to a molecule that modulates the activity of one or more immune-check point proteins in an agonistic or antagonistic manner resulting in activation of an immune cell.

As used herein the term “immune-check point protein” refers to a protein that regulates an immune cell activation or function. Immune check-point proteins can be either co-stimulatory proteins (i.e. transmitting a stimulatory signal resulting in activation of an immune cell) or inhibitory proteins (i.e. transmitting an inhibitory signal resulting in suppressing activity of an immune cell). According to some embodiments, the immune check-point protein regulates activation or function of a T cell. Numerous checkpoint proteins are known in the art and include, but not limited to, PD1, PDL-1, B7H2, B7H4, CTLA-4, CD80, CD86, LAG-3, TIM-3, KIR, IDO, CD19, OX40, 4-1BB (CD137), CD27, CD70, CD40, GITR, CD28 and ICOS (CD278).

According to some embodiments, the anti-cancer therapy comprises a surgical procedure, for example, resection or excision of at least a portion of the tumor.

Mode of administration to a subject: According to specific embodiments, the tumor vaccine is administered to the subject by injection (i.e. a solution or a suspension) or implantation.

According to specific embodiments, when the tumor vaccine comprises a tissue, the tissue is implanted in the subject.

The tumor vaccine disclosed herein, may be administered to a subject per se, or in a pharmaceutical composition where they are mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism Herein the term “active ingredient” refers to the vaccine disclosed herein (cells, tissues or preparations thereof), accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Pharmaceutical compositions of some embodiments of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the present invention thus may be formulated in conventional manner using one or more physiologically acceptable carriers comprising excipients and auxiliaries, which facilitate processing of the active ingredients into preparations, which can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer.

According to specific embodiments, the pharmaceutical composition is administered in a local rather than systemic manner, for example, via injection of the pharmaceutical composition directly into a tissue region of a patient.

According to specific embodiments, the tumor vaccine is administered intra-tumorally.

According to specific embodiments, the tumor vaccine is administered into the arm.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

According to specific embodiments, the administering is effected once.

According to specific embodiments, the administering is effected at least 2, at least 3, at least 4 or more times.

According to specific embodiments, the administering is effected at least twice.

A time interval between the administrations may be determined by the skilled person such as physicians, in accordance with the subject's response to treatment.

According to specific embodiments, a time interval between the at least two administrations is at least one week.

According to specific embodiments, a time interval between the at least two administrations is at least 2 weeks, at least 3 weeks, at least 4 weeks, at least 2 months, at least 3 months, at least 4 months or at least 5 months.

According to specific embodiments, a time interval between the at least two administrations is at least six months.

According to specific embodiments, administering is performed upon occurrence of a metastatic event and/or recurrence of the tumor.

The description herein of problems and disadvantages of known apparatus, methods, and devices is not intended to limit the invention to the exclusion of these known entities. Indeed, embodiments of the invention may include one or more of the known apparatus, methods, and devices without suffering from the disadvantages and problems noted herein.

As used herein throughout, “a” or “an” may mean one or more than one of an item.

As used herein throughout, the term “subject” and “patient” are used interchangeably herein and refer to both human and nonhuman animals at any gender and or any age. The term “nonhuman animals” of the disclosure includes all vertebrates, e.g., mammals and non-mammals, such as nonhuman primates, sheep, dog, cat, horse, cow, chickens, amphibians, reptiles, and the like, for medical and/or laboratory research purposes.

According to specific embodiments, the subject is a human subject.

According to specific embodiments, the subject suffer from or diagnosed with the pathology (i.e. tumor).

According to specific embodiments, the subject has been diagnosed with a primary tumor.

According to specific embodiments, the subject has been diagnosed with a secondary tumor.

According to other specific embodiments, the subject does not suffer or not diagnosed with the pathology (i.e. tumor).

According to specific embodiments, the subject is at risk to develop a tumor.

The term throughout “about” as used herein when referring to a measurable value such as an amount of weight, time, dose, etc. is meant to encompass variations of +/−20% or +/−10% from the specified amount, as such variations are appropriate to perform the disclosed method. In embodiments, the term “about” is meant to encompass variations of +/−5%. In embodiments, the term “about” is meant to encompass variations of +/−1%. In embodiments, the term “about” is meant to encompass variations of +/−0.1%.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition. As used herein, “treating” or “treatment” refers to any indicia of success in the treatment or amelioration of the disease or condition. Treating can include, for example, reducing or alleviating the severity of one or more symptoms of the disease or condition, or it can include reducing the frequency with which symptoms of a disease, defect, disorder, or adverse condition, and the like, are experienced by a patient. In embodiments, “treat” or “treating” means accomplishing one or more of the following: (a) reducing tumor size; (b) reducing tumor growth; (c) reducing or limiting development and/or spreading of metastases.

The phrase “a method of treating” or its equivalent, when applied to, for example, cancer, refers to a procedure or course of action that is designed to reduce or eliminate the number of cancer cells in an animal, or to alleviate the symptoms of a cancer. “A method of treating” cancer or another proliferative disorder does not necessarily mean that the cancer cells or other disorder will, in fact, be eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of an animal, is nevertheless deemed an overall beneficial course of action.

As used herein, “preventing” refers to the prevention of the disease or condition, e.g., tumor formation, in the patient. For example, if an individual at risk of developing a tumor or other form of cancer is treated with the methods of the present invention and does not later develop the tumor or other form of cancer, then the disease has been prevented in that individual. For example, “preventing” includes inhibiting the growth, spread, and development of cancerous cell phenotypes and growths. Methods and devices disclosed herein also may be useful for eradicating cancerous cell phenotypes and growths in animal, and preferably mammal, and more preferably human, bodies. As used herein, “eradicating” includes treating, controlling, suppressing, hindering, blocking, killing, and slowing the spread or development of cancerous cell phenotypes and growths.

Pharmaceutical compositions suitable for use in context of the present invention include compositions wherein the active ingredients are contained in an amount effective to achieve the intended purpose.

As used herein, a “therapeutically effective amount” is the amount of a composition sufficient to provide a beneficial effect to the individual to whom the composition is administered.

More specifically, a therapeutically effective amount means an amount of active ingredients effective to prevent and/or treat a tumor or induce an immunological response to a tumor.

Determination of a therapeutically effective amount is well within the capability of those skilled in the art, especially in light of the detailed disclosure provided herein.

For any preparation used in the methods of the invention, the therapeutically effective amount or dose can be estimated from animal models to achieve a desired concentration or titer.

Such information can be used to more accurately determine useful doses in humans.

Toxicity and therapeutic efficacy of the active ingredients described herein can be determined by standard pharmaceutical procedures in experimental animals. The data obtained from these animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide levels of the active ingredients which are sufficient to induce or suppress the biological effect (minimal effective concentration, MEC). The MEC will vary for each preparation, but can be estimated from in vitro data. Dosages necessary to achieve the MEC will depend on individual characteristics and route of administration.

The amount of a composition to be administered will, of course, be dependent on the subject being treated, the severity of the affliction, the manner of administration, the judgment of the prescribing physician, etc.

According to specific embodiments, the therapeutically effective amount comprises at least 1 mg, at least 10 mg, at least 50 mg or at least 100 mg of tumor sample or a fragment processed therefrom.

According to specific embodiments, the therapeutically effective amount comprises up to 1000 mg, up to 500 mg or up to 100 mg of tumor sample or a fragment processed therefrom.

According to specific embodiments, the therapeutically effective amount comprises 10-100 mg of said tumor sample or fragment processed therefrom.

According to specific embodiments, the therapeutically effective amount comprises at least 0.01×10⁶ cells, at least 0.1×10⁶ cells, at least 1×10⁶ cells, at least 10×10⁶ cells or at least 100×10⁶ cells.

According to specific embodiments, the therapeutically effective amount comprises up to 10×10⁷ cells, up to 1×10⁷ cells or up to 900×10⁶ cells.

According to specific embodiments, the therapeutically effective amount comprises 0.1×10⁶-900×10⁶ cells.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment.

Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, C A (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Experimental Methods for Examples 1-24

Study Drug: NO is a colorless, nonflammable, oxidizing gas. gNO was administered from 3.5-L cylinders, prepared, and supplied by Gordon Gas and Chemical (Tel Aviv, Israel) or 1 liter cylinders supplied by Impactor (Gan Yavne, Israel). As NO is a free radical that reacts within seconds, the inert gas nitrogen served as its stabilizing gas. NO concentrations used for this study were 0.015%-20% (150 to 200,000 ppm) NO in nitrogen. Air or nitrogen supplied from 3.5-liter cylinders served as the control gases.

All in vitro and ex vivo procedures were performed in a chemical hood in a well-ventilated room. Appropriate personal protection was used at all times. During gas exposure, the cylinder was placed inside a stabilizing apparatus. The gas was delivered via a pressure regulator through a designated silicone hose (International Biomedical, USA). The flow rate was set to 0.25-0.9 liters per minute (LPM) using a manual flow meter or to 0.002-0.05 LPM using a digital, remote-controlled mass flow controller (MFC, Bronkhorst).

Tumor celllines: The mouse colon carcinoma cell line, CT26WT, was cloned from CT26, an N-nitroso-N-methylurethane-induced, undifferentiated colon carcinoma cell line. The mouse mammary tumor cell line, 4T1, is a 6-thioguanine resistant cell line. When injected into BALB/c mice, 4T1 spontaneously produces highly metastatic tumors that can metastasize to the lung, liver, lymph nodes and brain while the primary tumor is growing in situ. The primary tumor does not have to be removed to induce metastatic growth. Panc 02.03 is a pancreatic adenocarcinoma epithelial cell line derived in 1995 from a primary tumor removed from the head-of-the-pancreas of a female with pancreatic adenocarcinoma. CT26, 4T1 and Panc 02.03 cell lines were grown in RPMI based media (ATCC 30-2001) supplemented with fetal bovine serum and pen-strep (both from Biological Industries). The LLC1/LU2 cell line was grown in DMEM based media (ATCC 30-2002) supplemented with fetal bovine serum and pen-strep (Biological industries). The B16.F10 cell lines was grown in DMEM based media (ATCC 30-2002) supplemented with fetal bovine serum and pen-strep (Biologicalindustries).

Preparation of tumor cells: Tumor cell suspensions in Hanks' Balanced Salt Solution (HBSS) (Biological Industries, Israel) or RPMI or DMEM based cell culture media, at appropriate concentration of 1-2×10⁵ cells/ml for in vitro studies or 2.5×10⁶-10.0×10⁶ cells/ml for in vivo studies were freshly prepared. Cells were grown to 70% confluency and were harvested using trypsin (BiologicalIndustries, Israel), and counted using a hemocytometer.

In vitro systems: One system involves a direct exposure of cancer cells to gNO in 96-well plate. Gaseous NO at 150 ppm-50,000 ppm was delivered at 0.3 LPM for 10 seconds—3 minutes. In another system, cancer cells were exposed to gNO in a box made of acrylic glass. Gaseous NO at 10,000-50,000 ppm was delivered at 0.9 LPM for 10 seconds—15 minutes.

The exposure of the cancer cells to gNO was done after removing the cell culture medium.

Immediately after gas exposure, cell culture medium was added, and the cells were incubated at a 37° C. and 5% CO₂ incubator over-night. To test cell viability, XTT assay and Annexin V-Propidium Iodide assays were performed, according to known protocols.

Mice: All in vivo experimental procedures were carried out in accordance with the protocol approved by the Ethics Committee on the Use and Care of Animals. Institutional Animal Care and Use Committee (IACUC): 67-09-2019 and IL-20-3-149. The in vivo assays were performed on Balb/c or C57BU6J mice: 7-10 weeks of age, obtained from Envigo (Ness-Ziona, Israel), unless otherwise indicated.

Inoculation of tumor cells: Cancer cell suspensions at a concentration of 2.5-10.0×10⁶ cells/ml were inoculated to the right flank of mice at a dose volume of 0.1 ml. Administration was performed as soon as possible following cell suspension preparation and after manual shaking prior to inoculation. The cell suspension was then aspirated into a 1-ml syringe with a 27G needle for subcutaneous (s.c.) injection.

Test article treatment: Mice were first anesthetized by an intraperitoneal (i.p) injection of 100 mg/kg ketamine and 20 mg/kg xylazine hydrochloride solution. After 10 minutes, tumor-bearing mice were treated with the indicated dose of gNO.

In all protocols, the gas was delivered from NO in N₂ cylinders. The outlet pressure was set to 2-3.5 bar using a pressure regulator that was connected directly to the cylinder.

In protocols that involve administration by needle injection, a silicone hose was connected directly to the pressure regulator on one end and to a manual or digital MFC on the other end. The flow was adjusted to 0.05 LPM-0.25 LPM. An additional silicone hose was connected to the flow controller as well as to a 23G hypodermic needle. The needle was inserted horizontally into the center of each tumor.

Tumor volume calculation: Local tumor growth was determined by measuring 3 mutually orthogonal tumor dimensions 2-3 times per week, according to the following formula:

$$Tumor volume = \frac{π}{6} \times [ Diameter 1 \times Diameter 2 \times Diameter 3 ]$$

or Tumor volume=Diameter 1× Diameter 2²/2, unless otherwise indicated. Diameter 1 represents length, Diameter 2 represents width and Diameter 3 represents height.

Challenge tumor inoculation: Up to 21 days after gas treatment was applied to the primary tumor, the appropriate cancer cell suspensions were prepared as described herein and inoculation of cells was repeated on the contralateral flank of the mice. Administration was performed as soon as possible following cell suspension preparation and after manual shaking prior to withdrawal of the cell suspension.

The percentage of challenge tumor take was monitored 2-3 times a week by taking a look at the inoculated site and touching this area to look for small tumors that are not yet visible. Naïve mice inoculated with tumor cells for the first time served as an internal control.

Winn assay: The Winn assay was performed by the s.c. injection of CT26 tumor cells 500,000 cells/mouse) mixed with either immune splenocytes (1,000,000 or 5,000,000 cells/mouse) or HBSS as a control.

Mixing of cells was performed immediately before injection to minimize leukocyte interactions within the syringe. Mice were observed 2-3 times a week and tumor development was determined by caliper measurements of tumor dimensions as described herein. Splenocytes were extracted from a CT26 tumor-bearing mouse, which was previously treated with 25,000 ppm NO for two 15-minute courses. This mouse was re-inoculated with CT26 cancer cells 12 days post treatment. Forty-four days after challenge assay conduction, no signs of tumor development were detected and this mouse was sacrificed and its splenocytes were extracted. The splenocytes were mixed with CT26 cells at a ratio of 1:2-1:10 (500,000 CT26 cells and 1,000,000 to 5,000,000 splenocytes, respectively). The cell mixtures were inoculated s.c. to the right flank of naïve Balb/c mice. Tumor take of visible outgrowths was monitored 2 to 3 times per week by taking a look at the inoculated site and touching this area to look for small tumors that are not yet visible Naïve mice inoculated s.c. with 500,000 CT26 served as the control group.

### Example 1

The effect of continuous gaseous nitric oxide (gNO) at 50,000 ppm compared to air (control) on the survival of CT-26 cancer cell line was tested.

CT-26 cells were prepared in RPMI medium and suspended in 96-well plates. The wells were exposed to the gas by a tubing that was directed at each well for 10, 60 or 180 seconds. The diameter of the gas tubing used was about 25% of the diameter of each well. Three wells were used for each gas and exposure time. The plates were thereafter placed in a 5% CO₂, 37° C. incubator for 24 hours and the viability of the cells was tested by XTT assay.

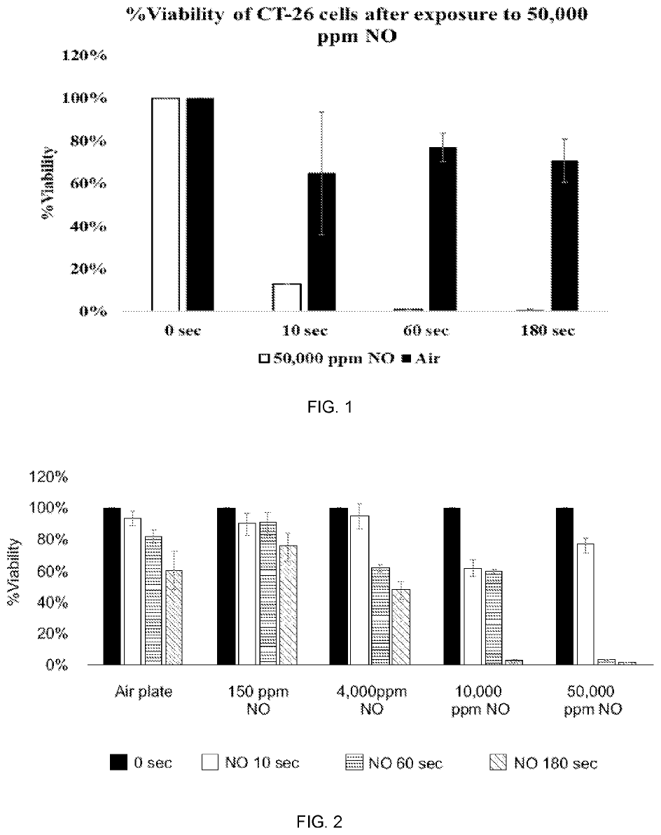

The obtained data is presented in FIG. 1 and shows about 1% viability of CT-26 cells after exposure to 50,000 ppm gNO for 10, 60, or 180 seconds, whereas more than 80% of the air-exposed CT-26 cells survived.

### Example 2

The effect of continuous gaseous nitric oxide (gNO) at 150 ppm, 4,000 ppm, 10,000 ppm, or 50,000 ppm, compared to air (control), on the survival of 4T1 cancer cell line was tested. 4T1 cells were prepared in RPMI medium and suspended in six 96-well plates. The wells were exposed to the gas by a tubing that was directed at each well for 10, 60 or 180 seconds. The diameter of the gas tubing was about 25% of the diameter of each well. Three wells were used for each gas and exposure time. The plates were thereafter placed in a 5% CO₂, 37° C. incubator for 24 hours and the viability of the cells was tested by XTT assay.

The obtained data is presented in FIG. 2 and show about 1-3% viability of 4T1 cells after exposure to 10,000 or 50,000 ppm gNO for 180 seconds, whereas more than 60% of the air-exposed 4T1 cells survived. In addition, less than 80% of 4T1 cells remained viable after 10 seconds when exposed to 10,000 ppm or 50,000 ppm gNO compared to more than 90% viability among air-exposed 4T1 cells. About 60% of 4T1 cells remain viable after 1 minute of exposure to 4,000-10,000 ppm gNO compared to more than 80% viability among air-exposed 4T1 cells.

### Example 3

The effect of continuous gaseous nitric oxide (gNO) treatment at 4,000 ppm, 10,000 ppm, or 50,000 ppm compared to air (control) on the survival of A549 cancer cellline was tested.

A549 cells were prepared in RPMI medium and suspended in four 96-well plates. The wells were exposed to the gas by a tubing that was directed at each well for 10, 60 or 180 seconds. The diameter of the gas tubing used was about 25% of the diameter of each well. Three wells were used for each gas and exposure time. The plates were placed in a 5% CO₂, 37° C. incubator for 15-18 hours and the viability of the cells was tested by XTT assay.

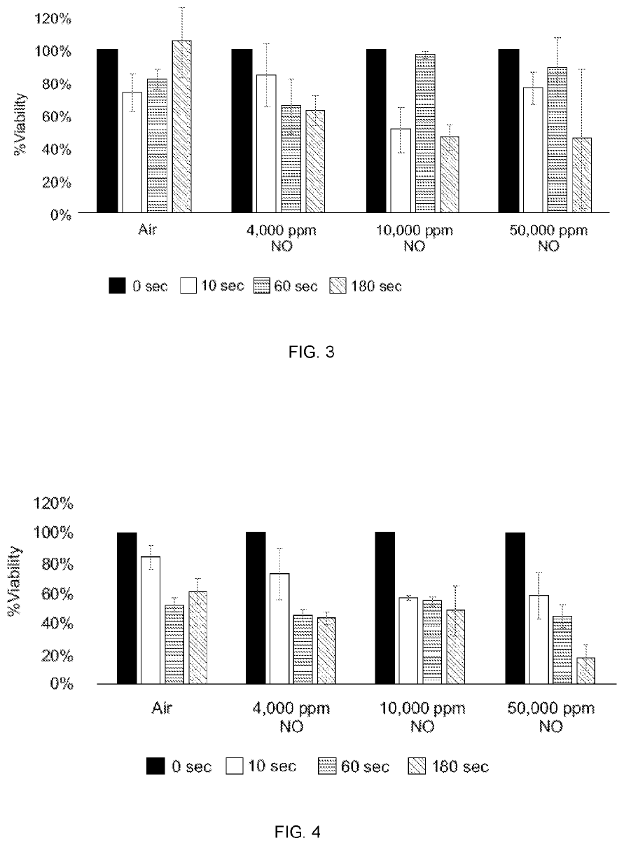

The obtained data is presented in FIG. 3 and shows about 45%-62% viability of A549 cells after exposure to 4000, 10,000, or 50,000 ppm gNO for 180 seconds, whereas about 100% of the air-exposed A549 cells survived. Results showed about 65%-96% viability of A549 cells after exposure to 4000, 10,000, or 50,000 ppm gNO for 60 seconds, whereas about 81% of the air-exposed A549 cells survived. Results showed about 50%-84% viability of A549 cells after exposure to 4000, 10,000, or 50,000 ppm gNO for 10 seconds, whereas about 73% of the air-exposed A549 cells survived.

### Example 4

The effect of continuous gaseous nitric oxide (gNO) at 4,000 ppm, 10,000 ppm, or 50,000 ppm compared to air (control) on the survival of PC-3 cancer cell line was tested.

PC-3 cells were prepared in RPMI medium and suspended in four 96-well plates. The wells were exposed to the gas by a tubing that was directed at each well for 10, 60 or 180 seconds. The diameter of the gas tubing used was about 25% of the diameter of each well. Three wells were used for each gas and exposure time. The plates were placed in a 5% CO₂, 37° C. incubator for 15-18 hours and the viability of the cells was tested by XTT assay.

The obtained data is presented in FIG. 4 and shows about 17% viability of PC-3 cells after exposure to 50,000 ppm gNO for 180 seconds and 43-48% viability after exposure to 4,000 ppm and 10,000 ppm gNO for 180 seconds. The viability of air-exposed PC-3 cells was 61% after 180 seconds. In addition, 56%-72% viability of PC-3 was observed after 10 seconds of exposure to 4,000 ppm, 10,000 ppm and 50,000 ppm gNO whereas 84% of air treated PC-3 cells survived after 10 seconds. After 1 minute of exposure to 4,000 ppm, 10,000 ppm and 50,000 ppm gNO the viability of PC-3 cells was 44%-55% compared to 52% viability of air-treated PC-3 cells.

### Example 5

The effect of continuous gaseous nitric oxide (gNO) at 10,000 ppm, 15,000 ppm, 20,000 ppm, 25,000 ppm, compared to air (control), on the survival of B16.F10 cancer cell line was tested.

B16.F10 cells were prepared in DMEM medium and suspended in 96-well plates. The plates were placed inside an approx. 2 liter exposure chamber. The plates were exposed to the gas by filling the chamber with gNO for 10 seconds, or 1, 3, 9 or 15 minutes. After exposure, the plates were placed in a 5% CO₂, 37° C. incubator for an overnight incubation and the viability of the cells was tested by XTT assay.

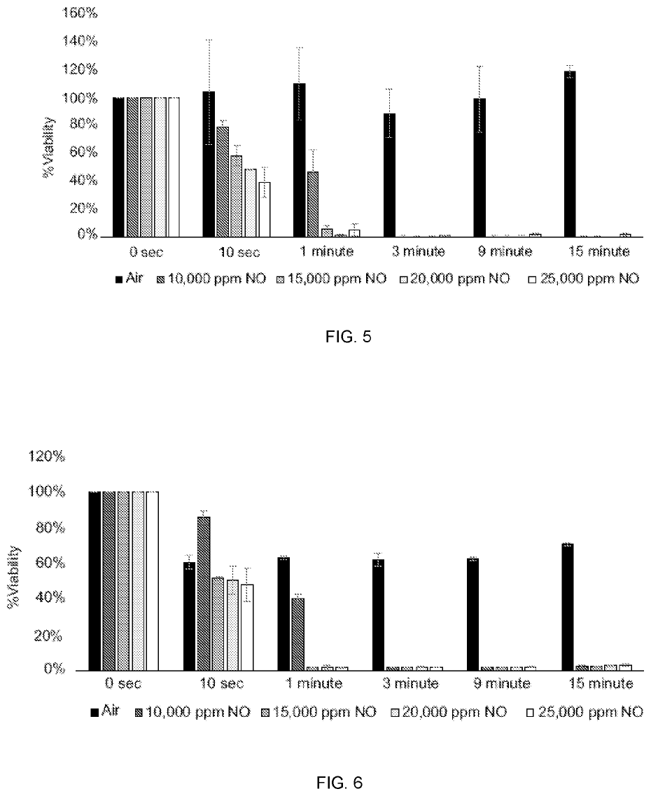

The obtained data is presented in FIG. 5, and shows less than 5% viability of B16.F10 cells after exposure to 10,000 ppm-25,000 ppm gNO for 3-15 minutes.

### Example 6

The effect of continuous gaseous nitric oxide (gNO) at 10,000 ppm, 15,000 ppm, 20,000 ppm, or 25,000 ppm, compared to air (control), on the survival of Panc02.03 cancer cell line was tested.

Panc02.03 cells were prepared in RPMI medium and suspended in 96-well plates. The plates were placed inside an approx. 2 liter exposure chamber. The plates were exposed to the gas by filling the chamber with gNO for 10 seconds, or 1, 3, 9 or 15 minutes. After exposure, the plates were placed in a 5% CO₂, 37° C. incubator for an overnight incubation and the viability of the cells was tested by XTT assay.

The obtained data is presented in FIG. 6 and shows less than 5% viability of Panc02.03 cells after exposure to 10,000 ppm-25,000 ppm gNO for 1-15 minutes.

### Examples 7 and 8

The effect of continuous gaseous nitric oxide (gNO) at 10,000 ppm, 15,000 ppm, 20,000 ppm, or 25,000 ppm, compared to air (control), on the survival of LLC1 cancer cell line was tested. LLC1 cells were prepared in DMEM medium and suspended in 96-well plates. The plates were placed inside an approx. 2 liter exposure chamber. The plates were exposed to the gas by filling the chamber with gNO for 10 seconds, or 1, 3, 9 or 15 minutes. After exposure, the plates were placed in a 5% CO₂, 37° C. incubator for an overnight incubation and the viability of the cells was tested by XTT assay.

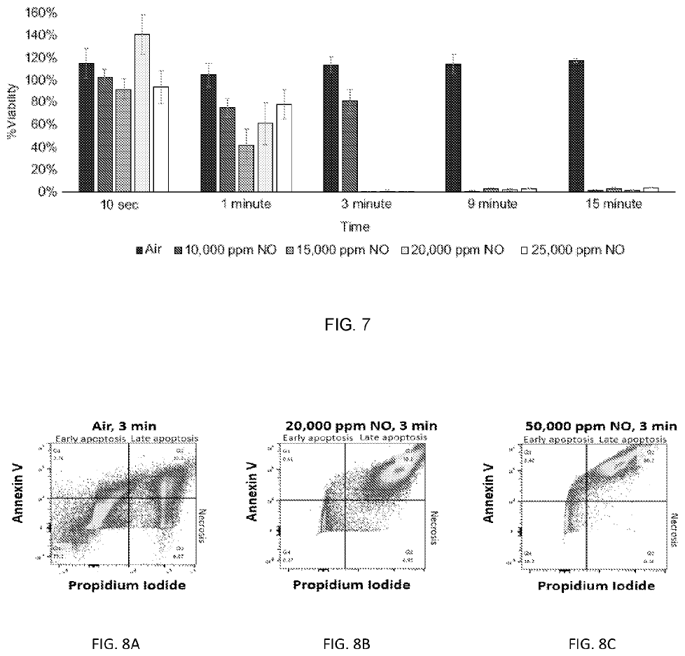

The obtained data is presented in FIG. 7 and shows less than 5% viability of LLC1 cells after exposure to 10,000 ppm-25,000 ppm gNO for 3-15 minutes.

In an additional set of experiments, the plates were exposed to the gas (at 20,000 ppm or 50,000 ppm) by filling the chamber with gNO for 3 minutes. After exposure, the plates were placed in a 5% CO₂, 37° C. incubator for an overnight incubation and the viability of the cells was tested via Annexin V—Propidium Iodide apoptosis-necrosis assay.

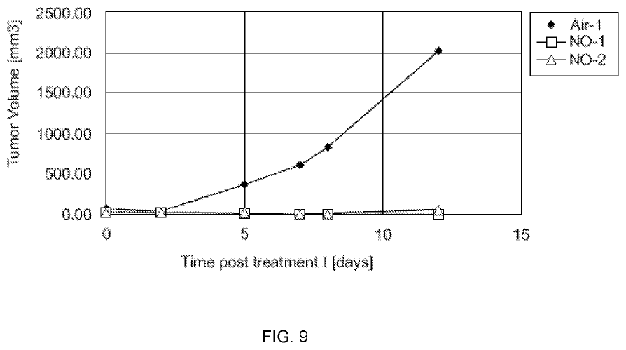

The obtained data is presented in FIGS. 8A-C and shows that 80%-90% of LLC1 cells are at late apoptosis after exposure to 20,000 ppm or 50,000 ppm gNO for 3 minutes, whereby nearly no apoptosis is observed for the air-exposed cells.

### Examples 9-11

The effect of local administration of 10,000-50,000 ppm gNO on CT26 tumor bearing mice was tested.

BALB/c female and male mice were obtained from Almog Diagnostic that purchased them from Envigo, Israel. The mice were 7-8 weeks of age. Animal care and experimentation were performed in accordance with the Bar-Ilan University guidelines.

CT-26, a colon carcinoma cell line was induced in a BALB/c mouse by chemical carcinogenesis.

Cells were cultured in Roswell Park Memorial Institute (RPMI) 1640 supplemented with 100 U/ml penicillin, 100 μg/ml streptomycin, 2 mM L-glutamine; 1 mM sodium pyruvate; 1% non-essential amino acid solution; 10% fetal calf serum. Cultures were maintained at 37° C. in a humidified incubator with a 5% CO₂ atmosphere.

Mice were inoculated subcutaneously with 5×10⁵ CT-26 cells suspended in 100 μL Hank's Balanced Salt Solution (HBSS) into the low lateral side of the back. Local tumor growth was determined by measuring two mutually orthogonal tumor diameters with a caliper. The volume of the tumor was calculated using the formula: Vol=D1×D2×D2/2, where D1, D2 are two mutually orthogonal tumor diameters.

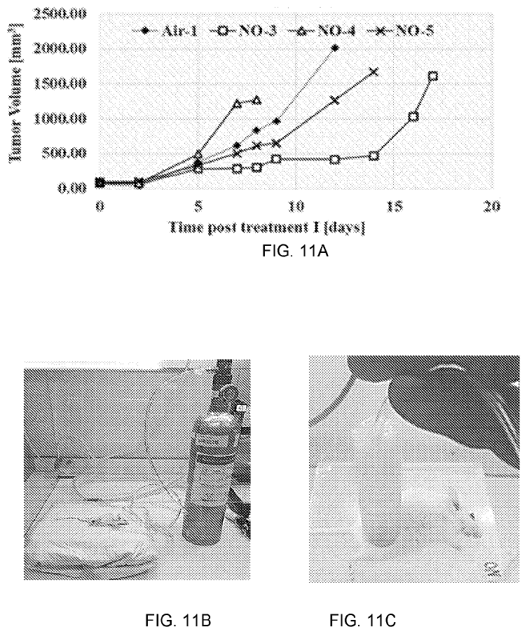

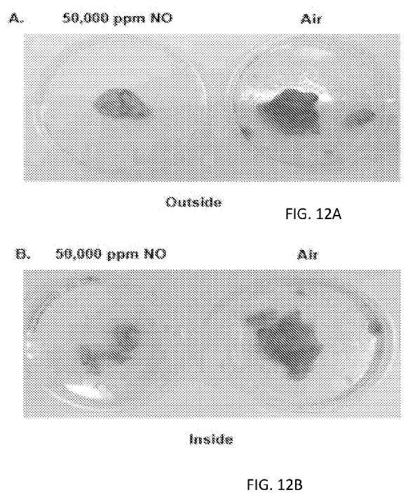

Air-1 is a female Balb/c mouse that was treated twice. On day 0 the tumor volume was 64.85 mm³. The treatments were: Day 0—Air was administered intra-tumoral by a needle for 2-3 minutes at a flow rate of 0.2 using a 3L cylinder; Day 8—gNO at 10,000 ppm was administered for 6 minutes intra-tumoral by a 23G needle, such as shown in FIG. 11B, at a flow rate of 0.3 LPM.

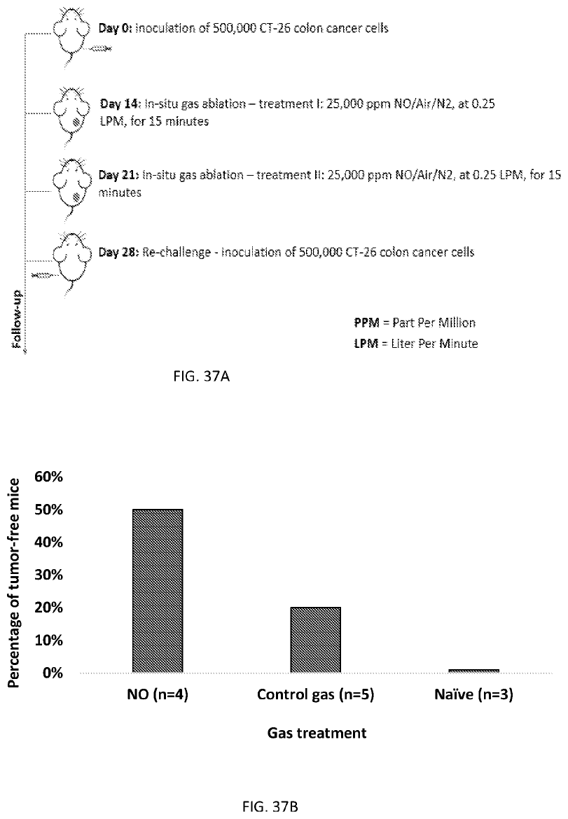

This was followed by air administration for 4 minutes administered to the outer layer of the tumor using a cap, such as shown in FIG. 11C. An exemplary system is also shown in FIGS. 23 and 36.

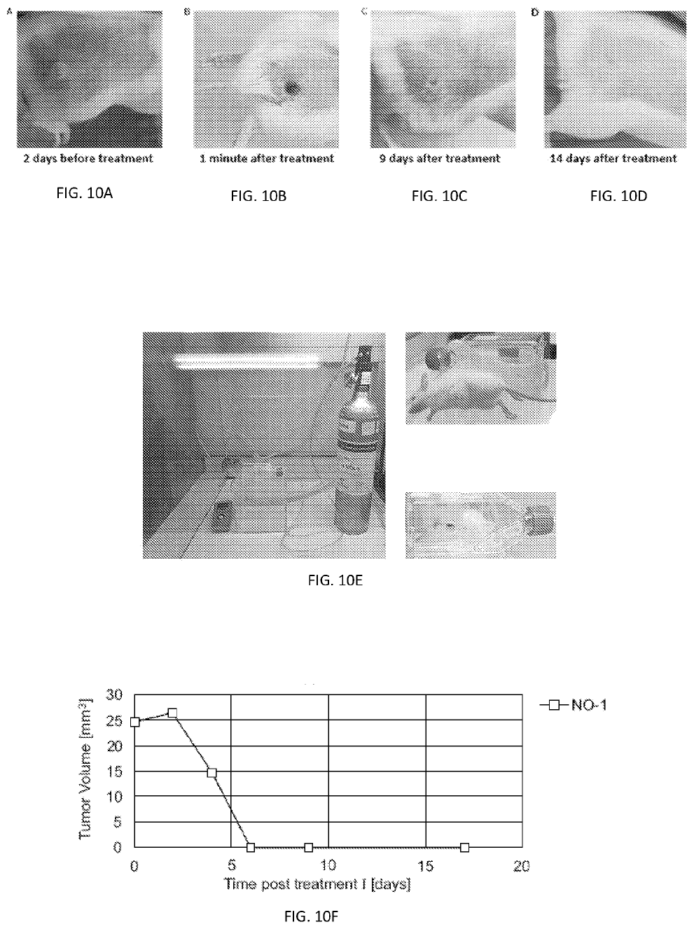

NO-1 is a female Balb/c mouse and was treated once. On day 0 the tumor volume was 24.79 mm³. gNO at 10,000 ppm at a flow rate of 0.9 LPM (liter per minute) was administered to the outer layer of the tumor by a perforated flask, such as shown in FIG. 10E., and as also illustrated in FIG. 22, for 10 minutes. Then, the skin was removed from the tumor, and the tumor was re-exposed to gNO via a perforated flask filled with gNO using the same parameters.

NO-2 is a male Balb/c mouse and was treated 3 times. On day 0 the tumor volume was 38.51 mm³. Day 0—gNO at 50,000 ppm was administered for 3.8 minutes at a flow rate of 0.9 using a cap. Day 5: gNO at 10,000 ppm was administered for 30 minutes using a cap. The shell was removed from the original tumor and gNO was delivered for 10 minutes using a cap, such as shown in FIG. 11C. Then, gNO was delivered intra-tumorally by a 23G needle such as shown in FIG. 11B at 0.3 LPM for another 10 minutes, using an intra-tumoral channel. The tip of the needle was directed at the outside area. Lastly, gNO was delivered for 10 minutes intra-tumorally by a 23G needle such as shown in FIG. 11B. Day 11: gNO at 10,000 ppm was delivered to the tumor for 5 minutes and at a flow rate of 0.3 LPM, using a 23G needle and an intra-tumoral channel.

The obtained data is presented in FIG. 9 and shows that the air-treated tumor is more than 30 times bigger than the gNO-treated tumors 12 days post treatment 1.

In an additional set of experiments, aluminum disposable gas cylinders were used. A flow meter was connected directly to the cylinder, to which a gas delivery tubing was connected. The tumor was inserted into a small flask trough a 1 cm hole and a plastic holder. A small hole at the bottom of the flask enabled to lower the pressure inside the flask. The gNO tubing was connected to the cap of the flask as shown in FIG. 10E. The flask was filled with gNO at 10,000 ppm in N₂ at a flow rate of 0.9 Liter Per Minute (LPM) for 20 minutes: 10 minutes without the removal of the skin from the tumor and additional 10 minutes after removal of the skin with a scalpel.

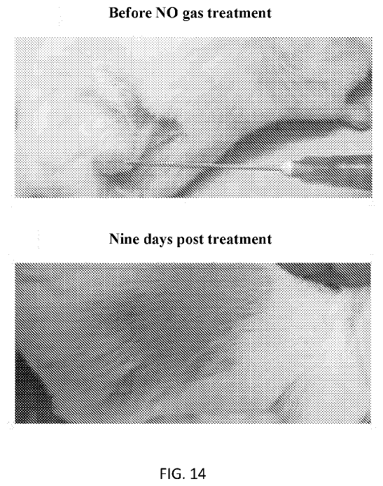

FIGS. 10A-D present photographs taken before treatment (FIG. 10A) and 1 minute (FIG. 10B), 9 days (FIG. 10C) and 14 days (FIG. 10D), post treatment. As can be seen, the tumor was destructed by the gNO treatment.

FIG. 10F presents the tumor volume following the treatment and shows 100% CT-26 tumor destruction 1-17 days post treatment with 10,000 ppm gNO.

In an additional set of experiments, stainless steel needles were used for intra-tumoral administration. Two types of gas cylinders were used—aluminum disposable and non-disposable cylinders.

For the non-disposable cylinders, a pressure regulator was connected directly to the cylinder, which was connected to the flow meter that was connected to the gNO tubing, to which a 23G hypodermic stainless steel was connected.

For the disposable cylinders, a flow meter was connected directly to the cylinder, to which a 23G hypodermic stainless-steel needle was connected through a gas delivery tubing, such as illustrated in FIG. 23. gNO at two doses was used: 10,000 or 50,000 ppm gNO in N₂, the flow rate was set to 0.1-0.3 Liter Per Minute (LPM) and the exposure time was 2-10 minutes in a pulsed manner. Additionally, gNO at 10,000 ppm-50,000 ppm was administered for 4 minutes to the outer layer of the tumor at 0.3-1 LPM by a cap. An air treated tumor bearing mouse served as the control group.

Treatment I (day 0) and II (day 8) were applied to all mice. Treatment III (day 14) was applied to mouse NO-5 only.

Treatment I: gNO at 50,000 ppm was administered, intra-tumoral by a 23G hypodermic needle, such as shown in FIG. 11B for 2-3 minutes at a flow rate of 0.2 LPM using a 3L cylinder.

Treatment II: gNO at 10,000 ppm was administered for 6 minutes intra-tumorally by a 23G needle, such as shown in FIG. 11B. Then, gNO at 10,000 ppm was administered for 4 minutes to the outer layer of the tumor by capping, such as shown in FIG. 11C. The flow rate was 0.3 LPM.

Treatment III: gNO at 50,000 ppm was administered by a cap such as shown in FIG. 11C to about 1 cm×0.5 cm cut at a flow rate of 0.9 LPM using a 3L cylinder for 10 minutes.

The obtained data is presented in FIG. 11A and shows CT-26 tumor growth inhibition in ⅔ mice in response to gNO treatment compared to air treatment. These mice were treated with nitric oxide at 50,000 ppm administered for 2-3 minutes at a flow rate of 0.2 LPM using a 3L cylinder. Nine days later nitric oxide at 10,000 ppm was administered for 6 minutes intra-tumorally by a 23G needle. Then, nitric oxide at 10,000 ppm was administered for 4 minutes to the outer layer of the tumor.

### Example 12

The effect of continuous gaseous nitric oxide (gNO) at 50,000 ppm on a solid CT-26 tumor ex-vivo was tested.

A 65 mm³ CT-26 tumor was excised and stored in PBS at 25° C. for 1 hour. One half of the tumor was exposed to 50,000 ppm gNO in an acrylic glass box at a volume of about 1.7 L for 6 minutes, 0.9 LPM. The remaining portion of the tumor was exposed to air.

A non-disposable NO cylinder was used for delivering gNO. A pressure regulator was connected directly to the cylinder. A flow meter was connected to the regulator. The gas delivery line was connected to the flow meter. The NO gas tubing was inserted to the box through a 1 cm hole. An additional 0.2 cm hole permitted the reduction of the pressure inside the box.

FIGS. 12A-B present the obtained data, and demonstrate the effect of continuous exposure of a CT-26 solid tumor to 50,000 ppm gNO or air, ex-vivo. FIG. 12A presents the outer layer of the nitric oxide exposed tissue. FIG. 12B presents the inner part of a nitric oxide exposed tissue.

### Example 13

BALB/c male mice were obtained from Almog Diagnostic Ltd. (Almog). Almog purchased the mice from Envigo, Israel. The mice were 8-10 weeks of age. Animal care and experimentation were performed in accordance with the Bar-Ilan University guidelines. Institutional Animal Care and Use Committee (IACUC) approval number #67-09-2019.

Mice were inoculated with CT26 cancer cells. CT26 is a colon carcinoma cell line induced in a BALB/c mouse by a chemical carcinogenesis. Cells were cultured in Roswell Park Memorial Institute (RPMI) 1640 supplemented with 100 U/ml penicillin, 100 μg/ml streptomycin, 2 mM L-glutamine; 1 mM sodium pyruvate; 1% non-essential amino acid solution; 10% fetal calf serum. Cultures were maintained at 37° C. in a humidified incubator with a 5% C02 atmosphere.

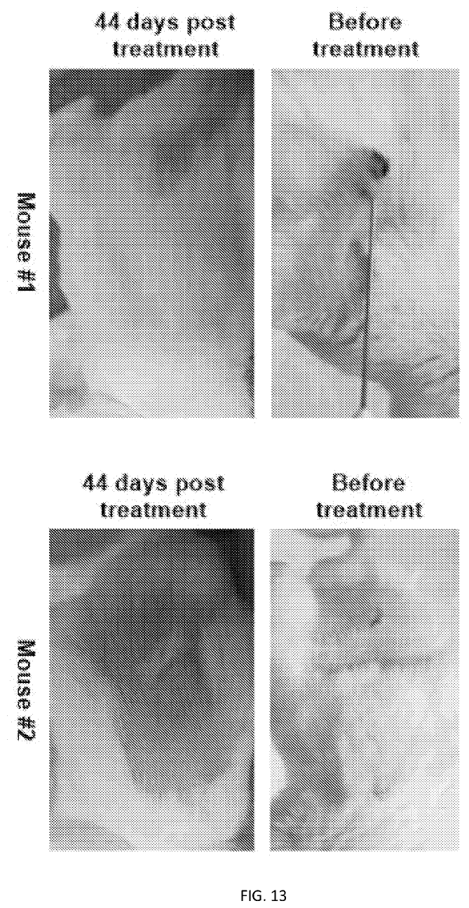

Mice were inoculated subcutaneously with 5×10⁵ CT-26 cells suspended in 100 L Hank's Balanced Salt Solution (HBSS) into the right flank, as shown in FIG. 13 (see, for example, upper right photograph). Local tumor growth was determined by measuring two mutually orthogonal tumor diameters with a caliper. The volume of tumor was calculated using the formula: Vol=D1×D2×D2/2, where D1, D2 are two mutually orthogonal tumor diameters.

CT26 tumor bearing mice were treated with 25,000 ppm gNO.

The gas was delivered into the tumor by a 23G hypodermic needle for 15 minutes, at a flow rate of 0.25 LPM and outlet pressure of about 3.5 Bar using standard pressure regulators and flow meters. A week later, all mice were re-treated using the same protocol.

FIG. 13 presents photographs of 2 mice of the gNO treated group (out 4 mice in the group) which were cured (50%). The photographs show the cured flank of these mice 44 days post the first treatment.

### Example 14

The tumor of a CT26 tumor bearing mouse was exposed to 200,000 ppm gNO (200,000 ppm gNO in 800,000 ppm nitrogen) for 2 seconds using a digital mass flow controller set to 0.01 LPM. In this setting, a valve is placed next to the pressure regulator, enabling manually stopping the flow. The valve was opened when the needle was in the tumor, resulting in an immediate swelling of about 1-2 cm³ of the tumor. The treatment was immediately stopped, and the mouse was transferred to its cage for recovery.

FIG. 14 presents photographs of the treated area before (upper photograph) and 9 days after (lower photograph) the gNO treatment, and show that that the tumor disappeared.

### Examples 15-17

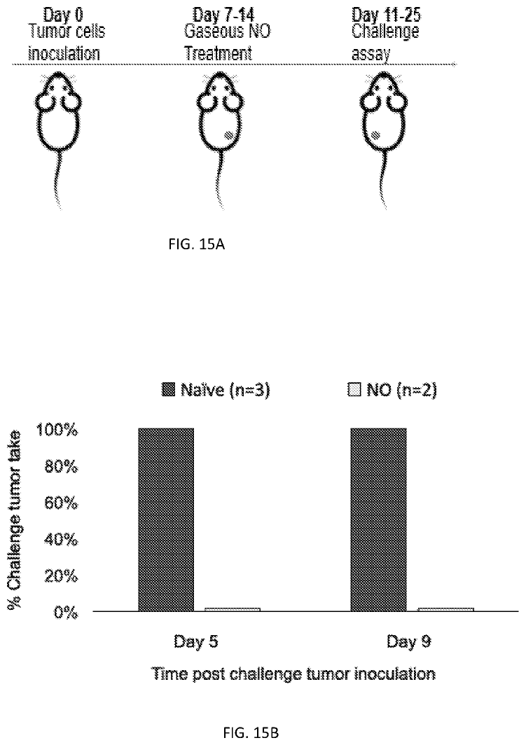

gNO local administration was tested in a Challenge assay, as depicted in FIG. 15A, in order to evaluate whether treating the primary tumor by gNO based ablation stimulates an anti-tumor immune response that results in rejection of a secondary tumor inoculation. A secondary, “challenge”, tumor cells inoculation also serves as a metastasis model.

LLC1 tumor-bearing mice were treated with 50,000 ppm NO administered intra-tumorally by a 23G hypodermic needle for 10 minutes. Up to 2 weeks post the first gas treatment all gas-treated mice were re-inoculated with 2.5×10⁵ LLC1 cells suspended in 100 μL HBSS into the left flank. Naïve mice inoculated with 5×10⁵ CT-26 cells served as an internal control for cells' quality.

The obtained data is presented in FIG. 15B, and show the % of mice that developed a secondary tumor, as determined via palpation of the tumor cell inoculation site.

As can be seen, all naïve mice developed a tumor already 5 days post challenge tumor inoculation, whereby none of the gas-treated mice developed a secondary tumor, also 9 days post challenge tumor inoculation.

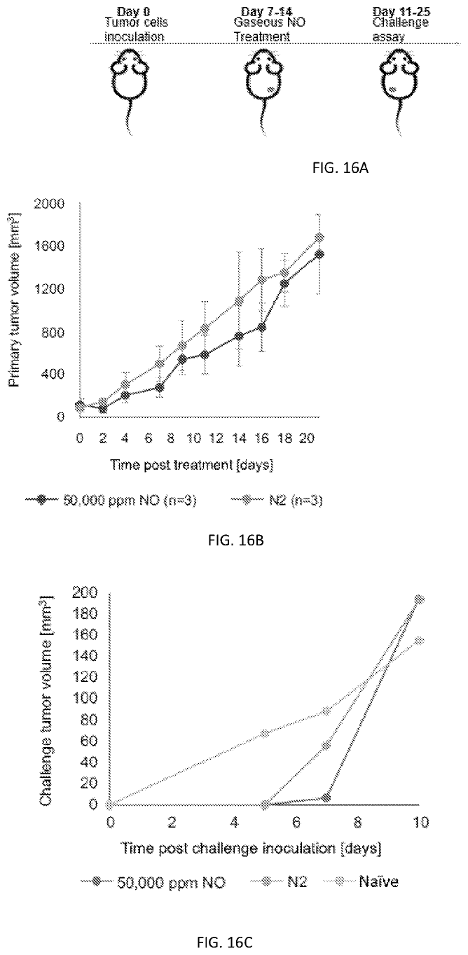

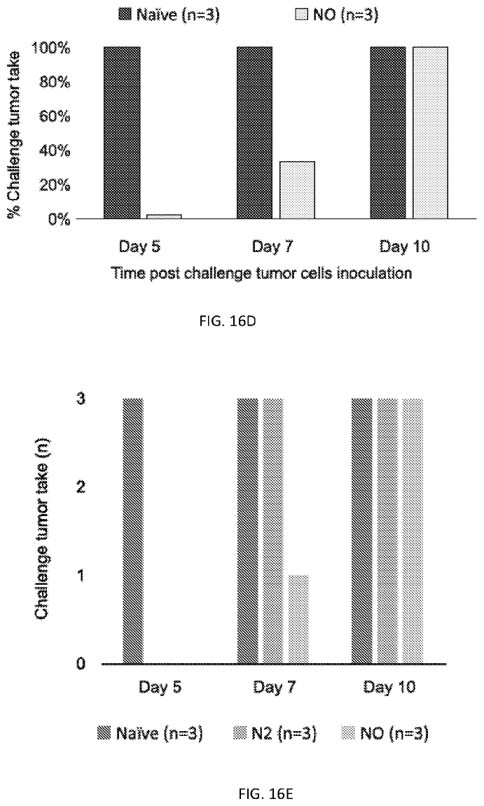

In another challenge assay, 4T1 tumor-bearing mice were treated with 50,000 ppm gNO administered intra-tumorally by a 23G hypodermic needle for 10 minutes, and the effect of a local treatment with gNO on the primary tumor as compared to nitrogen was tested, as depicted in FIG. 16A. Fourteen days post gNO treatment, mice were re-inoculated with 10×10^{5 4}T1 cells subcutaneously (s.c.) (challenge assay). The percentage of tumor take was monitored by caliper measurements 2-3 times a week for 10 days. Naïve mice, inoculated with 4T1 for the first time, served as the control group. Up to 2 weeks post the first gas treatment all gas treated mice were re-inoculated with 10×10⁵4T1 cells suspended in 100 μL HBSS into the left flank. In addition, naïve mice inoculated with 10×10′ 4T1 cells served as an internal control for cells' quality.

FIG. 16B presents the change in tumor volume following the first gNO local administration.

FIGS. 16C-16E present the average challenge tumor volume. As can be seen in FIG. 16C, all naïve mice developed a tumor 5 days post challenge tumor inoculation, while all the gas-treated mice (gNO—and nitrogen-treated mice) have not developed a secondary tumor 5 days post challenge tumor inoculation. As can be seen in FIGS. 16D and 16E, a delay in the challenge tumor take was achieved via gNO treatment, compared to non-treated nice and nitrogen-treated mice.

In another challenge assay, the tumors of CT26 tumor-bearing mice were treated with 25,000-200,000 ppm gNO delivered into the tumors using 23G hypodermic needles, as depicted in FIG. 17A.

CT26 tumor-bearing mice treated with 25,000 ppm for 30 minutes, were re-inoculated with CT26 tumor cells 12 days after treatment (n=1) and 14 days after treatment (n=1). The percentage of visible challenge tumor take was assessed 2-3 times a week for more than 50 days.

An additional group of CT26 tumor-bearing mice were treated with 50,000 ppm for 10 minutes, and were re-inoculated with CT26 tumor cells 7 days (n=1) or 9 days (n=1) after treatment. The percentage of challenge tumor take was assessed 2-3 times a week for 12 days.

An additional group of CT26 tumor-bearing mice were treated with 200,000 ppm for 2-35 seconds, were re-inoculated with CT26 tumor cells 14 days (n=2) post treatment. The percentage of visible challenge tumor take was assessed 2-3 times a week for more than 50 days.

Naïve mice, inoculated with CT26 for the first time, served as the control group.

The obtained results at the end of the follow-up period are presented in FIG. 17B and show that all NO-treated mice rejected a secondary CT26 tumor cell inoculation as compared to 100% tumor take in the naïve mice control group.

### Examples 18 and 19

Balb/c mouse was inoculated with 500,000 CT26 cells to the right flank. CT26 tumor was treated with 25,000 ppm NO for 15 minutes at a flow rate of 0.25 LPM. Seven days post gas treatment, this mouse was treated once again using the same protocol. Twelve days post the first NO treatment, this mouse was re-inoculated with 500,000 CT26 cells to the left flank. A third inoculation of cancer cells was performed 30 days post the second cell inoculation, using mouse breast cancer cells, was performed as well and the growth of a third inoculation of breast cancer cells, 4T1 cells. Forty-four days post challenge assay start (second inoculation), this mouse was sacrificed, and his spleen was harvested and preserved in RPMI-based media at 4° c. until processing of it. The spleen was processed into a sterile 35 mm culture dish containing 5 ml of RPMI-based media. Any extra connective tissues or fat was trimmed from the spleen. The flat end of a plunger was used to mince the spleen by crushing the spleen to release the splenocytes. A 70 μm strainer was primed by passing 1 ml of PBS through it and placed on a sterile 50 mL conical tube. The pellet was collected and transferred through the strainer via a 5 mL serological pipette. The strainer was washed with 3 mL of RPMI-based media. Tube will be centrifuged at 300×g for 10 minutes. The supernatant was discarded and resuspended in Ammonium-Chloride-Potassium (ACK) based solution to lyse red blood cells. After incubation, RPMI-based media was added, and cells will be centrifuged twice at 300× g for 10 minutes.

The splenocytes extracted from the spleen of CT26 immunized mouse were mixed with CT26 cells at a ratio of 1 CT26 cells:2-10 splenocytes cells and inoculated to naïve mice.

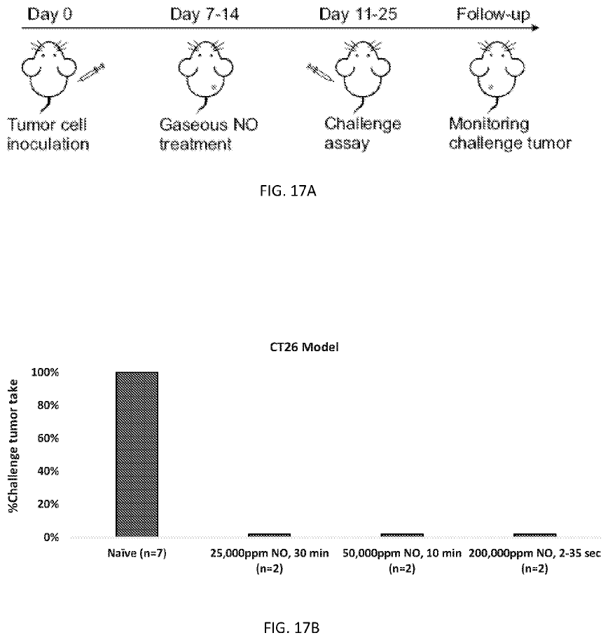

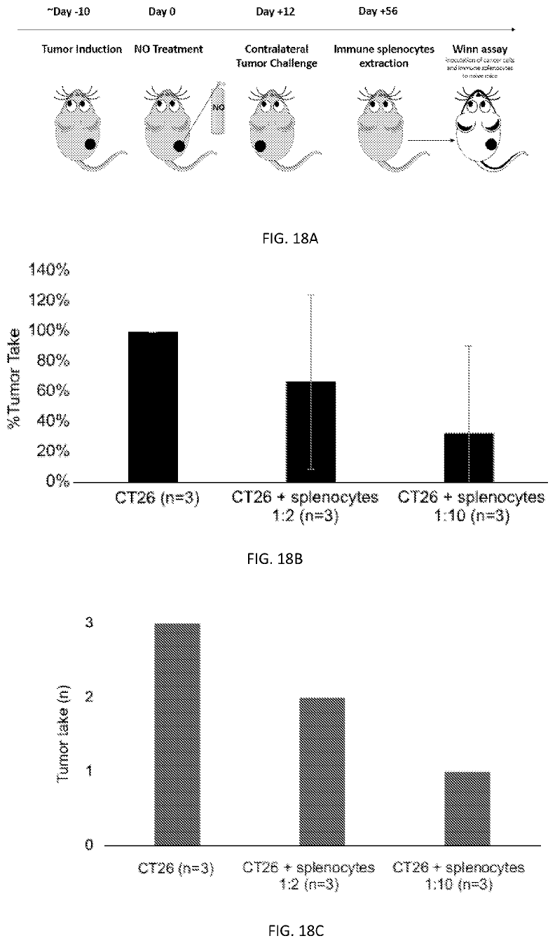

The assay scheme is presented in FIG. 18A.

A third inoculation of 4T1 cancer cells, using mouse breast cancer cells, was performed as well and the growth of a third inoculation of breast cancer cells was inhibited.

The obtained data is presented in FIG. 18B and FIG. 18C. As can be seen in FIG. 18B, the growth of wild type CT26 cells that were inoculated to naïve mice together with splenocytes extracted from CT26 immunized mice is inhibited, compared to wild type CT26 cells that were inoculated to naïve mice alone. As can be seen in FIG. 18C, the growth of CT26 cells that were mixed with splenocytes at a ratio of 1:2 was inhibited in 33% (1 out of 3 mice) of inoculated mice 30 days post inoculation, and the growth of CT26 cells that were mixed with splenocytes at a ratio of 1:10 was inhibited in 67% (2 out of 3 mice) of inoculated mice 30 days post inoculation.

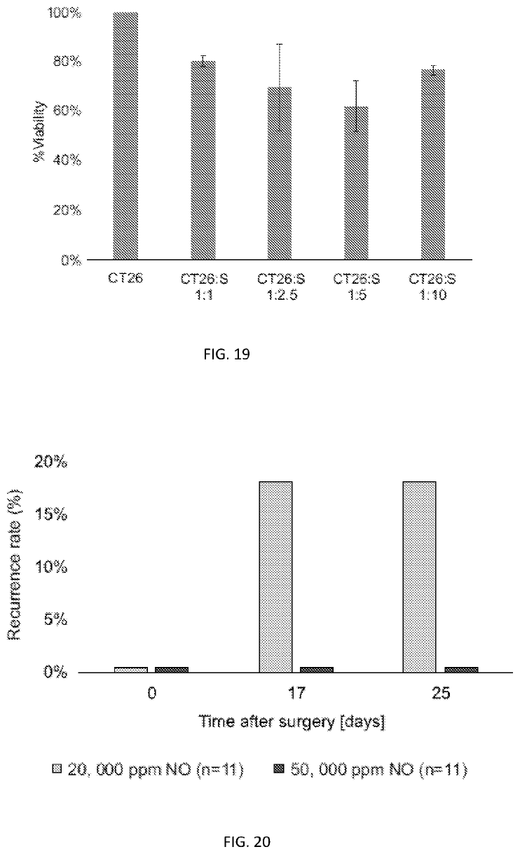

The potency of the extracted splenocytes to eliminate wild-type CT26 cells in-vitro was also tested by plating 10,000 CT26 cells per well in a 96-well plate and adding splenocytes at a ratio of 1-10 splenocytes to a single CT26 cell. CT26 cells viability was tested after 46 hours incubation by XTT. The obtained data is presented in FIG. 19 and show that about 60-70% of CT26 cells remain viable upon incubation with splenocytes at a ratio greater than 5. A dose-response effect is seen only in 1:1 to 1:5 ratios.

### Example 20

The following in vivo experimental procedures were carried out in accordance with the protocol approved by the Ethics Committee on the Use and Care of Animals. Institutional Animal Care and Use Committee (IACUC): IL-20-3-149.

CT26 tumor-bearing mice were treated with 20,000 ppm and 50,000 ppm gNO intra-tumorally for 5 minutes. Up to fourteen days post gNO treatment, all tumors were excised, and the recurrence rate was monitored for 89 days post-excision. Tumor recurrence was detected 17 days post treatment.

The obtained data is presented in FIG. 20 and show that about 18% of the 20,000 ppm gNO-treated tumor recurred 17 days post-surgery (excision), while 0% recurrence was observed in the 50,000 ppm gNO-treated group, 17 days post surgery (FIG. 20).

### Example 21

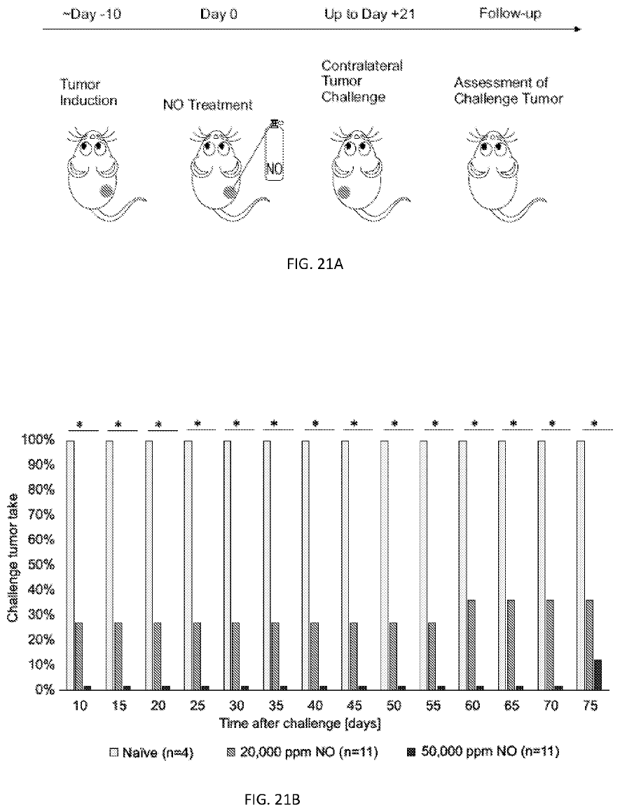

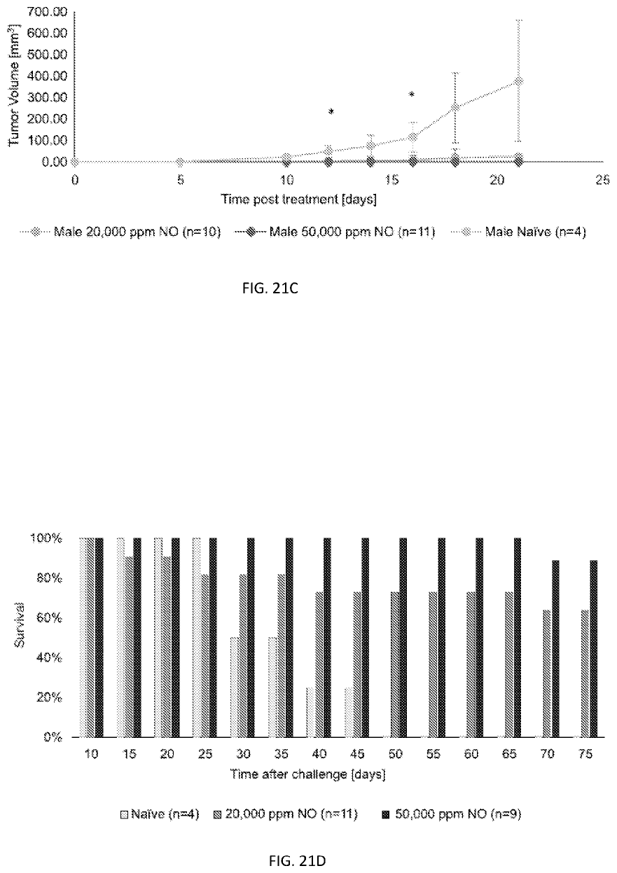

CT26 tumor-bearing male mice were treated with 20,000 ppm and 50,000 ppm gNO intra-tumorally for 5 minutes. Fourteen days post gNO treatment, all tumors were excised. A week post surgery, all mice were re-challenged via a second inoculation of 5×10⁵ CT26 cells, and tumor take and survival were monitored during about 75 days, as depicted in FIG. 21A. FIGS. 21B-D present the data obtained for the percentage of challenge tumor take was monitored (FIG. 21B), the challenge tumor volume (FIG. 21C) and survival (FIG. 21D), and clearly show no tumor development in the gNO-treated, and 100% survival in mice treated with 50,000 ppm gNO, compared to nearly no survival in non-treated mice.

### Example 22

The effect of high-pressure gas treatment on stimulation of anti-tumor immune response was tested in a challenge assay as depicted in FIG. 37A.

Balb/c mice were inoculated subcutaneously (s.c) with the mouse colon cancer cell line, CT26. CT26 tumor-bearing mice were treated 25,000 ppm gNO, air or nitrogen supplied by Gordon Gas and Chemicals in 3.5-liter cylinders. The outlet pressure was set to 3.5-5.17 bars. Gas was delivered to the tumors via a 23G needle and at a flow rate of 0.25 liter per minute (LPM) for two 15-minute cycles. At days 12-14 post gas treatment, all mice were re-inoculated s.c with CT26 tumor cells to the contralateral flank of all gas-treated mice. Naïve mice inoculated with CT26 cells for the first time served as a control to ensure that these cells were not defected on challenge day.

This challenge assay serves as model for metastasis development (secondary tumor). The obtained data is presented in FIG. 37B and show that while all naïve mice developed a tumor and were sacrificed when their tumor volume exceeded 2 cm³, three weeks post cells inoculation, all the gas-treated mice have not developed a secondary tumor. 50% of the gNO treated mice were tumor-free, with no primary and challenge tumors; 20% of the air or nitrogen-treated mice did not develop both visible primary and challenge tumors.

### Example 23

The gNO treatment as described herein is utilized for preparing a personalized ex-vivo nitric oxide-based cancer vaccine. An autologous tumor or metastasis specimen is exposed to nitric oxide outside of the patient's body. The resulting nitric oxide-treated pool of cancer antigens is administered back to the patient for the purpose of engaging the immune response to react against systemic malignant cells.

A patient can be treated simultaneously with: (I) In situ nitric oxide cancer vaccination as described herein in any of the respective embodiments and is demonstrated in Examples 15-21 herein; (II) Ex vivo nitric oxide-based cancer vaccination as described herein in any of the respective embodiments and detailed in this example; and (III) Immune-stimulating agent or any other additional agent or adjuvant as described herein in any of the respective embodiments. In general, in this cancer vaccination technique, the physician removes a tumor tissue sample from the patient. This sample is exposed to gaseous nitric oxide, for example, at from about 100 ppm to about 1,000,000 ppm, of from about 1,000 ppm to 1,000,000 ppm, or from 10,000 to 200,000 or from 20,000 to 200,000, or from 50,000 to 200,000 ppm, for a tome period of from 1 second to 10 hours, or from 1 minute to 2 hours, or from 1 minute to 60 minutes or from 10 minutes to 60 minutes, including any intermediate values and subranges therebetween.

The resulting nitric oxide-treated biomaterial is further purified to protein/peptide solution/non-viable cancer cells solution, or introduced to dendritic cells (DCs) to have loaded cancer antigen presenting cells.

The following describes an exemplary, non-limiting, procedure for preparing an ex vivo nitric oxide-based preparation of an individual cancer vaccine (personalized vaccine).

Leukocytes and DCs Purification from Blood Samples:

Whole blood is collected from the patient by venipuncture in Ethylenediaminetetraacetic acid (EDTA)-treated collection tubes. Blood cells are then separated out in a process that includes centrifugation of all blood cells, followed by red blood cell lysis, while following methodologies known in the art. In an exemplary, non-limiting procedure, blood sample is centrifuged, for example, at about 250-350× g for, for example, about 5-10 minutes. Then, red blood cell lysis is performed via an appropriate buffer, such as, but not limited to, Ammonium-Chloride-Potassium (ACK). About 1-20 ml of the buffer is added to 1-10 ml of blood cells at room temperature for, for example, about 1-10 minutes. The blood sample is then centrifuged, for example, at about 250-350× g for, for example, about 5-10 minutes. Supernatant is discarded, and cells are re-suspended in 1-10 mL cold buffer such as, for example, cold phosphate buffered saline. Blood cells are then washed once again by centrifugation, for example, at about 250-350× g for, for example, about 5-10 minutes. Supernatant is discarded, and cells are re-suspended in 1-10 mL cold buffer such as cold phosphate buffered saline. Dendritic cells (DCs) are optionally further purified using bead-conjugated antibodies, recognizing DCs markers, such as CD11c. Purified DCs are optionally proliferated using appropriate cell culture media and/or cryopreserved.

### Cancerous Tissue Sample Processing:

(A) One or more samples of tumor tissue(s) is/are collected from the patient and further processed in a nitric oxide-based procedure. A non-limiting exemplary procedure includes one or both of A and B as described below, followed by C and optionally D and/or E, as described below. Single-cell suspension preparation: The tissue sample is processed to a cell suspension using the appropriate enzyme mixture, typically, but not limited to, collagenase and DNase enzymes. The tissue is then processed automatically using a cell dissociation device.
(B) Peptide suspension preparation: The tissue sample is processed to a peptide suspension via homogenization, enzymatic digestion, and sonication of the sample.
(C) Sample exposure to gaseous nitric oxide: Cells or tissue samples are plated onto tissue culture plates or dishes and placed inside a nitric oxide compatible chamber. Nitric oxide is supplied into the chambers via, for example, gas cylinders or generators.
   (D) Purification: Bead-conjugated proteins of antibodies recognizing cell proliferation and viability markers are used to sort out active cancer cells. Alternatively, cancer cells are sorted out via appropriate protein and peptide filters. The output inactivated material is used for administration back to the patient.
(E) Cancerous material viability assessment: The viability of cells/tissues is assessed via standard cell viability assays, such as fluorescently labeling of death markers and measuring it via a flow cytometer, or cell proliferation-based assays. Inactivated material includes dead or non-proliferating cancer cells.
(F) Presentation of nitric oxide-treated cancer antigens to DCs from the patient: Obtained cancer antigens are introduced to DCs. For example, antigens obtained from previous steps are co-incubated with DCs from the patient, taken up and presented by them. The output antigen presenting DCs is used for administration back to the patient.
(G) Administration of the autologous cancerous nitric oxide processed material to the patient: The output material for administration back to the patient is either a solution, suspension, or implantation of a nitric-oxide treated tissue. The vaccination site is any part of the patient's body, such as, for example, the arm. The material is injected or implanted via any method, such as, but not limited to, intramuscularly, intratumorally, intraperitoneal, subcutaneously, intravenously. An additional agent can be injected before, simultaneously, or after cancerous material administration. This agent may be a cancer drug, an immune stimulating agent such as an adjuvant or an inhibitor of immune suppressor cells.

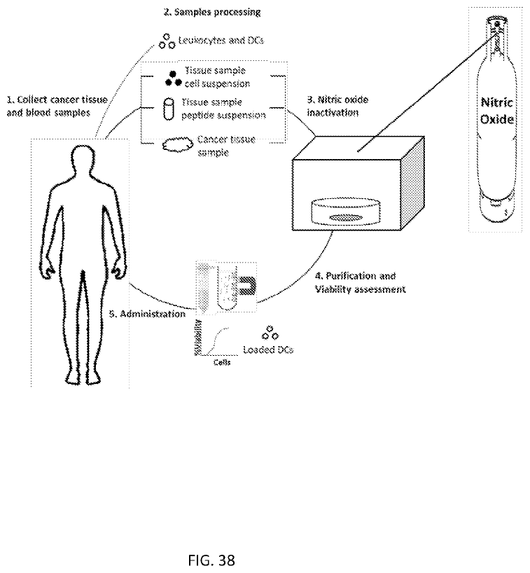

An exemplary, non-limiting preparation of a personalized gNO-based cancer vaccine is presented in FIG. 38, which shows a five-step process as follows:
1. Cancer tissue and blood samples collection
2. Sample processing: single-cell or peptide suspension preparation or slicing of the tumor tissue sample.
3. Nitric oxide inactivation of cancer tissue/cell/peptide samples: exposure of the processed tissue to gaseous nitric oxide (e.g., as described herein), supplied from a gas cylinder or generator.
4. Sample purification and viability assessment: purification of dead/non-proliferating cells followed by viability assessment and/or loading of DCs with cancer antigens.
5. Administration of the resulting nitric oxide-based vaccine to the patient

The gNO-based vaccine is tested in an animal model, for example, a mice model.

Mice at the age of 10-12 weeks are inoculated with cancer cells (˜10⁵-10⁶ cells per mouse) subcutaneously.

When the tumor volume is about 50 mm³−500 m³, the entire tumor or a sample is removed.

On tumor excision day, blood sample of about 100 μl-500 μl is collected.

The blood sample is centrifuged, for example, at 300× g for 10 minutes at room temperature; The pellet is re-suspended in 0.1-1 ml buffer, for example, ACK buffer; Blood cells are incubated with the buffer for 1-10 minutes; 1-10 ml of cold buffer (e.g., PBS) is added and cells are centrifuged, for example, at 300× g for 10 minutes at 4° C. Washing is repeated twice.

Bead-conjugated anti-CD11c antibodies are added and DCs are purified from the blood; DCs are grown in an appropriate cell culture media until use.

Alternatively, or in addition, cancer tissue samples are treated as follows: Sample is chopped, for example, by using a scalpel; Tissue is thereafter dissociated using appropriate suitable enzyme mixture and cell-dissociator, and is homogenized, enzymatically digested and sonicated to a peptide solution.

Alternatively or in addition, tumor samples are plated in tissue culture plates or dishes, placed inside a nitric oxide compatible chamber and exposed to gaseous nitric oxide as described herein. Control samples are exposed to nitrogen gas or remain untreated. Gas exposure is performed, for example, from 1 to 60 minutes, at a flow rate of, for example, 1-10 LPM.

Live/proliferating cells are thereafter sorted-out using bead-conjugated antibodies recognizing viability cell markers. Proteins and peptides are purified using appropriate filters.

DCs are incubated with the resulting nitric oxide-treated biomaterials Tumor-bearing mice are divided into groups. One group is injected intratumorally and/or intravenously with gNO-treated cells, non-viable gNO-treated cells, gNO-treated proteins, and/or DCs expressing gNO-treated antigens. Another group is injected intratumorally and/or intravenously with nitrogen-treated cells, non-viable nitrogen-treated cells, nitrogen-treated proteins, and/or DCs expressing nitrogen-treated antigens. A third group is injected intratumorally and/or intravenously with non-treated cells, non-viable non-treated cells, non-treated proteins, and/or DCs expressing non-treated antigens.

All mice are then re-inoculated with the same cancer cells.

Primary and secondary tumor volume and mice survival are monitored.

An exemplary, non-limiting, protocol for a clinical study of the designed gNO-based personalized vaccine is as follows.

A physician removes a 0.1-3 grams tumor tissue sample from the patient.

A physician/healthcare staff collects a 1-20 ml blood sample form the patient.

The blood sample is centrifuged, for example, at 300× g for 10 minutes at room temperature.

The pellet is re-suspended in a buffer (e.g., ACK buffer) an appropriate volume.

Blood cells are incubated with the buffer for 1-10 minutes. 1-10 ml of cold buffer (e.g., PBS) are added and cells are centrifuged, for example, at 300×g for 10 minutes at 4° C. this washing step is repeated twice.

Bead-conjugated anti-CD11c antibodies are added and DCs are purified/isolated from the blood.

The DCs are grown in appropriate suitable cell culture media until use.

The tissue samples are chopped using a scalpel, handled as described hereinabove and exposed to nitric oxide (e.g., 50,000 ppm for 10 minutes, at a flow rate of 1 LPM). Viability and proliferation of the cells is then evaluated as described hereinabove and live/proliferating cells are sorted out using bead-conjugated antibodies recognizing viability cell markers. Proteins and peptides are optionally also purified using appropriate filters.

The DCs are incubated with the obtained nitric oxide-treated biomaterials, and are administered to the patient by one or more of the following:

(I) DCs expressing nitric oxide-treated antigens are injected intravenously; (II) Dead nitric oxide-treated cancer cells and nitric oxide-treated protein/peptide solution are injected intramuscularly. (III) A mixture of (I) and (II) are injected into a specific tumor.

Total tumor burden in the patient is then assessed, for example via an imaging technique.

The tumor volume of the injected outgrowth is also assessed separately.

### Example 24

An exemplary system for local administration of gas, particularly, gNO, to a tumor (e.g., a tumor tissue) is described herein.

The system presented herein can effectively administer gaseous nitric oxide locally to treat tumors. Compared to conventional techniques, the system described herein is more targeted in treating tumors, particularly cancerous cells in vivo. The system described herein can effectively deliver gNO in treatment regimes, and can deliver gNO to target sites, with minimal damage, and preferably without damaging, healthy adjacent host cells, optionally and preferably while simultaneously identifying the target site and evaluating the effect of gNO local administration thereto.

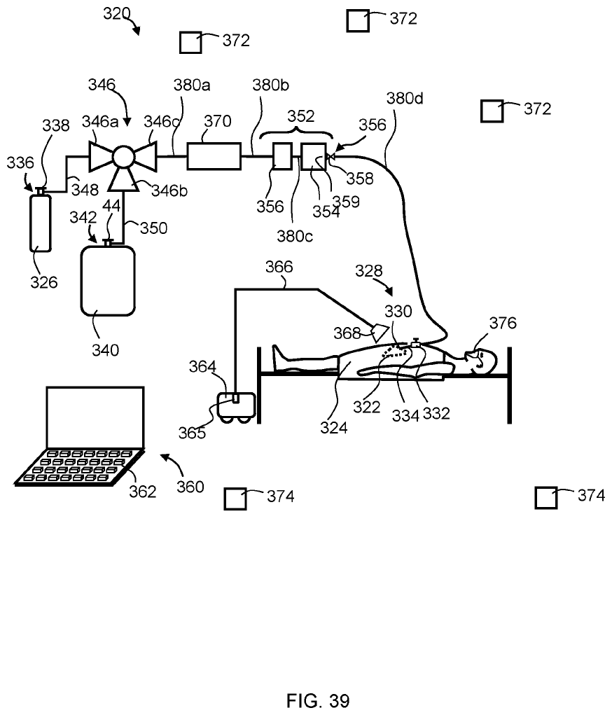

Referring now to the drawings, FIG. 39 illustrates a system 320 for delivery of gas to a tissue 322 of a subject 324, according to some embodiments of the present invention. The subject 324 is optionally and preferably a mammalian subject, more preferably a human subject. The tissue 322 is typically a tumor or a metastasis, or a physiological cavity containing same, or any other administration site as described herein, and the gas is delivered for the purpose of treating the tumor or a tumor metastasis. In some embodiments of the present invention the tumor is a malignant tumor.

System 320 can therefore be used to treat many types of cancers, as described herein in any of the respective embodiments.

The gas is optionally and preferably gNO, but other therapeutic gases are also contemplated.

System 320 comprises a first container 326 containing the gas, and an applicator 328 having a distal end 330 arranged to deliver the gas to the tissue 322. The gas is delivered to applicator 328 via a gas flow line shown at 380d. The applicator 328 can be of any type that has an outlet through which a flow of gas can exit. Typically, but not necessarily, applicator 328 is a transcutaneous device, e.g., a cannula 332 that ends in a needle 334 or a sprayer. The needle 334 can be any suitable needle for delivering the gas (e.g., gNO) including, but not limited to, a perforated spray needle, non-perforated and non-spray needle, umbrella needle, or other needles. The needle can optionally be nano size, micron size or macro size needles. Also contemplated are embodiments in which applicator 328 is configured for spraying, or otherwise exposing the tissue to the gas, in an open or closed container (e.g., a container sized to conform to the contours of the tumor), or to fill a space or a physiological cavity containing one or more tumors with the gas.

First container 326 typically comprises an outlet 336 and a valve 338 mounted thereon. First container 326 is optionally and preferably disposable. This is particularly advantageous when the gas is toxic, as in the case of gNO, so that the disposable container can be connected to system 320 immediately before treatment, and disposed immediately after treatment, thus reducing the time at which the toxic substance is in the treating room. In various exemplary embodiments of the invention the volume of container 326 is sufficient small (e.g., less than 100 cc, or less than 90 cc, or less than 80 cc, or less than 70 cc, or less than 60 cc or less than 50 cc) so that the amount of gas in container is not more than the typical gas dose to be delivered to the tissue. This is particularly advantageous when the gas is toxic, as in the case of gNO, because in the event of undesired leakage of the gas into the treating room, the total amount of gas that can be leaked is small, compared to the size of the room, thus reducing the risk of inhaling a hazardous concentration of the gas by the subject 324 or medical personnel.

For example, when the gas is gNO, the immediately dangerous to life or health (IDLH) concentration is 100 ppm, and so the amount of gNO in container 326 is preferably less than 1/10000 of a typical volume of a treating room, which is typically from about 40,000 liters to about 60,000. Thus, the volume of container 326 can be from about 10 cc to about 60 cc, and it can be filled with the gas at a volumetric concentration of from thousand ppm to several hundred-thousands ppm (e.g., 1,000-1,000,000 ppm), where “ppm” (parts per million) refers to the fraction (e.g., volumetric fraction) of the gas in a gas carrier. The gas carrier can be air, and preferably an inert gas such as nitrogen or argon, preferably nitrogen. In embodiments, the volume of first container 326 is from about 10cc to about 3.5L.

The gas pressure in container 326 is preferably low, e.g., less than 5 bar, e.g., from about 1 bar to about 5 bar. Alternatively, the gas pressure in container 326 can be higher (e.g., from about 5 bar to about 20 bar).

Thus, the first container 326 can comprise from about 1,000 ppm to 1,000,000 ppm of the gas, or any intermediate subrange therebetween, for example, from about 1,000 ppm to about 200,000 ppm, or from about 1,000 ppm to about 100,000 ppm, preferably from about 10,000 ppm to about 500,000 ppm, or from about 10,000 ppm to about 200,000 ppm, or from about 10,000 ppm to about 100,000 ppm, or from about 20,000 ppm to about 100,000 ppm, or from about 25,000 ppm to about 100,000 ppm, or from about 25,000 ppm to about 75,000 ppm, or from about 10,000 ppm to about 50,000 ppm, or from about 50,000 ppm to about 100,000 ppm, including any intermediate values and subranges between any of the foregoing, or is about 50,000 ppm.

System 320 can further comprise a second container 340 containing a purging gas, and having an outlet 342 and a valve 344 mounted thereon. Preferably, the purging gas is non-hazardous to the subject, more preferably an inert gas, such as, but not limited to, nitrogen or argon.

As the purging gas is non-hazardous, the volume of the second container 340 can be larger (e.g., 10 times or 100 times or 1000 times larger) than the volume of container 326. The gas pressure in container 340 is preferably sufficiently high to ensure efficient purging. Typically, the pressure in container 340 is at least 10 bar, or at least 20 bar, or at least 30 bar, or at least 40 bar, or at least 50 bar, e.g., from about 10 bar to about 200 bar.

Containers 326 and 340 are in fluid communication with a multi-port valve 346. In the schematic illustration of FIG. 1, which is not to be considered as limiting, valve 346 is embodied as a three-port valve having a first port, 346a, a second port 346b and a third port 346c. Valve 346 can be of any type, such as, but not limited to, a ball valve, a gate valve, a plunger valve, a butterfly valve or the like. Port 346a is typically in fluid communication with first container 336, port 346b is typically in fluid communication with second container 340, and port 346c is typically in fluid communication with applicator 328. In various exemplary embodiments of the invention the fluid communications between container 326 and port 346a, and between container 340 and port 346b are direct, namely that there are gas delivery lines 348 and 350 that respectively connect the containers 326 and 340 with the ports 346a and 346b, and there is no additional elements that interact with the respective gas along these lines. The connection of line 348 and optionally also of line 350 to the respective ports of valve 346 is preferably of the fast connection type.

Valve 346 is switchable between a first state at which port 346a fluidly connects to port 346c, and a second state at which port 346b fluidly connects to port 346c. During administration, valve 46 assumes the first state, and the gas flow from container 326 to the applicator 28 and into the tissue 322. Between administrations, more preferably before and after each administration, applicator 328 and optionally and preferably also container 326 are removed from system 320, and a purge step is executed by switching valve 346 to its second state, allowing the purging gas to enter the other components of system 320. The purging using the purging gas can contain one or several pressurizing and depressurizing cycles. The purging can, in some embodiments of the present invention, employ vacuum, as further detailed hereinbelow.

System 320 optionally and preferably also comprises a flow control system 352 for controlling the flow of gas exiting port 346c of valve 346. The gas is delivered to flow control system by a gas flow line shown 380b. Flow control system 352 is typically operated during treatment session and is switched off during the purge steps, but operating control system 352 during the purge steps is also contemplated. In embodiments, the flow control system 352 is configured, to deliver the gas for a time period of from about 1 second to about 60 minutes. In embodiments, flow control system 352 is configured to deliver a predetermined amount (volume and/or mass) of gas. In embodiments, flow control system 352 is configured to apply treatment in cycles. For example, flow control system 352 can pause the delivery after a predetermined amount of time and/or after a predetermined amount (volume and/or mass) of the gas has been delivered, and then, after a predetermined time interval, resume the delivery.

In some embodiments of the present invention flow control system 352 comprises an orifice 359 controlled by a valve 358, such as, but not limited to, an on/off valve, which in some embodiments of the present invention can be a solenoid valve.

In some embodiments of the present invention flow control system 352 comprises a flow controller 354 and a flow limiter 356. The gas flow from flow limiter 356 to flow controller 354 via a gas flow line shown at 380c.

Flow limiter 356 serves for limiting the flow rate (typically the volumetric flow rate) of the gas before entering flow controller 354. For example, flow limiter 356 can be an analogue flow controller, equipped with a knob (not shown) for setting an upper limit on the flow rate of the gas passing through limiter 356. The flow limiter can in some embodiments of the present invention include an orifice of a diameter selected to limit the maximum flow, thus serving as a flow restrictor. Typically, flow limiter 356 limit the gas flow rate to a value of from about 0.01 liters per minute (LPM) to about 0.15 LPM, more preferably from about 0.011 liters per minute (LPM) to about 0.11 LPM. Suitable devices for use as flow limiter 356 include the analogue flow controller VAF-G2-01L series, and the flow restrictor 6LV-4-VCR-6-DM series, both commercially available from Swagelok, USA.

Flow controller 354 is optionally and preferably a digital flow controller, more preferably a Proportional-Derivative (PD) controller or a Proportional-Integral-Derivative (PID) controller, configured for controlling the flow through controller 354 in closed loop. The closed loop control of controller 354 can be according to tuning coefficients. Specifically, controller 354 receives, as input, a value of the flow rate, and repeatedly measures the flow rate at its outlet 356. Controller 354 calculates the difference between the measured value of the flow rate and the input value of the flow rate, and then calculates a control signal using the calculated difference. The control signal is used by controller 354 to operate a valve 358 at its outlet 356. The control signal is optionally and preferably calculated as a weighted sum of the calculated difference, the time-derivative of the calculated difference, and optionally also the time-integral of the calculated difference. The weight of the calculated difference in the control signal is referred to as a proportional tuning coefficient, the weight of the time-derivative of the calculated difference in the control signal is referred to as the pseudo-derivative tuning coefficient and, the weight of the time-integral (when computed) of the calculated difference in the control signal is referred to as the integral tuning coefficient.

In some embodiments of the present invention the value of the proportional tuning coefficient is higher for lower input flow rates than for higher input flow rates, and in some embodiments of the present invention the value of the differential tuning coefficient is lower for lower input flow rates than for higher low input flow rates. Representative preferred ranges for the proportional (P) and pseudo-derivative (D) tuning coefficients, for several input flow rates are provided in Table A, below.

TABLE A

| Input flow rate [LPM] | P | D [minute⁻¹] |
|---|---|---|
| 0.05 | 2000-3000 | 200-250 |
| 0.25 | 500-1000 | 300-400 |
| 0.5 | 300-500 | 400-600 |
| 0.75 | 250-350 | 500-700 |

In some embodiments of the present invention system 320 comprises a computerized controller 360 having a circuit configured to automatically control flow control system 352. For clarity of presentation, control lines from and to computerized controller 360 are not illustrated. In some embodiments of the present invention computerized controller 360 is at the treatment room (the same room with the applicator 328). More preferably computerized controller 360 is outside the treatment room (controller 360 and applicator are at different rooms). The advantage of this embodiment, is that it reduces the risk of exposure to the gas by the medical practitioner accessing and/or operating computerized controller 360.

Computerized controller 360 controls flow control system 352 according to a predetermined gas flow rate, and/or a predetermined total amount of the gas flowing through system 352, and/or a predetermined total amount of time in which the gas flows through system 352. Computerized controller 360 can comprise a dedicated circuitry and/or a general purpose computer, configured for receiving data and executing the operations described below. Computerized controller 360 can also include a user interface 362 for receiving input from the operator. For example, controller 360 can receive via user interface 362 an input flow rate of the gas, and automatically select the tuning coefficients (e.g., according to Table A, above, or according to any other scenario).

Controller 360 can also receive via user interface 362 an input dose of the gas to be delivered to the subject 324 and transmits a control signal to flow control system 352 to ensure that the total amount of delivered gas does not exceeds the input dose. For example, controller 360 can receive from the digital flow controller 354 a monitoring signal pertaining to the amount of gas that exits outlet 356 and transmit a stop signal to system 352 once the amount of gas has reached the dose. In some optional embodiments of the invention computerized controller 60 also controls one or more of valves 346 and 338, to ensure that the amount of gas delivered does not exceeds the input dose.

In some embodiments of the present invention system 320 comprises a suctioning device 364 arranged to apply suction at a vicinity of the distal end 330 of applicator 328 to withdraw excess gas exiting distal end 330. The suctioning of the gas can be done in a pulsed or continuous manner. Device 364 preferably has an adjustable arm 366 having a suction inlet 368 at its end.

The length and/or orientation of arm 66 can be adjusted by the medical partitioned before beginning the treatment session such that the suction inlet 68 is at close proximity to the distal end 330 of applicator 368. In use, excess gas that does not enter tissue 322 is sucked into suction inlet 368 instead of being released to the environment. The withdrawn gas that enters the suction inlet 368 is optionally and preferably passed through a filter 365 selected to remove hazardous gas components such as gNO and NO₂. For example, filter 365 can be a Sodalime or alkaline activated carbon filter. Typically, a gas flow line (not shown) is mounted on or embedded in arm 366. Filter 365 can be installed in device 364, as illustrated in FIG. 39, or alternatively at a location along the gas flow line mounted on or embedded in arm 366. The gas withdrawn by device 364 can be evacuated to the medical center pipe (not shown).

In some embodiments of the present invention computerized controller 360 is configured also to control suctioning device 364. In these embodiments computerized controller 360 activates device 364 before the beginning of the treatment session, and deactivates it after the end of the treatment session.

It is to be understood that while FIG. 39 illustrates a single suction inlet 368, the present embodiments contemplate a plurality of suction inlets. For example, device 364 can include multiple suction inlets. Alternatively or additionally, system 320 can include a plurality of suctioning devices, each comprising one or more suction inlets.

In some embodiments of the present invention system 320 comprises an adjustable pressure regulator 370, in fluid communication with port 346c of valve 346. The gas flows from port 346c to regulator 370 via a gas flow line shown at 380a. Pressure regulator 370 is preferably configured for maintaining a pressure which is below a predetermined threshold when valve 346 assumes its first state (treatment session), and a pressure which is above the predetermined threshold when valve 346 assumes its second state (between treatment sessions). A typical pressure threshold employed by pressure regulator 370 is, without limitation from about 2 bars to about 5 bars. Pressure regulator 370 typically includes one or more pressure gauge devices (not shown) for providing indication regarding the gas pressure downstream and/or upstream the regulator 370.

System 320 is typically installed in a treatment room. Optionally, but not necessarily, the treatment room is sealed to the environment so as to ensure that the gas (e.g., gNO) does not leak out of the room. In various exemplary embodiments of the invention system 320 comprises an arrangement of sensors 732 distributed in the treatment room for sensing the gas. Sensors 372 are optionally and preferably configured for generating an alert signal when the level of the gas is above a respective predetermined threshold. In some embodiments of the present invention, the arraignment also include sensors 374 configured for sensing one or more reaction products of the gas. Sensors 374 are optionally and preferably configured for generating an alert signal when the level of the reaction product is above a respective predetermined threshold, which may be the same as the aforementioned threshold. For example, when the gas is gNO, sensors 374 can be configured for sensing NO₂, which is the reaction product of gNO with oxygen. The vertical locations of sensors 372 and 374 can be selected based on the specific density of the gas and reaction product to be sensed. For example, when sensors 372 sense gNO and sensors 374 sense NO₂, sensors 372 can be distributed at the upper part of the room, and when sensors 374 sense NO₂, sensors 374 can be distributed at the lower part of the room.

The predetermined threshold can be, for example, a value from about 25 ppm to about 100 ppm. Suitable sensors for sensing gNO are commercially available from, for example, Honeywell analytics, United Kingdom, and Watch Gas, The Netherlands.

In some embodiments of the present invention computerized controller 360 receives sensing signals from sensors 372 and/or 374 issues an alert signal when the level of the gas (e.g., gNO) and/or reaction product (e.g., NO₂) is above the respective predetermined threshold.

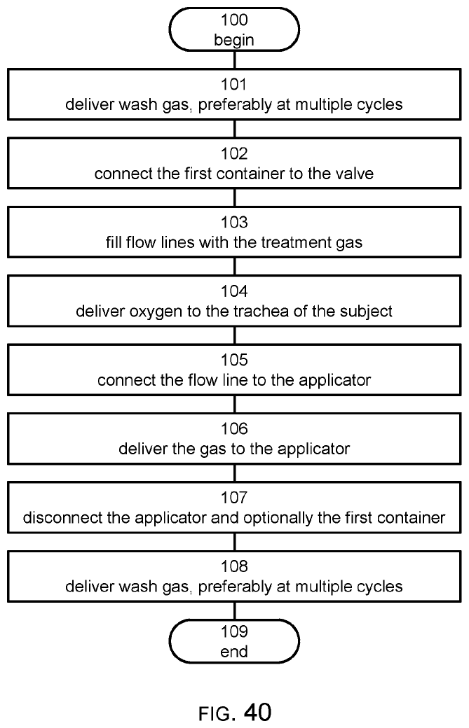

FIG. 40 is a flowchart diagram illustrating a method suitable for delivery of a gas to a tissue, according to some embodiments of the present invention. The method is executed using system 320.

The method begins at 100 and optionally and preferably continues to 101 at which the valve 346 is switched to second state to perform purging in which the various components and gas flow lines of the system (e.g., system 352, regulator 370, and gas flow lines 380a, 380b, 80c, 380d) are washed so as to purging gas remnants and other substances (e.g., oxygen) that can react with the gas. Preferably, the purging is for a predetermined time period, e.g., at least 1 minute for at least 3 times. In some embodiments of the present invention the purging includes multiple (e.g., 3 or more) pressurize and depressurize cycles, followed by continuous flow of the purging gas. The Inventors found that such a protocol speeds up the purge and ensures that gas remnants and other substances are more effectively purged out, even from dead ended gas pathways and corners. In some embodiments of the present invention the depressurizing parts of the cycles includes application of vacuum to the gas flow lines. This can be done, for example, by temporarily connecting one of the ports of valve 346 to a vacuum source (not shown). Alternatively, valve 346 can include an additional port (e.g., a fourth port, in which case valve 346 can be a four-port valve) to which the vacuum source is connected, and the depressurizing parts of the cycles can include switching the valve to a state in which the fourth port connects to the third port 346c.

In some embodiments of the present invention a gas leak detection solution is applied to connections through which gas is to be delivered for visual inspection of leakage by formation of bubbles.

The method optionally and preferably continues to 102 at which the first container 326 is connected to valve 346. If the first container 326 is already connected to valve 346, operation 102 can be skipped.

The method optionally and preferably continues to 102 at which the gas flow lines are filled with the gas (e.g., gNO). Preferably, this operation is executed before connecting gas flow line 380d to applicator 328, and while the end of the gas flow line 380d is positioned at a suction inlet (e.g., of device 364 or a vacuum source), so as to prevent release of the gas to the treating room.

In some embodiments of the present invention operation 103 is executed while monitoring the presence and/or concentration of the gas at the end of line 380d, for example, by a sensor, such as one of sensors 372, placed in proximity to the end of line 380d. In these embodiments, the delivery is terminated once the presence of the gas is detected and/or once the monitored concentration is above a predetermined threshold.

The method optionally and preferably continues to 104 at which oxygen or air or oxygen-enriched air is delivered to the subject, e.g., to subject's trachea, for example, by means of an oxygen mask 376 (see FIG. 39) placed on the subject's nose and/or mouth. In some embodiments of the present invention the medical practitioner preparing the subject for treatment and/or performing the treatment also wears a mask such as mask 376. The method continues to 105 at which the flow line 380d is connected to applicator 328. This is optionally and preferably executed after the applicator is already secured to a location near the tissue 322 and the needle 334 or sprayer already engages the tissue 322. At 106 valve 346 is again switched to its first state thereby delivering the gas to the tissue.

The method optionally and preferably proceeds to 107 at which the applicator 328 and optionally and preferably also the first container 326 are disconnected, and to 108 at which valve 346 is switched again to the second state to perform purging in which, the various components and gas flow lines of the system are washed so as to purging gas remnants and other substances (e.g., oxygen) that can react with the gas. Preferably, this operation is executed as described above with respect to operation 101. In some embodiments of the present invention at least one of operations 101 and 107 is executed while monitoring the presence and/or concentration of the gas at the end of line 380d, as further detailed hereinabove. In these embodiments, the delivery is terminated once the monitored concentration is zero or below a predetermined threshold.

The method ends at 109.

### Materials and Methods for Examples 25-26

Preparation of CT26 cancer cells for inoculation to mice—Cancer cell suspension, in HBSS (Biological Industries, Israel), at a concentration of 5.0×10⁶ cells/ml were freshly prepared on cell inoculation day. CT26 cells, at passage 8, were harvested using trypsin (Biological Industries, Israel) and counted using a hemocytometer. The numbers of viable and dead cells were recorded. The cells were then centrifuged at 1,200 round per minute (rpm) for 7 minutes, and the pellet was re-suspended in HBSS at 5.0×10⁶ cells (both viable and dead) per 1 ml HBSS solution.

Primary CT26 tumor induction—Cancer cell suspension at a concentration of 5.0×10⁶ cells/ml were inoculated to the right flank of animals at a volume of 100 μlper mouse (total 500,000 cells per mouse). Administration is performed as soon as possible following cell preparation and after manual shaking prior to withdrawal of cell suspension. Injections were performed using a 1 ml syringe and a 27G needle.

Tumor measurement—Local tumor growth was determined by measuring 3 mutually orthogonal tumor dimensions 2-3 times per week, according to the following formula: Tumor

$$Tumor volume = \frac{π}{6} \times [ Diameter 1 \times Diameter 2 \times Diameter 3 ] ;$$

where Diameter 1 is length, Diameter 2 is width, and Diameter 3 is height.

Tumor excision—Fourteen days post CT26 cell inoculation, CT26 tumors were excised. Prior to surgery, mice were anesthetized using an intra-peritoneal (i.p) injection of 100 mg/kg Ketamine and 10 mg/kg Xylazine. The tumors were disinfected using Ethanol 70%. Following, the tumor was lifted a bit via surgical tweezers and detached from the skin using surgical scissors. The area was further disinfected with Polydine. The incision was closed using medical clips. All surgical operations were followed by the administration of an analgesic supplied via drinking water of the mice: 0.5 gr of Optalgin (Dipyrone) in 250 ml of water for three days after surgery.

Exposure of CT26 tumors to NO—Following removal of the skin the tissue sample was placed immediately in a tissue culture plate. The plate was placed inside an NO exposure chamber, which is a ˜2L cube-shaped container, made of acrylic glass. Briefly, a pressure regulator was connected to the gas cylinder. To control the gas flow, a flow meter/controller was connected as well. The gas tubing was connected to the top of the chamber and the entire chamber was filled with gas. To limit gas leaks from the chamber, the top of it was further sealed with parafilm paper.

Three CT26 tumors were exposed to 200,000 ppm NO for 5 minutes, delivered at 0.25 liter/minute and a regulator outlet pressure of 2 bar served to allow gas flow.

Ex-vivo incubation of CT26 tumors—Following exposure to NO, tumor tissues were placed immediately in a tissue culture plate in sterile RPMI-based CT26 cell culture media (Biological Industries, Cat No. 30-2001) with 2% penicillin/streptomycin (i.e. 200 U/ml penicillin and 200 μg/ml streptomycin, Biological Industries, Cat No. 03-031-5B) and 10% fetal bovine serum for an overnight incubation at 37° c. and 5% CO₂. Following incubation and prior to transplantation, the tumor was cut into pieces about the size of a pencil eraser (5×5×5 mm).

Tumor tissue transplantation—Mice were anesthetized via an intra-peritoneal injection of 100 mg/kg Ketamine and 10 mg/kg Xylazine. The area from the mid-spine to the base of the tail was disinfected with ethanol 70%. A small, horizontal incision of 5 mm in length, 10 mm above the base of the tail in the left flank was made, using small surgical scissors. Directly over the flank the scissors were opened to introduce a pocket in the subcutaneous space. Following, few pieces of each tumor were inserted into the pocket created using forceps; and the incision site was closed with medical clips.

Challenge tumor inoculation—Twenty-one days post tumor tissue implanting, CT26 cells were prepared as described above. A cancer cells suspension at a concentration of 5.0×10⁶ cells/ml was inoculated to the left flank of animals at a volume of 100 μl per mouse (total 500,000 cells per mouse) as described above. Challenge tumor take was assessed viapalpation 2-3 times a week. The volume of challenge tumors was assessed as described above.

### Example 25

### Tumor Vaccination Using an Ex-Vivo Treated Tumor Sample

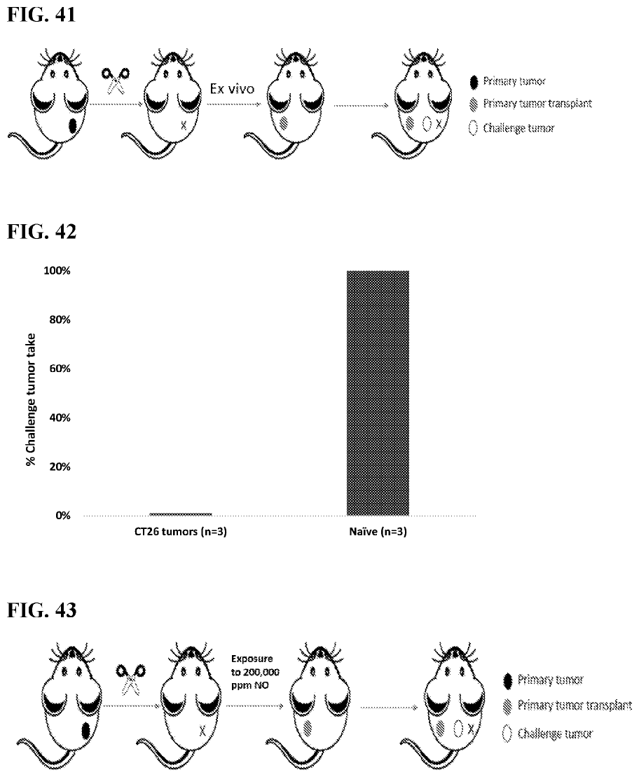

In the effort of developing a tumor vaccine, CT26 mouse colon tumors were induced in the right flank of mice and excised by surgery, treated ex-vivo and re-implanted back to the same mice in the left flank. The ex-vivo treatment included incubation of the tissues in RMPI cell culture media supplemented with 10% fetal bovine serum and 2% penicillin and streptomycin (two fold the standard concentration used for cancer cell growth) at 37° c. and 5% CO₂. To assess if an anti-CT26 immune response is stimulated following this procedure, a challenge assay was performed. Specifically, twenty-one days post re-implanting, mice were re-inoculated with non-treated CT26 cells injected to their lower back (FIG. 41).

Thirty days post the initiation of the challenge assay, the re-implanted mice were resistant to the challenge and have not developed a secondary tumor as compared to the control group of naïve mice that were inoculated with non-treated CT26 cells for the first time which all developed a tumor (FIG. 42).

### Example 26

Tumor Vaccination Using an Ex-Vivo Treated Tumor Sample In an effort of developing a tumor vaccine, CT26 mouse colon tumors are induced in the right flank of mice, allowed to grow and excised by surgery, treated ex-vivo and re-implanted back to the same mice in the left flank. The ex-vivo treatment includes 200,000 ppm gNO followed by incubation of the tissues in RMPI cell culture media supplemented with 10% fetal bovine serum and 2% penicillin and streptomycin (two fold the standard concentration used for cancer cell growth) at 37° c. and 5% CO₂. To assess if an anti-CT26 immune response is stimulated following this procedure, a challenge assay is performed. Specifically, twenty-one days post re-implanting, mice are re-inoculated with non-treated CT26 cells injected to their lower back (FIG. 43).

### Materials and Methods for Example 27

Preparation of CT26 cells prior to exposure to gas—4×10⁶ CT26 cells are plated 1 day prior to exposure to the gaseous nitric oxide (gNO) in 10 ml RPMI-based tissue culture media (Biological Industries, Cat No. 30-2001) supplemented with 1% Streptomycin-Penicillin (i.e. 100 U/ml penicillin and 100 μg/ml streptomycin, Biological Industries, Cat No. 03-031-5B) and 10% fetal bovine serum. All dishes are kept in an incubator adjusted to 37° c. and 5% CO₂ for an overnight incubation.

Exposure of CT26 cells to gNO—Immediately prior to exposure to gNO, cell culture media is removed using a disposable pipette. The dish is placed inside an NO exposure chamber, which is a ˜2L cube-shaped container, made of acrylic glass. Briefly, a pressure regulator is connected to the gas cylinder. To control the gas flow, a flow meter/controller is connected as well. The gas tubing is connected to the top of the chamber and the entire chamber is filled with gas. To limit gas leaks from the chamber, the top of it is further sealed with parafilm paper. 10,000 ppm, 50,000 ppm or 200,000 ppm (1%, 5%, or 20%, respectively) NO in nitrogen is delivered to the chamber for 2 minutes at a flow rate of 1 LPM. Immediately following exposure, the dishes are washed with 5 ml PBSX1. The tissue was placed inside the chamber.

Preparation of CT26 cancer cells for inoculation to mice—CT26 cells are harvested using trypsin (Biological Industries, Israel). The cells are then centrifuged at 1,200 round per minute (rpm) for 7 minutes. The numbers of viable and dead cells are recorded. The pellet is re-suspended in HBSS at 5.0×10⁶ cells (both viable and dead) per 1 ml HBSS solution. Following, the cell suspension is titrated with TRIS buffer to a pH of 6-8.

Vaccination—Cancer cell suspension at a concentration of 5.0×10⁶ cells/ml are inoculated to the right flank of animals at a volume of 100 μl per mouse (total 500,000 cells per mouse). Administration is performed as soon as possible following cell preparation and following manual shaking prior to withdrawal of cell suspension. Injections are performed using a 1 ml syringe and a 27G needle.

Tumor measurement—Local tumor growth is determined by measuring 3 mutually orthogonal tumor dimensions 2-3 times per week, according to the following formula:

$$Tumor volume = \frac{π}{6} \times [ Diameter 1 \times Diameter 2 \times Diameter 3 ] ;$$

where Diameter 1 is length, Diameter 2 is width, and Diameter 3 is height.

Tumor excision—Fifteen days post CT26 cell inoculation, CT26 tumors are excised. Prior to surgery, mice are anesthetized using an intra-peritoneal (i.p) injection of 100 mg/kg Ketamine and 10 mg/kg Xylazine. The tumors are disinfected using Ethanol 70%. Then, the tumor is lifted a bit via surgical tweezers and detached from the skin using surgical scissors. The area is further disinfected with Polydine. The incision is closed using medical clips. All surgical operations are followed by the administration of an analgesic supplied via drinking water of the mice: 0.5 gr of Optalgin (Dipyrone) in 250 ml of water for two days after surgery.

Challenge tumor inoculation—Twenty-one days post tumor tissue implanting, CT26 cells ae prepared as described above. Cancer cell suspension at a concentration of 5.0×10⁶ cells/ml are inoculated to the left flank of animals at a volume of 100 μl per mouse (total 500,000 cells per mouse) as described above. Challenge tumor take is assessed via palpation 2-3 times a week. The volume of challenge tumors is assessed as described above.

### Example 27

### Tumor Vaccination Using an Ex-Vivo Treated Tumor Cells

In the effort to develop a tumor vaccine, CT26 mouse colon cells are treated with gNO, suspended in medium or buffer and injected to the right lower back of mice twice, ten days apart of mice. As it was found that ex-vivo treatment of tumor cells with gNO results in an acidic pH of a cell suspension comprising the cells which may jeopardize activity and/or safety upon administration to a subject, the cell suspension is titrated with TRIS buffer to a pH of 6-8 prior to injection into mice. To further assess if an anti-CT26 immune response is stimulated following this procedure, a challenge assay is performed. Specifically, twenty-one days post re-implanting, mice are re-inoculated with non-treated CT26 cells injected to their left lower back.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of producing a tumor vaccine, the method comprising:
(a) ex-vivo exposing a tumor sample to gaseous nitric oxide (gNO);
(b) suspending said tumor sample in a medium or buffer subsequent to said exposing, so as obtain tumor cells in suspension; and
(c) titrating a pH of said suspension to 6-8,
thereby producing the tumor vaccine.

2. The method of claim 1, wherein said method does not comprise culturing said tumor sample or cells following step (a) step (b) and/or step (c).

3. A method of producing a tumor vaccine, the method comprising culturing a tumor sample in a medium comprising antibiotic at a concentration of at least 2 fold higher than the gold standard concentration for culturing primary cells of the same type as said tumor sample, thereby producing the tumor vaccine.

4. The method of claim 3, comprising ex-vivo exposing said tumor sample to gaseous nitric oxide (gNO).

5. The method of claim 4, wherein said exposing to said gNO is effected prior to said culturing.

6. The method of claim 4, wherein said exposing to said gNO is effected subsequent to said culturing.

7. A method of producing a tumor vaccine, the method comprising:
(a) ex-vivo exposing a tumor sample to gaseous nitric oxide (gNO); and
(b) culturing said tumor sample in a medium comprising antibiotic at a concentration of at least 2 fold higher than the gold standard concentration for culturing primary cells of the same type as said tumor sample,
thereby producing the tumor vaccine.

8. The method of any one of claims 1-7, comprising subjecting said tumor sample, said tumor cells or said tumor vaccine to a preservation protocol.

9. A method of producing a tumor vaccine, the method comprising:
(a) ex-vivo exposing a tumor sample to gaseous nitric oxide (gNO); and
(b) subjecting said tumor sample to a preservation protocol,
thereby producing the tumor vaccine.

10. The method of any one of claims 8-9, wherein said preservation protocol comprises freezing.

11. The method of any one of claims 7-10, wherein step (a) is effected prior to step (b).

12. The method of any one of claims 7-10, wherein step (a) is effected subsequent to step (b).

13. A method of preventing and/or treating a tumor in a subject in need thereof, the method comprising administering to the subject a therapeutically effective amount of a tumor vaccine, wherein said tumor vaccine is obtainable by ex-vivo exposing a tumor sample to gaseous nitric oxide (gNO), and wherein said administering is effected at least twice, thereby preventing and/or treating the tumor in the subject.

14. A method of inducing an immunological response to a tumor in a subject in need thereof, the method comprising administering to the subject a therapeutically effective amount of a tumor vaccine, wherein said tumor vaccine is obtainable by ex-vivo exposing a tumor sample to gaseous nitric oxide (gNO), and wherein said administering is effected at least twice, thereby inducing the immunological response to the tumor in the subject.

15. The method of any one of claims 13-14, comprising ex-vivo exposing said tumor sample to said gNO.

16. The method of any one of claims 3-8 and 10-12, wherein said antibiotic comprises penicillin-streptomycin.

17. The method of any one of claims 1-8, 10-12 and 16, wherein said medium comprises RPMI.

18. The method of any one of claims 3-8, 10-12 and 16-17, wherein said culturing is effected for at least 10 hours.

19. The method of any one of claims 3-8, 10-12 and 16-18, wherein said culturing is effected for less than 24 hours.

20. The method of any one of claims 1 and 4-19, wherein said exposing to gNO is at a dose of 1000-1,000,000 ppm.

21. The method of any one of claims 1-19, wherein said exposing to gNO is at a dose of 25,000-250,000 ppm.

22. The method of any one of claims 1-19, wherein said exposing to gNO is at a dose of about 200,000 ppm.

23. The method of any one of claims 1-22, wherein said exposing to gNO is for a time period of from 1 second to 100 minutes.

24. The method of any one of claims 1-22, wherein said exposing to gNO is for a time period of 1-15 minutes.

25. The method of any one of claims 1-22, wherein said exposing to gNO is for a time period of 4-6 minutes.

26. The method of any one of claims 1-22, wherein said exposing to gNO is for a time period of 1-3 minutes.

27. The method of any one of claims 1-25, wherein said exposing to gNO is at a flow volume of from 0.1 liter per minute (LPM) to 10 LPM.

28. The method of any one of claims 1-25, wherein said exposing to gNO is at a flow volume of from 0.1 liter per minute (LPM) to 1 LPM.

29. The method of any one of claims 1-28, wherein said exposing to gNO is at a flow volume of 0.5-1.5 liter per minute (LPM).

30. The method of any one of claims 1-28, wherein said exposing to gNO is at a flow volume of 0.2-0.3 liter per minute (LPM).

31. The method of any one of claims 1-30, wherein said exposing to gNO is effected in the absence of medium or buffer.

32. The method of any one of claims 3-12 and 15-31, comprising titrating pH of said tumor sample to pH 6-8 following said exposing to gNO.

33. The method of any one of claims 1-2 and 32, wherein said pH is 6.8-7.2.

34. The method of any one of claims 1-2 and claim 32-33, wherein said titrating said pH is effected by TRIS buffer, THAM buffer or NaOH base.

35. The method of any one of claims 3-12 and 15-34, further comprising processing said tumor sample to obtain a tissue fragment, single cells, clumps and/or an extracted biomaterial.

36. The method of claim 35, wherein said processing is effected subsequent to said exposing.

37. The method of claim 35, wherein said processing is effected subsequent to said culturing.

38. The method of claim 35, wherein said processing is effecting subsequent to said subjecting.

39. The method of any one of claims 3-38, wherein said tumor sample is a tissue sample.

40. The method of claim 39, wherein a volume of said tumor tissue sample is 1-1000 mm³.

41. The method of claim 39, wherein a volume of said tumor tissue sample is 1-200 mm³.

42. The method of any one of claims 1-38, wherein said tumor sample is a dissociated cells sample.

43. The method of any one of claims 1-38, wherein said tumor sample is a single tumor cells or clumps sample.

44. The method of any one of claims 35-38, wherein a volume of said fragment is 1-150 mm³.

45. The method of any one of claims 1-12 and 16-44, further comprising contacting antigen presenting immune cells with said tumor sample or said tissue fragment, single cells, clumps and/or extracted biomaterial processed therefrom to thereby obtain immune cells presenting an antigenic biomaterial of said tumor sample.

46. The method of any one of claims 1-45, comprising obtaining said tumor sample from a subject.

47. The method of any one of claims 1-46, wherein said tumor sample is obtained from multiple subjects.

48. The method of any one of claims 1-47, wherein said tumor sample comprises a primary tumor.

49. The method of any one of claims 1-47, wherein said tumor sample comprises a secondary tumor.

50. The method of any one of claims 1-47, wherein said tumor sample comprises a combination of a primary and a secondary tumor.

51. The method of any one of claims 1-12 and 16-50, further comprising determining viability and/or proliferation of said tumor vaccine.

52. The method of any one of claims 1-44 and 46-51, wherein tumor cells of said vaccine are non-proliferative and/or non-viable.

53. The method of any one of claims 1-12 and 16-51, further comprising administering a therapeutically effective amount of said tumor vaccine to a subject in need thereof.

54. A tumor vaccine obtainable by the method of any one of claims 1-12 and 16-52.

55. A method of preventing and/or treating a tumor in a subject in need thereof, the method comprising administering to the subject a therapeutically effective amount of the tumor vaccine of claim 54, thereby preventing and/or treating the tumor in the subject.

56. A method of inducing an immunological response to a tumor in a subject in need thereof, the method comprising administering to the subject a therapeutically effective amount of the tumor vaccine of claim 54, thereby inducing the immunological response to the tumor in the subject.

57. The method of any one of claims 13-53 and 55-56, wherein said therapeutically effective amount comprises 10-100 mg of said tumor sample or fragment processed therefrom.

58. The method of any one of claims 13-53 and 55-56, wherein said therapeutically effective amount comprises 0.1×10⁶-900×10⁶ cells.

59. The method of any one of claims 13-53 and 55-58, wherein said tumor sample and said tumor are of the same type.

60. The method of any one of claims 13-46, 48-53 and 55-58, wherein said tumor sample is autologous to said subject.

61. The method of any one of claims 13-53 and 55-58, wherein said tumor sample is allogeneic to said subject.

62. The method of any one of claims 45 and 55-61, wherein said antigen presenting cells are autologous to said subject.

63. The method of any one of claims 13-53 and 55-62, wherein said subject in need thereof has not been diagnosed with a tumor.

64. The method of any one of claims 13-53 and 55-62, wherein said subject in need thereof has been diagnosed with a primary tumor.

65. The method of any one of claims 13-53 and 55-62, wherein said subject in need thereof has been diagnosed with a secondary tumor.

66. The method of any one of claims 49-50 and 65, wherein said secondary tumor is a recurrent tumor.

67. The method of any one of claims 49-50 and 65, wherein said secondary tumor is a metastasizing tumor.

68. The method of any one of claims 53 and 55-67, wherein said administering is effected once.

69. The method of any one of claims 53 and 55-67, wherein said administering is effected at least twice.

70. The method of any one of claims 13-53 and 69, wherein a time interval between said at least two administrations is at least one week.

71. The method of any one of claims 13-53 and 69, wherein a time interval between said at least two administrations is at least six months.

72. The method of any one of claims 13-53 and 55-71, wherein said administering is into the arm.

73. The method of any one of claims 13-53 and 55-71, wherein said administering is intra-tumorally.

74. The method of any one of claims 13-53 and 55-73, further comprising administering to the subject an anti-cancer therapy.
